(19) United States
(12) Patent Application Publication
(10) Pub. No.: US 2005/0164323 A1
(43) Pub. Date: Jul. 28, 2005

(54) Methods of treating asthma

(75) Inventors: Divya Chaudhary, Andover, MA (US); Marion Kasaian, Charlestown, MA (US); Cara Williams, Methuen, MA (US); Suzana Marusic, Reading, MA (US); Robert M. Czerwinski, North Grafton, MA (US)

Correspondence Address: WILMER CUTLER PICKERING HALE AND DORR LLP, 60 STATE STREET, BOSTON, MA, 02109, US

(73) Assignee: Wyeth, Madison, NJ (US)

(21) Appl. No.: 11/022,327

(22) Filed: Dec. 22, 2004

Related U.S. Application Data
(60) Provisional application No. 60/532,525, filed on Dec. 24, 2003.
(60) Provisional application No. 60/589,415, filed on Jul. 20, 2004.

(51) Int. Cl. C12Q 1/68; A61K 38/00; C12N 5/08; C12Q 1/48

(52) U.S. Cl. 435/15; 514/2; 435/372

(57) ABSTRACT

Methods for agents useful for treating asthma are disclosed. The methods include screening for agents that inhibit the production of a PKC-θ protein, as well as for agents that inhibit the kinase activity of a PKC-θ protein, or a functional fragment thereof, wherein such agents are useful for treating asthma. The methods also include screening for agents that inhibit the production of a reporter gene product encoded by a nucleic acid sequence operably linked to a PKC-θ promoter. Also disclosed are methods of treating asthma that include administering an agent that inhibits the production of a functional PKC-θ protein or the kinase activity of a PKC-θ protein or a functional fragment thereof. An isolated mast cell lacking expression of endogenous PKC-θ is also disclosed.

### CROSS REFERENCE TO RELATED APPLICATIONS

This application claims the benefit of U.S. provisional application Ser. No. 60/532,525 filed Dec. 24, 2003, and of U.S. provisional application Ser. No. 60/589,415 filed Jul. 20, 2004, the entire contents of each of which is hereby incorporated by reference.

### BACKGROUND OF THE INVENTION

The present invention relates to the fields of biology and immunology. Specifically, the invention relates to asthma and methods for treating asthma.

Asthma is a chronic inflammatory disease of the airways characterized by recurrent episodes of reversible airway obstruction and airway hyperresponsiveness (AHR). The airways of patients with asthma are frequently sensitive and inflamed. When an asthma patient comes into contact with an allergen or something that irritates his airways, the airways constrict (i.e., the muscles around the walls of the airways tighten), making it difficult for the patient to breath. The lining of the airways become inflamed, leading to the production of phlegm and other clinical manifestations of allergy. Other clinical manifestations of asthma include shortness of breath, wheezing, coughing and chest tightness that can become life threatening or, in some instances, fatal.

While existing therapies focus on reducing the symptomatic bronchospasm and pulmonary inflammation, there is a growing awareness of the role of long term airway remodeling in accelerated lung deterioration in asthmatics. Airway remodeling refers to a number of pathological features including epithelial smooth muscle and myofibroblast hyperplasia and/or metaplasia, subepithelial fibrosis and matrix deposition. The processes collectively result in up to about 300% thickening of the airway in cases of fatal asthma. Despite the considerable progress that has been made in elucidating the pathophysiology of asthma, the prevalence, morbidity, and mortality of the disease has increased during the past two decades. The latest available data indicate that about 20 million people in the United States, and more than 150 million people worldwide, suffer from asthma. In the first part of this decade, in the United States alone, nearly 1.9 million emergency room visits, 454,000 hospitalizations and over 4,000 deaths were directly attributed to asthma on an annual basis.

It is generally accepted that allergic asthma is initiated by an inappropriate inflammatory reaction to airborne allergens. The lungs of asthmatics demonstrate an intense infiltration of lymphocytes, mast cells and especially eosinophils.

Although current research has revealed some of the complex cellular and molecular interactions that contribute to the inflammation observed in asthma, significant gaps of knowledge still exist.

As a result of research into the causes of asthma, a wide variety of drugs have become available to treat the symptoms of asthma. However, many of the drugs have various shortcomings that make them less than ideal for treatment of asthma. For example, many drugs, such as epinephrine and isoproterenol, only relieve the symptoms of asthma for a short period of time. Other treatments lose effectiveness after being used for a period of time. Additionally, some drugs, like corticosteroids, have severe side effects which limit their chronic use. There is a clear need, not only for an increased molecular understanding of asthma, but also for further beneficial asthma therapeutics. The present invention addresses these needs.

### SUMMARY OF THE INVENTION

The present invention is based, at least in part, on the inventors' discovery that protein kinase C theta (PKC-θ) plays a role in respiratory disease states, including asthma. Accordingly, the invention provides methods for identifying agents useful for treating asthma, methods for treating patients suffering from asthma or asthma-like symptoms, and isolated mast cells lacking endogenous PKC-θ protein expression.

Accordingly, in a first aspect, the invention provides a method for identifying a modulator of a PKC-θ protein. The method includes contacting a PKC-θ protein, or a functional fragment thereof, with a test agent; and determining if the test agent modulates the kinase activity of the PKC-θ protein, or the functional fragment thereof, wherein a change in the kinase activity of the PKC-θ protein, or the functional fragment thereof, in the presence of the test agent is indicative of a modulator of a PKC-θ protein. In certain embodiments, the determining step comprises comparing the kinase activity of the test agent relative to the absence of the test agent.

In some embodiments, the modulator of a PKC-θ protein that reduces the kinase activity is an inhibitor of the PKC-θ protein, or the functional fragment thereof. In some embodiments, the modulator of a PKC-θ protein that increases the kinase activity is an activator of the PKC-θ protein, or the functional fragment thereof. In some embodiments, the modulator of a PKC-θ protein reduces the kinase activity of the PKC-θ protein, a functional fragment thereof, by at least two-fold.

In certain embodiments, the PKC-θ protein is a full-length PKC-θ protein. In some embodiments, the PKC-θ protein is a functional variant of a full-length PKC-θ protein. In particular embodiments, the functional fragment is a PKC-θ kinase domain.

In some embodiments, the contacting step is effected by providing a reaction mixture of the PKC-θ protein, or the functional fragment thereof, and the test agent. In certain embodiments, the reaction mixture is in a buffer comprising a concentration of NaCl that is selected from the group consisting of 50 mM-100 mM, 100-150 mM, 150-200 mM, and 200-250 mM, and 250-300 mM. In particular embodiments, the concentration of NaCl is 250 mM.

In some embodiments, the modulator of a PKC-θ protein is useful for treating asthma in a mammal, such as a human. In some embodiments, the asthma is IgE-mediated asthma. In particular embodiments, the method further includes assessing the efficacy of the test agent in an in vitro or in vivo asthma model, where a test agent that shows an increased efficacy in the in vitro or in vivo asthma model as compared to a control agent is identified as being useful for treating asthma.

In some embodiments, the PKC-θ protein, or fragment thereof, is obtained from a prokaryotic cell, such as a bacterial cell (e.g., E. coli).

In some embodiments, the contacting step is effected in a cell.

In certain embodiments, the kinase activity of the PKC-θ protein, or the functional fragment thereof, is the autophosphorylation of the PKC-θ protein, or the functional fragment thereof. In some embodiments, the autophosphorylation of the PKC-θ protein, or the functional fragment thereof, occurs on an amino acid residue of SEQ ID NO: 1 selected from the group consisting of the serine residue at position 695, the serine residue at position 685, the threonine residue at position 538, and the threonine residue at position 536. In particular embodiments, the autophosphorylation occurs on the threonine residue at position 538 of SEQ ID NO: 1.

In some embodiments, the method includes contacting the PKC-θ protein, or the functional fragment thereof, with the test agent as well as a PKC-θ substrate. In certain embodiments, the kinase activity of the PKC-θ protein, or the functional fragment thereof, is the phosphorylation of the PKC-θ substrate. In some embodiments, the PKC-θ substrate comprises an R-X-X-S motif or an R-X-X-T motif, wherein R is arginine, X can be an unknown amino acid or can be any amino acid, S is serine, and T is threonine. For example, the PKC-θ substrate may have an amino acid sequence (based on the universal single letter amino acid code) selected from the group consisting of KKRFSFKKSFK (SEQ ID NO: 5), FARKGSLRQKN (SEQ ID NO: 6), FARKGSLRQ (SEQ ID NO: 15), KKRFSFKKSFK (SEQ ID NO: 16), QKRPSQRSKYL (SEQ ID NO: 17), KIQASFRGHMA (SEQ ID NO: 18), LSRTLSVAAKK (SEQ ID NO: 19), AKIQASFRGHM (SEQ ID NO: 20), VAKRESRGLKS (SEQ ID NO: 21), KAFRDTFRLLL (SEQ ID NO: 22), PKRPGSVHRTP (SEQ ID NO: 23), ATFKKTFKHLL (SEQ ID NO: 24), SPLRHSFQKQQ (SEQ ID NO: 25), KFRTPSFLKKS (SEQ ID NO: 26), IYRASYYRKGG (SEQ ID NO: 27), KTRRLSAFQQG (SEQ ID NO: 28), RGRSRSAPPNL (SEQ ID NO: 29), MYRRSYVFQT (SEQ ID NO: 30), QAWSKTTPRR1 (SEQ ID NO: 31), RGFLRSASLGR (SEQ ID NO: 32), ETKKQSFKQTG (SEQ ID NO: 33), DIKRLTPRFTL (SEQ ID NO: 34), APKRGSILSKP (SEQ ID NO: 35), MYHNSSQKRH (SEQ ID NO: 36), MRRSKSPADSA (SEQ ID NO: 37), TRSKGTLRYMS (SEQ ID NO: 38), LMRRNSVTPLA (SEQ ID NO: 39), ITRKRSGEAAV (SEQ ID NO: 40), EEPVLTLVDEA (SEQ ID NO: 41), SQKRPSQRHGS (SEQ ID NO: 42), KPFKLSGLSFK (SEQ ID NO: 43), AFRRTSLAGGG (SEQ ID NO: 44), ALGKRTAKYRW (SEQ ID NO: 45), VVRTDSLKGRR (SEQ ID NO: 46), KRRQISIRGIV (SEQ ID NO: 47), WPWQVSLRTRF (SEQ ID NO: 48), GTFRSSIRRLS (SEQ ID NO: 49), RVVGGSLRGAQ (SEQ ID NO: 50), LRQLRSPRRTQ (SEQ ID NO: 51), KTRKISQSAQT (SEQ ID NO: 52), NKRRATLPHPG (SEQ ID NO: 53), SYTRFSLARQV (SEQ ID NO: 0.54), NSRRPSRATWL (SEQ ID NO: 55), RLRRLTAREAA (SEQ ID NO: 56), NKRRGSVPILR (SEQ ID NO: 57), GKRRPSRLVAL (SEQ ID NO: 58), QKKRVSMILQS (SEQ ID NO: 59), and RLRRLTAREAA (SEQ ID NO: 60).

In some embodiments, the PKC-θ protein, or the functional fragment thereof, is in a cell, such as a mast cell or a CD4+ T cell.

In a further aspect, the invention provides a method for a method for identifying a modulator of a PKC-θ protein, comprising contacting a cell expressing a PKC-θ protein, or a functional fragment thereof, with a test agent and determining if the test agent reduces the amount of functional PKC-θ protein in the cell, wherein a test agent that reduces the amount of functional PKC-θ protein in the cell is identified as a modulator of a PKC-θ protein. In some embodiments, the modulator of a PKC-θ protein is useful for treating asthma in a mammal, such as a human. In some embodiments, the asthma is IgE-mediated asthma. In particular embodiments, the method further includes assessing the efficacy of the test agent in an in vitro or in vivo asthma model, where a test agent that shows an increased efficacy in the in vitro or in vivo asthma model as compared to a control agent is identified as being useful for treating asthma.

In some embodiments, the agent reduces expression of a nucleic acid molecule encoding the functional PKC-θ protein in the cell. In particular embodiments, the asthma is IgE-mediated asthma. In some embodiments, the mammal is a human. In certain embodiments, the functional PKC-θ protein is in a cell, such as a mast cell or a CD4+ T cell (e.g., a TH2 T cell).

In certain embodiments, the agent reduces the amount of an RNA encoding the functional PKC-θ protein. In some embodiments, the agent inhibits translation of an RNA encoding the functional PKC-θ protein.

In a further aspect, the invention provides a method for identifying an agent useful for treating asthma in a mammal, comprising contacting a nucleotide sequence encoding a reporter gene product operably linked to a PKC-θ promoter with a test agent and determining if the test agent reduces the production of the reporter gene product, wherein a test agent that reduces the production of the reporter gene product is identified as an agent useful for treating asthma.

In certain embodiments, the nucleotide sequence encoding a reporter gene product operably linked to a PKC-θ promoter is in a cell (e.g., a mast cell or CD4+ T cell). In some embodiments, the mast cell lacks expression of endogenous PKC-θ protein. In certain embodiments, the reporter gene product is luciferase, β-galactosidase, chloramphenicol acyltransferase, β-glucuronidase, alkaline phosphatase, or green fluorescent protein.

In a further aspect, the invention provides a method for identifying a modulator of a PKC-θ protein. The method comprises contacting a cell expressing PKC-θ protein, or a functional fragment thereof, with a test agent; and determining if the test agent reduces the autophosphorylation of the PKC-θ protein, or the functional fragment thereof, in the cell, wherein a test agent that reduces autophosphorylation of the the PKC-θ protein, or the functional fragment thereof, is identified as a modulator of a PKC-θ protein. In some embodiments, the determining step comprises comparing the kinase activity of the test agent relative to that in the absence of the test agent.

In some embodiments, the modulator of a PKC-θ protein that reduces the kinase activity is an inhibitor of the PKC-θ protein, or the functional fragment thereof. In some embodiments, the modulator of a PKC-θ protein that increases the kinase activity is an activator of the PKC-θ protein, or the functional fragment thereof. In some embodiments, the modulator of a PKC-θ protein reduces the kinase activity of the PKC-θ protein, or a functional fragment thereof, by at least two-fold.

In certain embodiments, the PKC-θ protein is a full-length PKC-θ protein. In some embodiments, the PKC-θ protein is a functional variant of a full-length PKC-θ protein. In particular embodiments, the functional fragment is a PKC-θ kinase domain.

In some embodiments, the modulator of a PKC-θ protein is useful for treating asthma in a mammal, such as a human. In some embodiments, the asthma is IgE-mediated asthma. In particular embodiments, the method further includes assessing the efficacy of the test agent in an in vitro or in vivo asthma model, where a test agent that shows an increased efficacy in the in vitro or in vivo asthma model as compared to a control agent is identified as being useful for treating asthma.

In some embodiments, the cell is a prokaryotic cell, such as a bacterial cell (e.g., E. coli).

In some embodiments, the autophosphorylation of the PKC-θ protein, or the functional fragment thereof, occurs on an amino acid residue of SEQ ID NO: 1 selected from the group consisting of the serine residue at position 695, the serine residue at position 685, the threonine residue at position 538, and the threonine residue at position 536.

In yet another aspect, the invention features a method for treating asthma, comprising administering to a mammal suffering from asthma or suffering from an asthma symptom an agent that reduces the kinase activity of PKC-θ protein, or a functional fragment thereof, or reduces the production of a functional PKC-θ protein. In some embodiments, the agent is administered with a pharmaceutically-acceptable carrier. In some embodiments, the carrier is in the form of an aerosol.

In certain embodiments of the inventive methods, the agent is administered by an intravenous, oral, transdermal, and/or intramuscular route. In particular embodiments, the agent is administered by inhalation. In some embodiments, the asthma is IgE-mediated asthma. In some embodiments, the agent is co-administered with a drug which may be an β-adrenergic agent, a theophylline compound, a corticosteroid, an anticholinergic, an antihistamine, a calcium channel blocker, a cromolyn sodium, or a combination thereof. In particular embodiments, the agent is an antibody that specifically binds to a PKC-θ protein or a fragment thereof. In some embodiments, the antibody is a polyclonal antibody. In some embodiments, the antibody is a monoclonal antibody.

In some embodiments, the test agent is a nucleic acid molecule. In certain embodiments, the nucleic acid molecule is a ribonucleic acid molecule. In some embodiments, the ribonucleic acid molecule comprises a nucleotide sequence that is complementary to a portion of the nucleotide sequence set forth in SEQ ID NO: 3.

In certain embodiments, the kinase activity of the PKC-θ protein, or the functional fragment thereof, is the autophosphorylation of the PKC-θ protein, or the functional fragment thereof. In some embodiments, the autophosphorylation of the PKC-θ protein, or the functional fragment thereof, occurs on an amino acid residue of SEQ ID NO: 1 selected from the group consisting of the serine residue at position 695, the serine residue at position 685, the threonine residue at position 538, and the threonine residue at position 536.

In certain embodiments, the kinase activity of the PKC-θ protein, or the functional fragment thereof, is the phosphorylation of a PKC-θ substrate. In some embodiments, the PKC-θ substrate comprises an R-X-X-S motif or an R-X-X-T motif, wherein R is arginine, X is either an unknown or any known amino acid, S is serine, and T is threonine. For example, the PKC-θ substrate may have an amino acid sequence (based on the universal single letter amino acid code) selected from the group consisting of KKRFSFKKSFK (SEQ ID NO: 5), FARKGSLRQKN (SEQ ID NO: 6), FARKGSLRQ (SEQ ID NO: 15), KKRFSFKKSFK (SEQ ID NO: 16), QKRPSQRSKYL (SEQ ID NO: 17), KIQASFRGHMA (SEQ ID NO: 18), LSRTLSVAAKK (SEQ ID NO: 19), AKIQASFRGHM (SEQ ID NO: 20), VAKRESRGLKS (SEQ ID NO: 21), KAFRDTFRLLL (SEQ ID NO: 22), PKRPGSVHRTP (SEQ ID NO: 23), ATFKKTFKHLL (SEQ ID NO: 24), SPLRHSFQKQQ (SEQ ID NO: 25), KFRTPSFLKKS (SEQ ID NO: 26), IYRASYYRKGG (SEQ ID NO: 27), KTRRLSAFQQG (SEQ ID NO: 28), RGRSRSAPPNL (SEQ ID NO: 29), MYRRSYVFQT (SEQ ID NO: 30), QAWSKTTPRR1 (SEQ ID NO: 31), RGFLRSASLGR (SEQ ID NO: 32), ETKKQSFKQTG (SEQ ID NO: 33), DIKRLTPRFTL (SEQ ID NO: 34), APKRGSILSKP (SEQ ID NO: 35), MYHNSSQKRH (SEQ ID NO: 36), MRRSKSPADSA (SEQ ID NO: 37), TRSKGTLRYMS (SEQ ID NO: 38), LMRRNSVTPLA (SEQ ID NO: 39), ITRKRSGEAAV (SEQ ID NO: 40), EEPVLTLVDEA (SEQ ID NO: 41), SQKRPSQRHGS (SEQ ID NO: 42), KPFKLSGLSFK (SEQ ID NO: 43), AFRRTSLAGGG (SEQ ID NO: 44), ALGKRTAKYRW (SEQ ID NO: 45), VVRTDSLKGRR (SEQ ID NO: 46), KRRQISIRGIV (SEQ ID NO: 47), WPWQVSLRTRF (SEQ ID NO: 48), GTFRSSIRRLS (SEQ ID NO: 49), RVVGGSLRGAQ (SEQ ID NO: 50), LRQLRSPRRTQ (SEQ ID NO: 51), KTRKISQSAQT (SEQ ID NO: 52), NKRRATLPHPG (SEQ ID NO: 53), SYTRFSLARQV (SEQ ID NO: 54), NSRRPSRATWL (SEQ ID NO: 55), RLRRLTAREAA (SEQ ID NO: 56), NKRRGSVPILR (SEQ ID NO: 57), GKRRPSRLVAL (SEQ ID NO: 58), QKKRVSMILQS (SEQ ID NO: 59), and RLRRLTAREAA (SEQ ID NO: 60).

In a further aspect, the invention provides an isolated mast cell lacking expression of endogenous PKC-θ protein. In some embodiments, the cell expresses exogenous PKC-θ protein or a fragment thereof.

These and other aspects, embodiments, and advantages of the present invention will be apparent from the descriptions herein.

### BRIEF DESCRIPTION OF THE FIGURES

FIGS. 1A-1C are photographic representations of Western blotting analyses depicting PKC-θ membrane translocation and inducible activation loop phosphorylation upon TCR co-stimulation of human T cells.

FIGS. 2A-2C are photographic (FIGS. 2A and 2C) and graphic (FIG. 2B) representations showing that autophosphorylation of the PKC-θ activation loop is required for kinase activity in cells.

FIGS. 3A-3D are schematic diagrams showing the characterization of the PKC-θ kinase domain (PKC-θ KD) autophosphorylation (FIG. 3A) and product ion spectra, as determined by mass spectrometry, of the peptide NFpSFMNPGMER (SEQ ID NO: 64; where “pS” indicates that the serine is phosphorylated; spanning positions 693-703) at m/z 705.52 (FIG. 3B), the peptide ALINpSMDQNMFR (SEQ ID NO: 65; spanning positions 681-692) at m/z 760.48 (FIG. 3C), and the peptide TNTFCGTPDYIAPEILLGQK (SEQ ID NO: 66; spanning positions 536-555) at m/z 1159.71 (FIG. 3D). Note that in FIG. 3D, the cysteine alkylated by iodoacetamide is indicated by #.

FIGS. 4A-4C are Western blotting analyses of E. coli lysates of the indicated PKC-θ KD protein and mutations, immunoblotting with anti-pT₅₃₈, PKC-θ (FIG. 4A), and anti-His to confirm equivalent expression (FIG. 4B), and a graph showing the in vitro lysate activity of the indicated PKC-θ KD protein and mutations (FIG. 4C).

FIGS. 5A-5D are a series of graphs showing the intercept replot versus 1/[Peptidel] at 100 mM NaCl (FIG. 5A); the slope replot versus 1/[Peptidel] at 100 mM NaCl (FIG. 5B); the intercept replot versus 1/[Peptidel] at 625 mM NaCl (FIG. 5C); and the slope replot versus 1/[Peptidel] at 625 NaCl (FIG. 5D).

FIGS. 6A-6C are a series of schematic diagrams showing various mechanisms by which the PKC-θ KD may behave kinetically. FIG. 6A shows a sequential ordered mechanism whereby ADP is the final product released; FIG. 6B shows a kinetic mechanism whereby ADP is the final product released, and FIG. 6C shows a random mechanism. In FIGS. 6A-6C, “E” stands for enzyme, “A” stands for substrate A, “B” stands for substrate B, “P” stands for product P, and “Q” is for product Q.

FIGS. 7A-7D show the solvent viscosity effects on k_{cat} (FIGS. 7A and 7C) and k_{cat}/Kₘ (FIGS. 7B and 7D) for PKC-θ KD. FIG. 7A shows the k_{cat} effect with varied peptide 1 with ATP held at 2.0 mM. FIG. 7B shows k_{cat}/Kₘ for peptide 1 with ATP held at 0.125 mM. FIG. 7C shows the k_{cat} effect with varied peptide 3 with ATP held at 2.0 mM. FIG. 7D shows the k_{cat}/Kₘ for Peptide 3 with ATP held at 2.0 mM. The open circle symbol (∘) indicates 100 mM NaCl in increasing sucrose; the open inverted triangle symbol (∇) indicates 250 mM NaCl in increasing sucrose; the closed circle symbol (●) indicates 100 mM NaCl in increasing Ficoll 400; and the closed inverted triangle symbol (▾) indicates 250 mM NaCl in increasing Ficoll 400. The dashed line in FIGS. 7A-7D indicates a slope of 1.

FIG. 8 is a schematic diagram showing different mechanisms by which inhibitory substrates can interfere with PKC-θ KD catalytic activity. In FIG. 8, “E” stands for enzyme, “A” stands for substrate A, “B” stands for substrate B, “P” stands for product P, and “Q” is for product Q.

FIGS. 9A-9B are representations of a peptide array scan identifying several peptide substrate sequences for PKC-θ (FIG. 9A) and the peptides identified a being phosphorylated by PKC-θ (FIG. 9B).

FIGS. 10A-10B are photographic representations of Western blotting analyses showing that the PKC-θ activation loop is inducibly phosphorylated upon IgE receptor cross-linking on bone marrow-derived mast cells (BMMC).

FIGS. 11A-11C are photographic representations of Western blotting analyses of the membrane fraction (FIG. 11A), the detergent-insoluble fraction (DI) (FIG. 11B), and whole cell extracts (WCE) (FIG. 11C) from IgE receptor cross-linked BMMC evidencing PKC-θ membrane translocation in IgE receptor-stimulated BMMC.

FIGS. 12A-12B are photographic representations of Western blotting analyses demonstrating that PKC-6 (FIG. 12A) and PKC-β (FIG. 12B) distribution is not significantly altered upon IgE receptor crosslinking on BMMC.

FIGS. 13A-13B are histological (FIG. 13A) and graphic (FIG. 13B) representations illustrating that BMMC from PKC-θ knockout mice contain fewer granules than BMMC from wild-type mice. Data from FIG. 13B show mean fluorescence intensity (MFI) of the cell as a function of time or as a function of DNP-BSA concentration.

FIGS. 14A-14B are graphic representations demonstrating that peritoneal mast cells from PKC-θ knockout mice have lower levels of cell surface IgE than cells from wild-type mice (FIG. 14A), but have similar levels of cell surface ckit (FIG. 14B). p values were determined by t-test.

FIGS. 15A-15C are graphic representations demonstrating that PKC-θ knockout mice have reduced levels of serum IgE (FIG. 15A) and IgG1 (FIG. 15B) compared to wild-type mice, but have increased levels of IgA (FIG. 15C). p values were determined by t-test.

FIGS. 16A-16C are graphic representations indicating that, following IgE receptor crosslinking, BMMC derived from PKC-θ knockout mice are deficient in production of the following cytokines: TNF-α (FIG. 16A), IL-13 (FIG. 16B), and IL-6 (FIG. 16C).

FIGS. 17A-17B are graphic representations showing that resting CD4+ T cells, TH1 cells, and TH2 cells from PKC-θ knockout mice showed reduced levels of IL-4 (FIG. 17A) and IL-5 (FIG. 17B) after culture in the absence of IL-2, and in the presence of 0.5 μg/ml anti-CD3.

FIG. 18 is a graphic representation evidencing that PKC-θ knockout mice do not have an increase in ear swelling in the passive cutaneous anaphylaxis (PCA) model described in Example 7 below in response to anti-IgE. Ear swelling was expressed as delta change from baseline. Statistical analyses were determined using the students unpaired t test. P values shown compare wild-type versus PKC-θ knockout animals.

FIG. 19 is a graphic representation demonstrating that PKC-θ knockout mice do not have an increase in ear swelling in the passive cutaneous anaphylaxis (PCA) model described below in the presence of exogenous IgE. Ear swelling was expressed as delta change from baseline. Statistical analyses were determined using the students unpaired t test. p values shown compare wild-type versus PKC-θ knockout animals.

FIGS. 20A-20D are representations of bar graphs showing that both TH1 and TH2 T cells from PKC-θ knockout mice show reduced proliferation to anti-CD3 stimulation (0.5 μg/ml) than both TH1 and TH2 T cells from PKC-θ wild-type mice. TH0, TH1, or TH2 cells from PKC-θ wild-type mice (light gray bars) or from PKC-θ knockout mice (dark gray bars) were additionally stimulated with anti-CD28 (FIG. 20A), anti-CD28 plus IL-2 (FIG. 20B), without anti-CD28 and without IL-2 (FIG. 20C), and with IL-2 in the absence of anti-CD28 (FIG. 20D).

FIGS. 21A-21D are representations of bar graphs showing that both TH1 and TH2 T cells from PKC-θ knockout mice show reduced proliferation to anti-CD3 stimulation (0.05 μg/ml) than both TH1 and TH2 T cells from PKC-θ wild-type mice. TH0, TH1, or TH2 cells from PKC-θ wild-type mice (light gray bars) or from PKC-θ knockout mice (dark gray bars) were additionally stimulated with anti-CD28 (FIG. 21A), anti-CD28 plus IL-2 (FIG. 21B), without anti-CD28 and without IL-2 (FIG. 21C), and with IL-2 in the absence of anti-CD28 (FIG. 21D).

### DESCRIPTION OF THE PREFERRED EMBODIMENTS

The invention is based on the discovery that agents that modulate protein kinase C theta (PKC-θ) or agents that modulate the amount of functional PKC-θ protein are useful for treating asthma. The novel findings presented here support the use of agents that reduce PKC-θ catalytic activity or reduce the amount of functional PKC-θ protein as agents for targeting mast cells in allergy and asthma.

For the purpose of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

The patent and scientific literature referred to herein establishes knowledge that is available to those of skill in the art. The issued U.S. patents, allowed applications, published applications (U.S. and foreign) and references, including GenBank database sequences, that are cited herein are incorporated by reference to the same extent as if each was specifically and individually indicated to be incorporated by reference.

PKC-θ is a member of the Ca⁺² independent novel class of PKCs. It is highly expressed in T cells and muscle. As described herein, the PKC-θ protein has been discovered to play a role in respiratory diseases, such as asthma, and to be associated with, for example, inducing the symptoms and/or complications associated with asthma, including, for example, atopic asthma, including IgE-mediated asthma; non-atopic asthma, occupational asthma, and drug-induced asthma. Based on the findings presented herein, the invention provides methods of identifying agents for treating asthma, and methods of treating asthma by administering to a mammal a therapeutically effective amount of an agent that modulates (e.g., by inhibiting or enhancing) PKC-θ production and/or kinase activity are provided. In addition, the invention provides isolated mast cells that lack endogenous PKC-θ protein expression.

In one aspect, the invention provides a method for identifying a modulator of a PKC-θ protein. The method includes contacting a PKC-θ protein, or a functional fragment thereof, with a test agent; and determining if the test agent inhibits the kinase activity of the PKC-θ protein, or the functional fragment thereof. A test agent that reduces the kinase activity of the PKC-θ protein, or the functional fragment thereof, is identified as a modulator of a PKC-θ protein.

As used herein, a “test agent” is a chemical (e.g., organic or inorganic), a small molecule compound, a nucleic acid molecule, a peptide, or a protein, such as a hormone, an antibody, and/or a portion thereof. By “modulator of PKC-θ protein” is meant an agent is able to modulate, either by increasing or decreasing, the kinase activity of a PKC-θ protein, or a functional fragment thereof, or is able to modulate the amount of functional PKC-θ protein (e.g., via transcription or translation). In some embodiments, the modulator of a PKC-θ protein that reduces the kinase activity is an inhibitor of the PKC-θ protein, or the functional fragment thereof. In some embodiments, the modulator of a PKC-θ protein that increases the kinase activity is an activator of the PKC-θ protein, or the functional fragment thereof.

In one form of the invention, the methods for identifying a modulator of a PKC-θ protein include contacting a PKC-θ protein, or a functional fragment thereof, with a test agent and detecting a change in the autophosphorylation of the a PKC-θ protein, or a functional fragment thereof (e.g., a change in the phosphorylation of the following residues of SEQ ID NO: 1: serine at position 695, serine at position 685, threonine at position 538, and threonine at position 536). In an alternate form, the methods include contacting a PKC-θ protein, or a functional fragment thereof, with a test agent and a substrate of PKC-θ, and detecting a change in the phosphorylation of the PKC-θ substrate. The test agent is one that is thought to be effective in modulating (i.e., inhibiting or increasing) the kinase activity of PKC-θ protein, or a functional fragment thereof, or the amount of functional PKC-θ protein (e.g., by changing the amount of RNA or DNA encoding functional PKC-θ protein). In certain embodiments, the modulator of a PKC-θ protein reduces the kinase activity of the PKC-θ protein, or the functional fragment thereof, by at least two-fold. In some embodiments, the modulator reduces the kinase activity of the PKC-θ protein, or the functional fragment thereof, by at least four-fold, or at least ten-fold. In some embodiments, the modulator abolishes the kinase activity of the PKC-θ protein, or the functional fragment thereof. PKC-θ protein kinase activity can be quantitated, for example, using standard techniques such as the in vitro kinase assays described below.

In another non-limiting embodiment of the invention, the amount of functional PKC-θ protein is reduced by the modulator of PKC-θ protein.

As used herein, “functional” means a PKC-θ protein, or a fragment thereof, that functions normally (e.g., has the same kinase activity as a wild-type PKC-θ protein). A determination of whether or not a PKC-θ protein, or fragment thereof, is functional may be easily made by the ordinarily skilled biologist. One non-limiting method for determining whether a PKC-θ protein, or fragment thereof, in question is functional is to compare the PKC-θ protein, or fragment thereof, in question with a wild-type PKC-θ protein or a wild-type PKC-θ fragment in a standard protein kinase assay (see, e.g., Ausubel et al., eds., Current Protocols in Molecular Biology, John Wiley & Sons, Inc., New York, N.Y. (1995, plus subsequent updates until 2003)) and the kinase assays described below in the examples.

One non-limiting example of a functional fragment of a PKC-θ protein is a PKC-θ kinase domain. As described in the examples below (particularly Example 3), the kinase domain of PKC-θ protein (also called “PKC-θ kinase domain” or simply “PKC-θ KD”) is surprisingly able to autophosphorylate. This is surprising, given that other enzymes in its class do not autophosphorylate. As used, the term “PKC-θ kinase domain” means the kinase domain of PKC-θ protein, which includes the portion of the protein spanning about amino acid residue 362 to about amino acid residue 706. In some embodiments, the PKC-θ KD of the invention has the amino acid sequence provided in SEQ ID NO: 61. In some embodiments, the PKC-θ KD of the invention has the amino acid sequence provided in SEQ ID NO: 62 (note that the first two N-terminal amino acid residues, methionine and glycine, of SEQ ID NO: 62, are convenient for expressing the PKC-θ KD fragment, but do not occur in the full length PKC-θ protein).

In some embodiments, the PKC-θ kinase domain of the invention is expressed in a prokaryotic cell, such as bacteria, such as E. coli. In some embodiments, the PKC-θ kinase domain is phosphorylated (e.g., by autophosphorylation) on one or more of the following amino acid residues: serine at position 695, serine at position 685, threonine at position 538, and threonine at position 536 of SEQ ID NO: 1.

In certain embodiments, the modulator of a PKC-θ protein reduces the amount of the functional PKC-θ protein by at least two-fold. In some embodiments, the modulator of a PKC-θ protein reduces the amount of the functional PKC-θ protein by at least four-fold. In some embodiments, the modulator of a PKC-θ protein reduces the amount of the functional PKC-θ protein by at least ten-fold. In some embodiments, the modulator of a PKC-θ protein abolishes the amount of the functional PKC-θ protein. Levels of functional PKC-θ protein can be quantitated, for example, using standard techniques, such as the Western blotting analyses described below.

In a further aspect, the invention provides another method for identifying a modulator of a PKC-θ protein, comprising contacting a cell comprising a functional PKC-θ protein, or a functional fragment thereof, with a test agent and determining if the test agent reduces the amount of functional PKC-θ protein, or functional fragment thereof, in the cell, wherein a test agent that reduces the amount of functional PKC-θ protein, or functional fragment thereof, in the cell is identified as a modulator of a PKC-θ protein. Such a modulator of a PKC-θ protein may act, for example, at the level of transcription or translation.

In certain embodiments, the modulator of a PKC-θ protein is useful for treating a respiratory disease in a mammal, such as a human. Respiratory diseases include, without limitation, asthma (e.g., allergic and nonallergic asthma); bronchitis (e.g., chronic bronchitis); chronic obstructive pulmonary disease (COPD) (e.g., emphysema); conditions involving airway inflammation, eosinophilia, fibrosis and excess mucus production, e.g., cystic fibrosis, pulmonary fibrosis, and allergic rhinitis.

In some embodiments, the modulator of a PKC-θ protein is useful for treating atopic diseases. “Atopic” refers to a group of diseases where there is often an inherited tendency to develop an allergic reaction. Non-limiting examples of atopic disorders include allergy, allergic rhinitis (hay fever, whose symptoms include itchy, runny, sneezing, or stuffy noses, and itchy eyes), atopic dermatitis (also known as eczema; a chronic disease that affects the skin), asthma, and hay fever.

In particular embodiments, the modulator of a PKC-θ protein is useful for treating asthma in a mammal, such as a human. “Asthma” as used herein, means a condition that is marked by continuous or paroxysmal labored breathing accompanied by wheezing, by a sense of constriction in the chest, and often by attacks of coughing or gasping. Any or all of these symptoms is included as an “asthma symptom”. As used herein, “asthma” includes, but is not limited to, non-allergic asthma (also called intrinsic or non-atopic asthma), allergic asthma (also called extrinsic or atopic asthma), combinations of non-allergic and allergic asthma, exercise-induced asthma (also called mixed asthma), drug-induced asthma, occupational asthma, and late stage asthma. Extrinsic or allergic asthma includes incidents caused by, or associated with, e.g., allergens, such as pollens, spores, grasses or weeds, pet danders, dust, mites, etc. As allergens and other irritants present themselves at varying points over the year, these types of incidents are also referred to as seasonal asthma. Also included in the group of extrinsic asthma is bronchial asthma and allergic bronchopulminary aspergillosis.

Asthma is a phenotypically heterogeneous disorder associated with intermittent respiratory disease symptoms such as, e.g., bronchial hyperresponsiveness and reversible airflow obstruction. Immunohistopathologic features of asthma include, e.g., denudation of airway epithelium, collagen deposition beneath the basement membrane; edema; mast cell activation; and inflammatory cell infiltration (e.g., by neutrophils, eosinophils, and lymphocytes). Airway inflammation can further contribute to airway hyperresponsiveness, airflow limitation, acute bronchoconstriction, mucus plug formation, airway wall remodeling, and other respiratory disease symptoms.

Asthma that can be treated or alleviated by the present methods include those caused by infectious agents, such as viruses (e.g., cold and flu viruses, respiratory syncytial virus (RSV), paramyxovirus, rhinovirus and influenza viruses. RSV, rhinovirus and influenza virus infections are common in children, and are one leading cause of respiratory tract illnesses in infants and young children. Children with viral bronchiolitis can develop chronic wheezing and asthma, which can be treated using the methods of the invention. Also included are the asthma conditions which may be brought about in some asthmatics by exercise and/or cold air. The methods of the inventin are useful for asthmas associated with smoke exposure (e.g., cigarette-induced and industrial smoke), as well as industrial and occupational exposures, such as smoke, ozone, noxious gases, sulfur dioxide, nitrous oxide, fumes, including isocyanates, from paint, plastics, polyurethanes, varnishes, etc., wood, plant or other organic dusts, etc. The methods are also useful for asthmatic incidents associated with food additives, preservatives or pharmacological agents. The methods of the invention are also useful for treating, inhibiting or alleviating the types of asthma referred to as silent asthma or cough variant asthma.

In addition, the methods of the invention are useful for the treatment and alleviation of asthma associated with gastroesophageal reflux (GERD), which can stimulate bronchoconstriction.

In some embodiments, the asthma is IgE-mediated asthma. In particular embodiments, the method further includes assessing the efficacy of the test agent in an in vitro or in vivo asthma model, wherein a test agent that shows an increased efficacy in the in vitro or in vivo asthma model as compared to a control agent is identified as being useful for treating asthma.

Various asthma models are known in the art. For example, Soler et al., J. Appl. Physiol. 70(2): 617-23 (1991) and Long et al., J. Appl. Physiol. 69(2): 584-590 (1990) describe a model for bronchoconstriction in sheep. Sheep are naturally sensitized to the roundworm parasite, Ascaris suum. Following inhalation challenge with Ascaris suum antigen, the animals undergo early- and late-phase bronchoconstriction responses, similar to the reaction of asthmatics upon exposure to sensitizing allergen. Ascaris challenge also induces airway hyperresponsiveness in the sheep, which is measured as an increase in lung resistance following provocation challenge with the cholinergic agonist, carbachol. The dose of carbachol required to elicit a given response decreases 24 hours following Ascaris challenge, and is an indication of airway hyperresponsiveness.

Bischof et al. (Clin. Exp. Allergy 33(3): 367-75 (2003)) describe a model for allergic asthma in sheep, where sheep immunized subcutaneously with solubilized house dust mite extract are subsequently given a single bronchial challenge with house dust mite. In this model, bronchoalveolar lavage (BAL) and peripheral blood leucocytes were collected before and after the brochial challenge of house dust mite for flow cytometry, and tissue samples were taken 48 hours post-challenge for histology and immunohistochemical analyses (Bischof et al., supra). A test agent thought to be a modulator of a PKC-θ protein, particularly one that is thought to be an inhibitor of the PKC-θ protein, can be administered to the sheep to assess its ability to reduce the number of BAL leukocytes following challenge as compared to the number of BAL leukocytes in sheep not administered a test agent of the invention.

Yet another well known asthma model is the non-human primate model of Ascaris—induced airway inflammation (see, e.g., Gundel et al., Clin. Exp. Allergy 22(1): 51-57 (1992)). Cynomolgus monkeys are naturally sensitized to the roundworm parasite, Ascaris suum, which acts as an allergen by inducing a strong IgE response. Upon intra-tracheal challenge with the antigen, the animals exhibit airway inflammation consisting primarily of eosinophils. This can be measured by counting leukocyte influx into the broncho-alveolar lavage fluid 24 hours following lung segmental allergen challenge.

Yet another non-limiting asthma model is the ovalbumin (OVA)-induced airway hyperresponsiveness in mice (see, e.g., Kips et al., Eur. Respir. J. 22(2): 374-382 (2003); Taube et al., Int. Arch. Allergy Immunol. 135(2): 173-186 (2004); and Reader et al., Am. J. Pathol. 162(6): 2069-2078 (2003)). In this model, mice are immunized with ovalbumin (OVA) in alum adjuvant, boosted, and then given an aerosol challenge with OVA. Upon challenge, the animals exhibit increased airway resistance, and infiltration of leukocytes into the bronchoalveolar lavage (BAL) fluid. In addition, serum cytokine levels increase, and lung histology shows tissue inflammation and mucous production.

Other non-limiting asthma models known in the art include the Ascaris suum antigen-induced asthma model in dogs and monkeys (see, e.g., Hirshman et al., J. Appl. Physiol. 49: 953-957 (1980); Mauser et al., Am. J. Respir. Crit. Care Med. 152(2): 467-472 (1995)).

In vitro asthma models are also known to the ordinarily skilled biologist. For example, for a T cell-targeted therapy, one non-limiting example is the inhibition of cytokine production by TH2 cells. The T cells can be stimulated in vitro antibodies to CD3 and CD28 to mimic TCR-mediated activation. This will induce cytokine production, which can be assayed in the supernatant 48 hours later. The key cytokines are IL-4 and IL-13. IL-13 especially is a major inducer of asthma pathogenesis in animal models (see, e.g., Wills-Karp M., Immunol Rev. 202: 175-190 (2004)).

Another non-limiting in vitro method for assessing the effect of a PKC-θ protein modulator on asthma is the inhibition of T cell proliferation or induction of the nuclear transcription factors NF-kB of NFAT in response to anti-CD3 and anti-CD28. T cell proliferation can be assayed, for example, by ³H-thymidine uptake (see methods, e.g., in Ausubel et al., supra). In response to T cell activation, NFAT or NF-kB undergo activation and nuclear translocation which can be assayed by Western blot from cell lysates.

PKC-θ protein inhibitors should also decrease TH2 responses in ovalbumin-immunized mice, which can be assayed as decreased production of ovalbumin-specific IgG1 or total IgE. The levels of these antibodies can be assayed by ELISA from the sera of mice.

As used herein, the PKC-θ protein of the invention may be from a human, and may have the amino acid sequence set forth in SEQ ID NO: 1 (GenBank Accession No: NM_{—}006257). In another embodiment, the PKC-θ protein of the invention may be from a mouse, and may have the amino acid sequence set forth in SEQ ID NO: 2 (GenBank Accession No: NM_{—}008859). PKC-θ proteins useful in the invention may also be encoded by a nucleotide sequence set forth in SEQ ID NO: 3 (human) (GenBank Accession No: NM_{—}006257) or SEQ ID NO: 4 (murine) (GenBank Accession No: NM_{—}008859). The sequences of additional PKC-θ proteins and nucleotide sequences encoding these proteins are available at GenBank Accession No: NM_{—}178075 (Niino et al., J. Biol. Chem. 276 (39): 36711-36717 (2001); (mouse)); GenBank Accession No. AF473820 (Normeman and Rohrer, Anim. Genet. 34 (1): 42-46 (2003); swine)).

As used herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide. The process of encoding a specific amino acid sequence may involve DNA sequences having one or more base changes (i.e., insertions, deletions, substitutions) that do not cause a change in the encoded amino acid, or which involve base changes which may alter one or more amino acids, but do not eliminate the functional properties of the polypeptide encoded by the DNA sequence.

The discovery that PKC-θ is associated with inducing the symptoms and/or complications of asthma renders the sequences of PKC-θ useful in methods of identifying agents of the invention. Such methods include assaying potential agents for the ability to modulate (e.g., inhibit or enhance) PKC-θ kinase activity. PKC-θ nucleic acid molecules (e.g., PKC-θ promoter sequences) and proteins useful in the assays of the invention include not only the genes and encoded polypeptides disclosed herein, but also variants thereof that have substantially the same activity as wild-type genes and polypeptides. “Variants” as used herein, includes polynucleotides or polypeptides containing one or more deletions, insertions or substitutions, as long as the variant retains substantially the same activity of the wild-type polynucleotide or polypeptide. With regard to polypeptides, deletion variants are contemplated to include fragments lacking portions of the polypeptide not essential for biological activity, and insertion variants are contemplated to include fusion polypeptides in which the wild-type polypeptide or fragment thereof has been fused to another polypeptide.

Thus, in certain embodiments, the PKC-θ protein of the invention is a functional variant of a full-length PKC-θ protein. It is therefore understood that the PKC-θ protein is not limited to being encoded by the nucleotide sequences set forth in SEQ ID NO: 3 or SEQ ID NO: 4. For example, nucleotide sequences encoding variant amino acid sequences, as discussed above, are within the scope of nucleotide sequences that encode PKC-θ. Modifications to a sequence, such as deletions, insertions or substitution in the sequence, which produce “silent” changes that do not substantially affect the functional properties of the PKC-θ protein are expressly contemplated herein. For example, it is understood that alterations in a nucleotide sequence which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue such as valine, leucine or isoleucine. Similarly, changes which result in the substitution of one negatively charged residue for another, such as aspartic acid for glutamic acid, or one positively charged residue for another, such as lysine for arginine, can also be expected to produce a biologically equivalent PKC-θ protein.

For use in the assays described herein, PKC-θ protein may be purchased commercially for various suppliers, such as Panvera (Madison, Wis.), or may be produced by genetic engineering and protein purification methods known to the skilled artisan. For example, a nucleotide sequence encoding a mammalian PKC-θ protein may be introduced into a desired host cell, cultivated, isolated and purified. Such a nucleotide sequence may first be inserted into an appropriate or otherwise desired recombinant expression vector. For example, the nucleotide sequence encoding a mammalian PKC-θ protein may be subcloned into the pcDNA3 expression vector and expressed in human 293 cells, as described in the examples below. Expression of the PKC-θ protein or PKC-θ kinase domain in prokaryotic cells is also contemplated. For example, as described in the examples below, the PKC-θ protein or PKC-θ kinase domain can be subcloned into a bacterial expression vector, such as pET16b (commercially available from, for example, EMD Biosciences/Merck Biosciences. San Diego, Calif.), and expressed in bacterial cells. A “vector”, as used herein and as known in the art, refers to a construct that includes genetic material designed to direct transformation of a targeted cell. A vector may contain multiple genetic elements positionally and sequentially oriented, i.e., operably linked with other necessary or desired elements such that the nucleic acid in a nucleic acid cassette can be transcribed and, if desired, translated in the transfected cell.

Recombinant expression vectors may be constructed by incorporating the above-recited nucleotide sequences into a vector according to methods well known to the skilled artisan and as described, for example, in Sambrook et al., Molecular Cloning: A Laboratory Manual, Cold Springs Harbor Laboratory, 2′ ed., Cold Springs Harbor, N.Y. (1989). Other references describing molecular biology and recombinant DNA techniques are also further explained in, for example, DNA Cloning 1: Core Techniques, (D. N. Glover et al., eds., IRL Press, 1995); DNA Cloning 2: Expression Systems, (B. D. Hames et al., eds., IRL Press, 1995); DNA Cloning 3: A Practical Approach, (D. N. Glover et al., eds., IRL Press, 1995); DNA Cloning 4: Mammalian Systems, (D. N. Glover et al., eds., IRL Press, 1995); Oligonucleotide Synthesis (M. J. Gait, ed., IRL Press, 1992); Nucleic Acid Hybridization: A Practical Approach, (S. J. Higgins and B. D. Hames, eds., IRL Press, 1991); Transcription and Translation: A Practical Approach, (S. J. Higgins & B. D. Hames, eds., IRL Press, 1996); R. I. Freshney, Culture of Animal Cells: A Manual of Basic Technique, 4^{th} Edition (Wiley-Liss, 1986); and B. Perbal, A Practical Guide To Molecular Cloning, 2^{nd} Edition, John Wiley & Sons, 1988); and Current Protocols in Molecular Biology (Ausubel et al., eds., John Wiley & Sons), which is regularly and periodically updated.

A wide variety of vectors are known that have use in the invention. Suitable vectors include plasmid vectors, viral vectors, including retrovirus vectors (e.g., see Miller et al., Methods of Enzymology 217: 581-599 (1993)), adenovirus vectors (e.g., see Erzurum et al. Nucleic Acids Res. 21: 1607-1612 (1993); Zabner et al., Nature Genetics 6: 75-83 (1994); and Davidson et al., Nature Genetics 3: 219-223 (1993)) adeno-associated virus vectors (e.g., see Flotte et al., Proc. Natl. Acad. Sci. USA 90: 10613-10617 (1993)) and herpes viral vectors (e.g., see Anderson et al., Cell Mol. Neurobiol. 13: 503-515 (1993)). The vectors may include other known genetic elements necessary or desirable for efficient expression of the nucleic acid in a specified host cell, including regulatory elements. For example, the vectors may include a promoter and any necessary enhancer sequences that cooperate with the promoter to achieve transcription of the gene. By “enhancer” is meant nucleotide sequence elements which can stimulate promoter activity in a cell, such as a eukaryotic host cell.

As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intron sequences may be of variable lengths, some nucleotide sequences may be operably linked but not contiguous.

Numerous art-known methods are available for introducing the nucleotide sequence encoding a PKC-θ protein, and which may be included in a recombinant expression vector, into a host cell. Such methods include, without limitation, mechanical methods, chemical methods, lipophilic methods and electroporation. Exemplary mechanical methods include, for example, microinjection and use of a gene gun with, for example, a gold particle substrate for the DNA to be introduced. Exemplary chemical methods include, for example, use of calcium phosphate or DEAE-Dextran. Exemplary lipophilic methods include use of liposomes and other cationic agents for lipid-mediated transfection. Such methods are well known to the art and many of such methods are described in, for example, Gene Transfer Methods: Introducing DNA into Living Cells and Organisms, (P. A. Norton and L. F. Steel, eds., Biotechniques Press, 2000); and Current Protocols in Molecular Biology (Ausubel et al., eds., John Wiley & Sons), which is regularly and periodically updated.

A wide variety of host cells may be utilized in the present invention to produce the desired quantities of PKC-θ protein, or functional fragment thereof, for use in, for example, the screening assays described herein. Such cells include eukaryotic and prokaryotic cells, including mammalian and bacterial cells known to the art. Numerous host cells are commercially available from the American Type Culture Collection, Manassas, Va.

The PKC-θ protein, or functional fragment thereof, may be isolated and purified by techniques well known to the skilled artisan, including chromatographic, electrophoretic and centrifugation techniques. Such methods are known to the art and can be found, for example, in Current Protocols in Protein Science, J. Wiley and Sons, New York, N.Y., Coligan et al. (Eds.) (2002); and Harris, E. L. V., and S. Angal in Protein Purification Applications: A Practical Approach, Oxford University Press, New York, N.Y. (1990).

To aid in the purification and detection of a recombinantly produced PKC-θ protein or functional fragment thereof, the PKC-θ protein or functional fragment thereof, may be engineered such that it is “tagged”. In the some examples below, the PKC-θ protein and PKC-θ kinase domain (a non-limiting example of a functional fragment of a PKC-θ protein) are tagged with a histidine tag. This allows the his-tagged protein to bind to Nickel-NTA, and thus be purified. In other examples below, the PKC-θ proteinis tagged with a hemagglutinin (HA) tag and expressed in 293 cells. Other non-limiting, commercially available tags that can be used to aid in the purification and/or detection of a PKC-θ protein (or a functional fragment thereof include, without limitation, the myc tag (binds to anti-myc tag antibodies), the GST tag (binds to glutathione-Sepharose), and the flu tag (binds to anti-flu tag antibodies).

To determine whether a test agent inhibits the kinase activity of a PKC-θ protein or functional fragment thereof, one non-limiting assay which may be employed is to contact the PKC-θ protein (or functional fragment thereof) with a test agent for a time period sufficient to inhibit the kinase activity of the PKC-θ protein. This time period may vary depending on the nature of the inhibitor and the PKC-θ protein or functional fragment thereof selected. Such times may be readily determined by the skilled artisan without undue experimentation. A non-limiting test agent of the invention is one that decreases the kinase activity of the PKC-θ protein (or functional fragment thereof), although test agents that inhibit PKC-θ by, for example, binding to a substrate of PKC-θ, or that inhibit the kinase activity of PKC-θ by some other mechanism, are also envisioned.

As described below, PKC-θ is inducibly phosphorylated on at least one of the following residues in BMMC upon IgE receptor cross-linking: the serine at position 695, serine at position 685, threonine at position 538, or threonine at position 536 of SEQ ID NO: 1. Thus, in a particular embodiment, a test agent may be determined to be an agent able to inhibit the kinase activity of PKC-θ (and thus useful for treating asthma) by its ability to inhibit the autophosphorylation of the PKC-θ protein. In some embodiments, the autophosphorylation of an amino acid residue of the activation loop of the PKC-θ protein is inhibited.

Numerous assays may be utilized to determine whether the test agent inhibits the kinase activity of the PKC-θ protein. As the PKC-θ protein is a kinase, such assays include measurement of the effect of the test agent on the ability of PKC-θ to autophosphorylate itself on the threonine residue at position 538 in the presence of a form of phosphate, such as adenosine triphosphate (ATP), or other form of phosphate which may be transferred to a PKC-θ substrate. Similarly, such an assay may measure the effect of the test agent on the ability of PKC-θ to phosphorylate a PKC-θ substrate in the presence of a form of phosphate. Radioactive-based assays and non-radioactive-based assays, including fluorescence-based assays, may be utilized. Radioactive-based assays measure, for example, incorporation of [γ-³²P]-ATP, into a PKC-θ substrate and measurement by liquid scintillation counting. Other assays employing in vitro substrate phosphorylation and antibody-based colorimetric detection or other methods of detection, are readily commercially available from a variety of sources including Promega (Madison, Wis.; Catalog Nos. V7470 and V5330), Calbiochem (San Diego, Calif.; Catalog Nos. 539484, 539490, 539491), Panvera Discovery Screening (Madison, Wis.; Catalog Nos. P2747 and P2748; which is a subsidiary of Invitrogen, Carlsbad, Calif.). Non-radioactive assays, which include phosphorylation of a substrate having the R-X-X-S/T consensus motif and measurement of the phosphorylated substrate by fluorescence polarization, include those sold by Panvera (Madison, Wis.).

In one non-limiting example, BMMC exposed to test agent and ³²P-ATP may be stimulated with anti-IgE receptor antibodies to crosslink the IgE receptor. Fifteen minutes following crosslinking, the cells may then lysed. Next, endogenous PKC-θ may be immunoprecipitated with commercially available antibodies (e.g., with the anti-PKC-θ antibody commercially available from Santa Cruz Biotechnology, Inc. (Santa Cruz, Calif.), which is described in the examples below), and resolved by SDS-PAGE analysis. PKC-θ from a BMMC treated with a test agent that inhibits PKC-θ autophosphorylation will show reduced phosphorylation (i.e., reduced incorporation of the ³²P-ATP) as compared to PKC-θ from untreated cells.

In an alternative of this example, BMMC are exposed to a test agent in the absence of ³²P-ATP. Fifteen minutes following anti-IgE receptor crosslinking, the cells are lysed, and endogenous PKC-θ immunoprecipitated and resolved by SDS-PAGE. The SDS-PAGE gel is then subjected to Western blotting analysis with anti-phosphothreonine antibodies (commercially available from, for example, Zymed Laboratories Inc., San Francisco, Calif.). PKC-θ from a BMMC treated with a test agent that inhibits PKC-θ autophosphorylation will show reduced phosphorylation (i.e., reduced incorporation of the ³²P-ATP) as compared to PKC-θ from untreated cells.

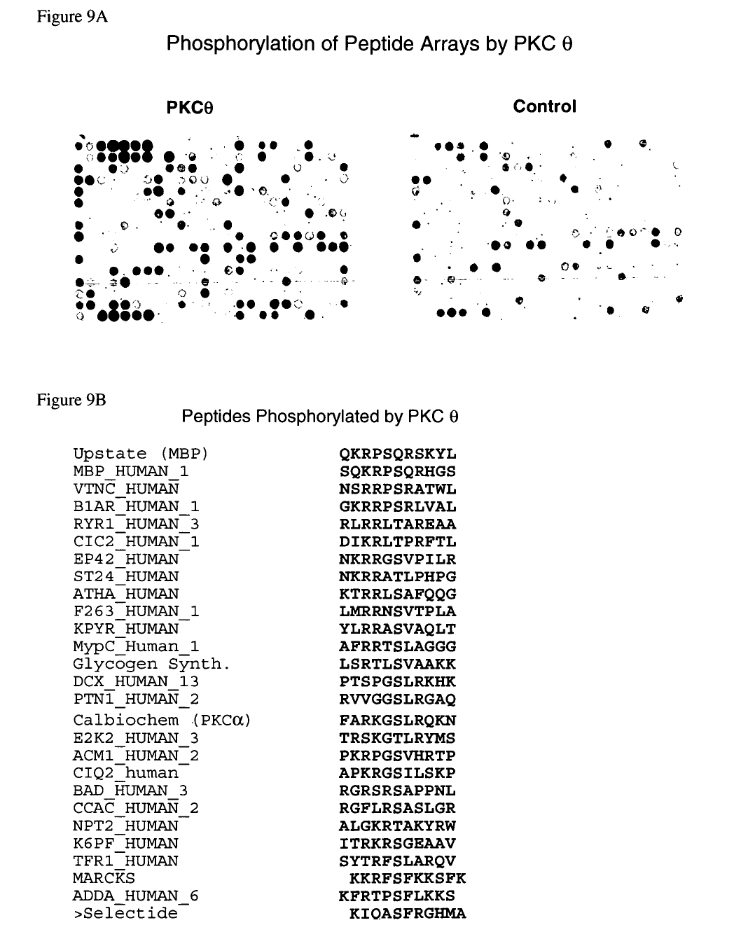

PKC-θ kinase activity can also be determined by its ability to phosphorylate a substrate. Thus, a wide variety of oligo-peptide and polypeptide substrates may be utilized in an assay to measure PKC-θ kinase activity. Peptides useful in the invention have the consensus R-X-X-S/T motif (wherein R is arginine, X is either an unknown or any known amino acid; S is serine and T is threonine). Other protein substrates include, without limitation, myristoylated alanine-rich C-kinase substrate (MARCKS) (amino acid sequence KKRFSFKKSFK (SEQ ID NO: 5), where the underlined serine residue is phosphorylated), PKC-α pseudo-substrate (amino acid sequence FARKGSLRQKN (SEQ ID NO: 6), where the underlined serine residue is phosphorylated). Using a peptide array technology, several potential substrates for PKC-θ have been identified that may contain sequences unique to the physiological substrate of PKC-θ and may be a therapeutic target with the same applications as PKC-θ (see FIG. 9B and Example 4 below). The substrates may have various modifications, as long as the substrates participate in a reaction catalyzed by PKC-θ.

Yet another method for measuring the kinase activity of PKC-θ is to measure its ability to autophosphorylate. In the examples described below, the kinase domain of PKC-θ was surprisingly found to be phosphorylated when expressed in bacterial cells. This phosphorylation was due to autophosphorylation because bacterial cells do not phosphorylate proteins. Thus, the invention also provides a method for identifying an agent useful for treating an immune disorder, in a mammal by contacting a cell expressing a PKC-θ protein (or a functional fragment thereof) with a test agent; and determining if the test agent reduces the autophosphorylation of the PKC-θ protein (or a functional fragment thereof) in the cell, wherein a test agent that reduces autophosphorylation of the PKC-θ protein (or a functional fragment thereof) is identified as an agent useful for treating the immune disorder. In some embodiments, the cell is a bacterial cell (e.g., E. coli). In some embodiments, the immune disorder is asthma.

In accordance with the invention, a cell can be made to express a PKC-θ protein, or a functional fragment thereof, by introducing into the cell a nucleotide sequence that encodes the PKC-θ protein or the functional fragment thereof. As discussed above, the nucleotide sequence is operably linked to regulatory sequences (e.g., promoter sequences and enhancers) that allow the cell to express the PKC-θ protein (or a functional fragment thereof). The ordinarily skilled artisan will understand that the types of regulatory sequences required to achieve expression of the nucleotide sequence encoding the PKC-θ protein (or a functional fragment thereof) will vary depending upon the type of cell into which the nucleotide sequence encoding the PKC-θ protein (or a functional fragment thereof) has been introduced. For example, if the cell is a bacterial cell, then regulatory sequences from a bacterial cell are preferably used. Regulatory sequences for numerous different types of cells (e.g., insect, mammalian, and bacterial) are well known in the art (see, e.g., Ausubel et al., Current Protocols in Molecular Biology, John Wiley & Sons, New York, N.Y., which is regularly and periodically updated).

In yet another aspect, the invention provides a method for identifying an agent useful for treating an immune disorder, such as asthma, in a mammal by contacting a functional PKC-θ protein or a PKC-θ kinase domain with a test agent; and determining if the test agent reduces the autophosphorylation of the functional PKC-θ protein or the PKC-θ kinase domain, wherein a test agent that reduces autophosphorylation of the functional PKC-θ protein or a PKC-θ kinase domain is identified as an agent useful for treating the immune disorder. In some embodiments, the contact is made in vitro.

In some embodiments, the contact of the functional PKC-θ protein or PKC-θ kinase domain with the test agent is made in a buffer. In some embodiments, the buffer has a high overall ionic strength relative to the ionic strength found inside a cell (approximately 100 mM NaCl). For example, in some embodiments, the buffer has an ionic strength of at least 100 mM. In some embodiments, the buffer has an ionic strength of at least 200 mM, or at least 250 mM.

In certain embodiments, the buffer in which the functional PKC-θ protein or PKC-θ kinase domain is contacted with the test agent contains NaCl. For example, the buffer may contain at least 50 mM NaCl (note that an additional salt (i.e., other than NaCl) may be present in the buffer). In some embodiments, the buffer contains at least 100 mM NaCl, or at least 150 mM NaCl, or at least 200 mM NaCl. In some embodiments, the buffer contains at least 250 mM NaCl. Of course, the ordinarily skilled artisan will understand that salts other than or in addition to NaCl can be used to obtain a buffer with high ionic strength. Some non-limiting examples of such salts include ammonium acetate, sodium acetate, and potassium chloride.

In accordance with the invention, an “immune disorder” is meant a disorder in which a cell of the immune system (e.g., a T cell, a B cell, a natural killer cell, a mast cell, a neutrophil, and a macrophage) does not function normally. In some embodiments, the immune disorder is asthma. Other immune disorders include, without limitation, autoimmune diseases (such as type I diabetes mellitus and rheumatoid arthritis), graft rejection, and respiratory diseases, such as allergy, in which immune cells play a role.

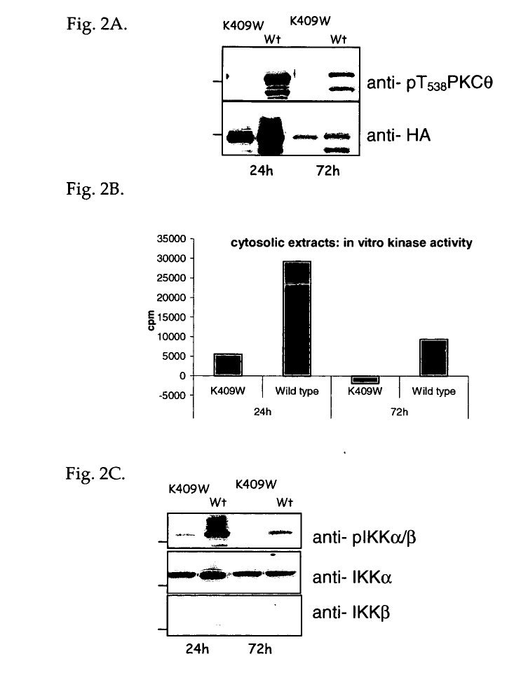

Thus, the invention provides methods for identifying agents that are useful in treating immune disorders, such as asthma, by identifying agents that modulate (e.g., decrease) the level of functional PKC-θ protein or agents that modulate (e.g., decrease) PKC-θ kinase activity. Agents that modulate (e.g., decrease) the production of functional PKC-θ protein or PKC-θ kinase activity include, without limitation, small molecule compounds, chemicals, nucleic acid molecules, peptides and proteins such as hormones, and antibodies. The agents may also include, for example, oligonucleotides or polynucleotides, such as antisense ribonucleic acid and small interfering RNAs (siRNA). The antisense nucleotide sequences and siRNAs typically include a nucleotide sequence that is complementary to, or is otherwise able to hybridize with, a portion of the target nucleotide sequence. In one non-limiting example, the antisense nucleotide sequence and/or siRNA hybridizes to the nucleotide sequence CAGAATATGTTCAGGAACTTTTCCTTCATGAACCCCG (SEQ ID NO: 7), which encodes the amino acid sequence QNMFRNFSFMNP (SEQ ID NO: 8), which corresponds to amino acid residues 688 to 699 that contains the serine residue at position 695 which is required for T538 autophosphorylation. In another non-limiting example, the antisense RNA and/or siRNA hybridizes to the nucleotide sequence GGAGATGCCAAGACGAATACCTTCTGTGGGACACCT (SEQ ID NO: 9), which encodes the amino acid sequence GDAKTNTFCGTP (SEQ ID NO: 10), which corresponds to amino acid residues 532 to 543 that contains the threonine residues at positions 536 and 538, at least one of which is required for kinase activity (see, e.g., FIGS. 2B and 2C). The antisense nucleotide sequences may have a length of about 20 nucleotides, but may range in length from about 20 to about 200 nucleotides, or may be the entire length of the gene target. The skilled artisan can select an appropriate target and an appropriate length of antisense nucleic acid in order to have the desired therapeutic effect by standard procedures known to the art, and as described, for example, in Methods in Enzymology, Antisense Technology, Parts A and B (Volumes 313 and 314) (M. Phillips, ed., Academic Press, 1999). Non-limiting examples of antisense molecules useful in the present invention are those described in Bennett et al., U.S. Pat. No. 6,190,869 (issued Feb. 20, 2001), hereby incorporated by reference.

RNA interference relates to sequence-specific, posttranscriptional gene silencing brought about by small, interfering double-stranded RNA fragments that are homologous to the silenced gene target (Lee, N. S. et al., Nature Biotech. 19: 500-505 (2002)). These siRNA could specifically target and eliminate natural mRNA molecules. Methods for inhibiting production of a protein utilizing siRNAs are well known to the art, and disclosed in, for example, PCT International Application Numbers WO 01/75164; WO 00/63364; WO 01/92513; WO 00/44895; and WO 99/32619.

Other agents that may be used to modulate (e.g., decrease) the production of functional PKC-θ protein or modulate (e.g., decrease) PKC-θ kinase activity include, without limitation, agents that block the translocation of PKC-θ to the cell surface membrane. Other agents that may be utilized include those found in the screening assays described herein.

Additional agents, or inhibitors or antagonists of PKC-θ, include, for example, antibodies and small molecules that specifically bind to PKC-θ protein or a portion of a PKC-θ protein. By “specifically binds” is meant that an antibody of the invention recognizes and binds to a PKC-θ protein (or a portion thereof) with a dissociation constant (K_{D}) of at least 10⁻⁵M, or with a K_{D} of at least 10⁻⁶ M, or with a K_{D} of at least 10⁻⁷ M, or with a K_{D} of at least 10⁻⁸ M, or with a K_{D} of at least 10⁻¹⁰ M. Standard methods for determining binding and binding affinity are well known. Accordingly, antibodies that specifically bind to PKC-θ protein are provided herein.

An antibody that specifically binds to the PKC-θ protein as used herein may be, without limitation, a polyclonal antibody, a monoclonal antibody, a chimeric antibody, a humanized antibody, a genetically engineered antibody, a bispecific antibody, antibody fragments (including but not limited to “Fv,” “F(ab′)₂,” “F(ab),” and “Dab”) and single chains representing the reactive portion of the antibody. Methods for production of each of the above antibody forms are well known to the art.

For instance, polyclonal antibodies may be obtained by injecting purified acid mammalian PKC-θ protein into various animals and isolating the antibodies produced in the blood serum, as more fully described, for example in Ausubel et al., Current Protocols in Molecular Biology, John Wiley & Sons, which is regularly and periodically updated. The antibodies may be monoclonal antibodies whose method of production is well known to the art.

Specific monoclonal antibodies may be obtained commercially or may otherwise be prepared by the technique of Kohler and Milstein, Eur. J. Immunol. 6: 511-519 (1976), and improvements or modifications thereof. Briefly, such methods include preparation of immortal cell lines capable of producing desired antibodies. The immortal cell lines may be produced by injecting the antigen of choice into an animal, such as a mouse, harvesting B cells from the animal's spleen and fusing the cells with myeloma cells to form a hybridoma. Single colonies may be selected and tested by routine procedures in the art for their ability to secrete high affinity antibody to the desired epitope.

Alternatively, antibodies may be recombinantly produced from expression libraries by various methods known to the art. For example, cDNA may be produced from ribonucleic acid (RNA) that has been isolated from lymphocytes, preferably from B lymphocytes and preferably from an animal injected with a desired antigen. The cDNA, such as that which encodes various immunoglobulin genes, may be amplified by the polymerase chain reaction (PCR) and cloned into an appropriate vector, such as a phage display vector. Such a vector may be added to a bacterial suspension, preferably one that includes E. coli, and bacteriophages or phage particles may be produced that display the corresponding antibody fragment linked to the surface of the phage particle. A sublibrary may be constructed by screening for phage particles that include the desired antibody by methods known to the art and including, for example, affinity purification techniques, such as panning. The sublibrary may then be utilized to isolate the antibodies from a desired cell type, such as bacterial cells, yeast cells or mammalian cells. Methods for producing recombinant antibodies as described herein, and modifications thereof, may be found, for example, in Griffiths, W. G. et al., Ann. Rev. Immunol. 12: 433-455 (1994); Marks, J. D. et al., J. Mol. Biol. 222: 581-597 (1991); Winter, G. and Milstein, C., Nature 349: 293-299 (1991); Hoogenboom, H. R. and Winter, G., J. Mol. Biol. 227: 381-388 (1992).

For use in the present invention, the PKC-θ protein may first be purified prior to being used for the generation of antibodies by techniques similarly well known to the skilled artisan, and previously discussed herein.

A further embodiment of the invention provides a non-limiting way to narrow the number of test agents by prescreening the test agents. For example, only those test agents having an ability to bind to the PKC-θ protein or the promoter directing PKC-θ gene expression may be used in the functional assays of the invention.

In a non-limiting example where the test agents are first screened for an ability to bind to the PKC-θ protein, purified PKC-θ protein can be isolated and used to screen test agents. For example, purified PKC-θ protein can be immobilized on a solid phase surface (e.g., on a sepharose bead or plastic), and test agents brought into contact with the purified immobilized PKC-θ protein. In an alternative example, following exposure of the PKC-θ protein with a test agent, antibodies directed against PKC-θ protein can be added and used to immunoprecipitate PKC-θ protein to determine if a test agent co-immunoprecipitated with the PKC-θ protein. Only those test agents that are able to bind to PKC-θ protein are next used in functional assays to determine if they can modulate (e.g., reduce) PKC-θ kinase activity or modulate (e.g., reduce) the amount of functional PKC-θ protein in a cell, such as a mast cell or a T cell (e.g., a TH1 or TH2 helper T cell).

In a non-limiting example where test agents are first screened for an ability to bind to the PKC-θ promoter, the PKC-θ promoter sequence can be immobilized, as in a DNA microchip array. Different test agents can then be screened for an ability to bind to the promoter. Only those test agents that are able to bind to the PKC-θ promoter are then used in functional assays to determine if they can modulate (e.g., reduce) the amount of functional PKC-θ protein in a cell, such as a mast cell or a T cell (e.g., a TH2 T cell).

In a further aspect, the invention provides a method for identifying an agent useful for treating asthma in a mammal (e.g., a human), comprising contacting a nucleotide sequence encoding a reporter gene product operably linked to a PKC-θ promoter with a test agent and determining if the test agent reduces the production of the reporter gene product, wherein a test agent that reduces the production of the reporter gene product is identified as agent useful for treating asthma. In certain embodiments, the nucleotide sequence encoding a reporter gene product operably linked to a PKC-θ promoter is in a cell (e.g., a mast cell or a T cell, such as a TH1 or TH2 helper T cell).

The nucleotide sequence of the PKC-θ promoter is determined by art-recognized methods. One nonlimiting example of such a method is to screen a genomic library (e.g., a YAC human genomic library) for the promoter sequence of interest using nucleotide sequence of PKC-θ as a probe, and then isolating the nucleotide sequence 5′ of where the probe bound. Another nonlimiting example of a method to determine the appropriate promoter sequence is to perform a Southern blotting analysis of the human genomic DNA by probing electrophoretically resolved human genomic DNA with a probe (e.g., a probe comprising the nucleotide sequence encoding human PKC-θ protein or a portion thereof) and then determining where the cDNA probe hybridizes. Upon determining the band to which the probe hybridizes, the band can be isolated (e.g., cut out of the gel) and subjected to sequence analysis. This allows detection of the nucleotide fragment 5′ of the nucleotides ATG (i.e., the start of transcription site). This nucleotide fragment is the promoter of PKC-O, and may be subjected to sequencing analysis. The nucleotide fragment may be between approximately 500 to 1000 nucleotides in length. Nucleotide sequences having at least about 70%, at least about 80% or at least about 90% identity to such sequences and that function as promoter, for example, to direct expression of a gene encoding a PKC-θ protein described herein, are also encompassed in the invention.

A wide variety of reporter genes may be operably linked to the PKC-θ promoter described above. Such genes may encode, for example, luciferase, β-galactosidase, chloramphenicol acetyltransferase, β-glucuronidase, alkaline phosphatase, and green fluorescent protein, or other reporter gene product known to the art.

In one form of the invention, the nucleotide sequence encoding a reporter gene that is operably linked to a PKC-θ promoter is introduced into a host cell. As discussed above, numerous host cells may be employed in the invention. Such a nucleotide sequence may first be inserted into an appropriate or otherwise desired recombinant expression vector as previously described herein.

The vectors in this form of the invention may include other known genetic elements necessary or desirable for expression of the reporter gene from the PKC-O promoter, including regulatory elements, in a mammalian cell. For example, the vectors may include any necessary enhancer sequences that cooperate with the promoter in vivo, for example, to achieve in vivo transcription of the reporter gene. The methods of introducing the nucleotide sequence into a host cell are identical to that previously described for producing the PKC-θ protein.

After contacting a nucleotide sequence encoding a reporter gene operably linked to a PKC-θ promoter with a test agent, it is determined if the test agent inhibits production of the reporter gene product. This endpoint may be determined by quantitating either the amount or activity of the reporter gene product. The method of quantitation will depend on the reporter gene that is used, but may involve use of an enzyme-linked immunosorbent assay with antibodies to the reporter gene product. Additionally, the assay may measure chemiluminescence, fluorescence, radioactive decay, etc. If the test agent inhibits production of the reporter gene product, it is classified as an agent for treating asthma.

Assays for determining the activity or amount of the reporter gene products described herein are known to the art and are discussed in, for example, Current Protocols in Molecular Biology (Ausubel et al., eds., John Wiley & Sons), which is regularly and periodically updated. Further descriptions of assays for the reporter gene products discussed herein may be found, for example, in the following publications: for luciferase, see Nguyen, V. T. et al., Anal. Biochem. 171: 404-408 (1988); for P-galactosidase, see, e.g., Martin, C. S. et al., Bioluminescence and Chemiluminescence: Molecular Reporting with Photons pp. 525-528 (J. W. Hastings et al., eds., John Wiley & Sons, 1997); Jain, V. K. and Magrath, I. T., Anal. Biochem. 199: 119-124 (1991); for β-galactosidase, β-glucuronidase and alkaline phosphatase see, for example, Bronstein, I. et al. Bioluminescence and Chemiluminescence: Fundamentals and Applied Aspects, pp. 20-23, (A. K. Campbell et al., eds., John Wiley & Sons, 1994); for chloramphenicol acetyltransferase, see Cullen, B., Methods. Enzymol. 152: 684 (1987); Gorman, C. et al. Mol. Cell. Biol. 2: 1044 (1982); Miner, J. N. et al., J. Virol. 62: 297-304 (1988); Sleigh, M. J., Anal. Biochem. 156: 251-256 (1986); Hruby, D. E. and Wilson, E. M., Methods Enzymol. 216: 369-376 (1992).

Small molecules that selectively inhibit PKC-θ activity are also therapeutic agents in treating asthma. Selectivity can be defined by about 20-fold greater IC50 for inhibiting PKC-θ over other PKC isoforms. (IC50 is defined as the concentration of inhibitor that results in fifty percent activity of the inhibitor target).

In another aspect of the invention, the invention provides methods for treating asthma that include administering to a mammal (e.g., a human) suffering from asthma or suffering from an asthma symptom a therapeutically effective amount of an agent that reduces the catalytic activity of PKC-θ or reduces the production of functional PKC-θ protein. In one embodiment, the mammal is a human. In some embodiments, the asthma is IgE-mediated asthma.

“Treatment”, “treating” or “treated” as used herein, means preventing, reducing or eliminating at least one symptom or complication of asthma. A “therapeutically effective amount” represents an amount of an agent that is capable of inhibiting or decreasing the production of a functional PKC-θ protein or capable of inhibiting or decreasing the kinase activity of a PKC-θ protein, and causes a clinically significant response. The clinically significant response includes, without limitation, an improvement in the condition treated or in the prevention of the condition. The particular dose of the agent administered according to this invention will, of course, be determined by the particular circumstances surrounding the case, including the agent administered, the particular asthma being treated and similar conditions. Asthma is treated by, for example, decreasing airway hyperresponsiveness, decreasing mucus hyperproduction, decreasing serum IgE levels or decreasing airway eosinophilia.

The agents may be administered to a mammal by a wide variety of routes, including enteral, parenteral and topical. For example, the agents may be administered orally, intranasally, by inhalation, intramuscularly, subcutaneously, intraperitonealy, intravascularly, intravenously, transdermally, subcutaneously, or any combination thereof.

The agents may be administered in a pharmaceutically-acceptable carrier. Pharmaceutically-acceptable carriers and their formulations are well-known and generally described in, for example, Remington: The Science and Practice of Pharmacy (20th Edition, ed. A. Gennaro (ed.), Lippincott, Williams & Wilkins, 2000). In some embodiments, the pharmaceutically-acceptable carrier is in the form of an aerosol. Any suitable pharmaceutically-acceptable carrier known in the art may be used. Carriers may be solid, liquid, or a mixture of a solid and a liquid. When present as a liquid or a mixture of a solid and a liquid, carriers that efficiently solubilize the agents are preferred. The carriers may take the form of capsules, tablets, pills, powders, lozenges, suspensions, emulsions or syrups or other known forms. The carriers may include substances that act as flavoring agents, lubricants, solubilizers, suspending agents, binders, stabilizers, tablet disintegrating agents and encapsulating materials. Solid or liquid carriers may be take the form of an aerosol to deliver the agents to their desired location, such as when used in a nebulizer for inhaling the agent.

Tablets for systemic oral administration may include excipients, as known in the art, such as calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with disintegrating agents, such as maize, starch or alginic acid, binding agents, such as gelatin, collagen or acacia and lubricating agents, such as magnesium stearate, stearic acid or talc. In powders, the carrier is a finely divided solid which is mixed with an effective amount of a finely divided inhibitor agent. In solutions, suspensions or syrups, an effective amount of the inhibitor agent is dissolved or suspended in a carrier such as sterile water, saline or an organic solvent, such as aqueous propylene glycol. Other compositions can be made by dispersing the inhibitor in an aqueous starch or sodium carboxymethyl cellulose solution or a suitable oil known to the art.

The agents are administered to a mammal in a therapeutically effective amount. Such an amount is effective in treating asthma or reducing asthma symptoms. This amount may vary, depending on the activity of the agent utilized, whether any other anti-asthmatic agent is co-administered and the nature of such anti-asthmatic agent, the nature of the asthma and the health of the patient. Although such amounts may be determined by the skilled artisan, typical therapeutically effective amounts include about 10 mg/kg/day to about 100 mg/kg/day. Of course, lower or higher dosages may be needed depending on the specific case. When the agents are combined with a carrier, they may be present in an amount of about 1 weight percent to about 99 weight percent, the remainder being composed of a pharmaceutically-acceptable carrier.

In certain embodiments, the agent or inhibitor of PKC-θ production or catalytic activity may be co-administered in, for example, a composition that includes one or more anti-asthmatic agents. Such agents are known to the art and include, for example, β-adrenergic agents, including isoproterenol, epinephrine, metaproterenol, and terbutaline; methylxanthines, including theophylline, aminophylline, and oxtriphylline; corticosteroids, including beclomethasone, betamethasone, hydrocortisone, and prednisone; anticholinergics, including atropine and ipratropium bromide; antihistamines, including terfenadine and astemizole; calcium channel blockers, including verapamil, nifedipine; and mast cell stabilizers, including cromolyn sodium and nedocromil sodium.

In some embodiments, the agent is a nucleic acid molecule. In certain embodiments, the nucleic acid molecule is a ribonucleic acid molecule. In some embodiments, the ribonucleic acid molecule comprises a nucleotide sequence that is complementary to a portion of the nucleotide sequence set forth in SEQ ID NO: 3. In certain embodiments, the agent reduces the amount of an RNA encoding the PKC-θ protein. In some embodiments, the agent inhibits translation of an RNA encoding the PKC-θ protein. In particular embodiments, the agent is an antibody (e.g., a polyclonal, monoclonal, humanized, or chimeric antibody) that specifically binds to PKC-θ protein, or a portion thereof.

In a further aspect, the invention features a cell which lacks expression of endogenous PKC-θ. In certain embodiments, the cell is a mast cell. Such a cell may be isolated from, for example, the PKC-θ knockout mouse described below (see also Sun et al., Nature 404: 402-407 (2000)). Methods for isolating mast cells are well known (see, e.g., the method described below). Such a cell lacking expression of endogenous PKC-θ protein may also be a human cell, in which the gene encoding PKC-θ had been deleted or mutated such that the cell no longer expresses endogenous PKC-θ.

A mast cell that lacks expression of endogenous PKC-θ protein is useful, for example, for testing whether a test agent is an agent useful for treating asthma. As described below in the examples, a hemagglutinin (HA)-tagged PKC-θ was expressed in 293 cells. HA-tagged PKC-θ can be expressed in mast cells and the activity and/or amount of the HA-tagged PKC-θ protein measured in these cells in the presence of a test agent. However, since mast cells express endogenous PKC-θ, some of the test agent may affect endogenous PKC-θ protein, thereby muting its effects on the HA-tagged protein. This muting will not occur in a mast cell lacking endogenous PKC-θ protein expression, and expressing HA-tagged PKC-θ protein. Moreover, such cells are useful for screening those test agents which affect HA-tagged PKC-θ differently than they affect wild-type PKC-θ.

In some embodiments, the cell expresses exogenous PKC-θ or a fragment thereof. Reference will now be made to specific examples illustrating the compositions and methods above. It is to be understood that the examples are provided to illustrate preferred embodiments and that no limitation to the scope of the invention is intended thereby.

### EXAMPLE 1

### PKC-θ Membrane Translocation and Activation Loop Phosphorylation upon TCR Co-Stimulation of Human T Cells

PKC-θ null (i.e., PKC-θ knockout) mice are viable, but mature T-cells are defective in proliferation, IL-2 production and activation of NF-KB (Sun et al., Nature 404: 402-407 (2000)). In human cultured mast cells (HCMC) it has been demonstrated that PKC kinase activity rapidly (<5 min) localizes to the membrane following IgE receptor crosslinking (Kimata et al., BBRC 3: 895-900 (1999)). Because PKC-θ plays a central role in TCR-mediated signaling and has a demonstrated effect in RBL-2H3 cells, a rat basophilic leukemia line (Liu et al., J. Leukocyte Biol. 69: 831-840 (2001)), the activation and function of PKC-θ in BMMC, peritoneal mast cells, and T cells was examined.

Following TCR stimulation, PKC-θ is rapidly translocated to the central region of the supramolecular activation complex where it remains for up to four hours (Huang et al., Proc. Natl. Acad. Sci. USA 99: 9369-9373 (2002)). To determine whether this translocation corresponded to a change in the phosphorylation of the PKC-θ protein, human T cells were purified and PKC-θ translocation and autophosphorylation analysed.

To purify T cells, mononuclear cell preparations were obtained from Biological Specialties (Colmar, Pa.). Cells were layered on Ficoll-Histopaque (commercially available from, e.g., Sigma Chemical Co., St. Louis, Mo.) and the buffy coat was collected following centrifugation. The cells were washed several times in PBS and cultured in RPMI/10% FCS at a density of 10⁶/ml. T cells were purified by negative selection (Dynal Biotech, Oslo, Norway). The purified T cells were stimulated with soluble anti-CD3ε (5 μg/ml crosslinked with 10 μg/ml anti-mIgG) and soluble anti-CD28 (5 μg/ml) for 0, 2, 10, 45, and 60 minutes (both anti-CD3ε and anti-CD28 commercially available from BD Biosciences, San Jose, Calif.).

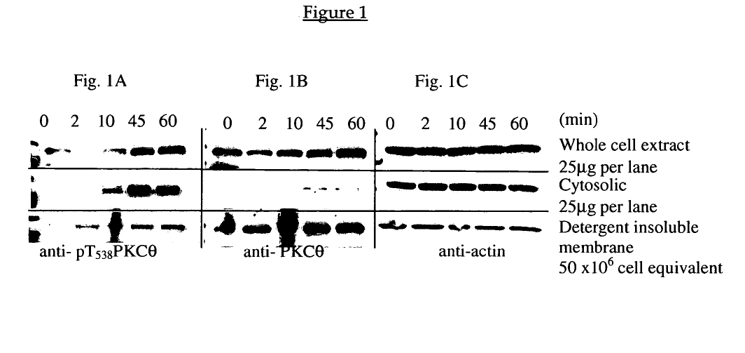

For analysis, the stimulated cells were collected by centrifugation and washed once in ice-cold PBS. Whole cell lysates were prepared by resuspending cell pellets in 100 μl of hypotonic lysis buffer [20 mM Tris-HCl, pH 7.5, 2 mM EDTA, 5 mM ethylene glycol-bis(B-amino-ethyl ether)-N,N,N′,N′-tetracetic acid (EGTA), 10 μg each of leupeptin and aprotinin per mL, protease cocktail and phosphatase inhibitors]. The cell suspension was sheared by passing through a 25 gauge needle 30 times, then centrifuged at 280×g for 7 minutes to precipitate the nuclei. The whole cell extract was cleared by high speed centrifugation (16,000×g) after saving an aliquot for analysis. The cytosolic extract was collected and the membrane pellets were washed once in the hypotonic lysis buffer and then resuspended in the same buffer with the addition of 1% NP-40 detergent for lysis on ice for 30 minutes. The detergent soluble membrane fraction was obtained by another high speed centrifugation step and remaining particulate fraction was the detergent insoluble membrane fraction (the DI fraction) containing membrane microdomains. This DI fraction was boiled in SDS-PAGE sample buffer for analysis. Subcellular protein fractions were analyzed by 4-20% SDS-PAGE, transferred to nitrocellulose and immunoblotted with anti-phosphoT₅₃₈ PKC-θ specific antibody (commercially available from Cell Signaling Technology, Inc. (Beverly, Mass.)) in 5% blotto/TBS-Tween 0.05% (see FIG. 1A).

Next, the nitrocellulose blot was stripped and reprobed with anti-PKC-θ E7 (Santa Cruz Biotechnology, Inc., Santa Cruz, Calif.) (see FIG. 1B). Finally, as shown in FIG. 1C, to show equal loading in all lanes, the blot was stripped again and then with anti-actin (commercially available from Santa Cruz Biotechnology, Inc.).

As FIG. 1A shows, PKC-θ is autophosphorylated in the activation loop of the kinase on the threonine residue at position 538 following TCR stimulation (via CD3 and CD28 stimulation). The autophosphorylation event coincides with the translocation of PKC-θ to the central region of the supramolecular activation complex (see FIG. 1B). As shown in FIG. 1C, approximately equal amounts of actin were found in all time treatments.

Thus, these results showed that the translocation of PKC-θ to the central region of the supramolecular activation complex corresponded with a concomitant inducible phosphorylation of the activation loop of the kinase on amino acid residue threonine 538.

### EXAMPLE 2

### PKC-θ Activation Loop Autophosphorylation is Required for Kinase Activity

As described in Example 1, PKC-θ membrane translocation corresponded with a concomitant inducible phosphorylation of the activation loop of the kinase on amino acid residue threonine 538 upon T cell receptor co-stimulation of human T cells. This activation loop phosphorylation has been reported as being required for kinase function (Liu et al., Biochemical Journal, 2002, 361-255-265). To confirm this report, a PKC-θ full length cDNA was subcloned with a C-terminal hemagglutinin (HA) epitope tag into the plasmid pcDNA3 (commercially available from Invitrogen), creating a C-terminal HA epitope tagged full length (WT) PKC-θ (nucleotide sequence SEQ ID NO: 11; amino acid sequence SEQ ID NO: 12). An HA-tagged kinase-dead PKC-θ was also generated by mutating the lysine at amino acid position 409 to tryptophan. This kinase-dead K409W mutation was generated by subcloning PCR products and confirmed by sequencing (nucleotide sequence SEQ ID NO: 13; amino acid sequence SEQ ID NO: 14), and was subcloned into the pcDNA3 expression vector. The human embryonic kidney 293 cells (commercially available from the American Type Culture Collection, Manassas, Va. were transiently transfected in duplicate with these expression constructs using lipids (using the Mirus TransIT-LT1 reagent commercially available from Mirus Corporation, Madison Wis.). Cells were harvested 24 or 72 hours following transfections for Western blot analysis and activity.

The harvested cells were lysed in hypotonic lysis conditions and nuclei were spun out (see more detailed methods in Example 1). The whole cell extracts from one replicate were run on SDS-PAGE and transferred to nitrocellulose and probed first with anti-phosphoT₅₃₈, PKC-θ specific antibody (Cell Signaling Technology), then stripped and reprobed with anti-HA antibody (Santa Cruz). As shown in FIG. 2A, the kinase dead full length PKC-θ protein was present (as determined by its staining with the anti-HA antibody), but was not phosphorylated on the threonine residue at position 538 (as determined by its lack of staining with the anti-pT₅₃₈PKC-θ antibody). Thus, as shown in the transfection experiments in FIG. 2A, while the wild-type kinase activation loop is effectively phosphorylated, the kinase dead version (generated by mutating the catalytic lysine at position 409 in the protein to a tryptophan, hence the name K409W) is not phosphorylated. Although evidence from other PKC isoforms indicated that the phosphorylation in the activation loop (i.e., the threonine at position 538) might have been attributed to the PDK-1 kinase based on evidence from other PKC isoforms, the results presented here indicate that the endogenous PDK-1 present in the human embryonic 293 kidney cells does not phosphorylate the PKC-θ activation loop. Activation loop autophosphorylation has also been proven by phospho blot analysis of bacterially expressed active kinase domain, and analysis of the purified kinase domain (data not shown).

Next, cytosolic extracts from the same replicate (i.e., those used in FIG. 2A) were analyzed for kinase activity in vitro using a peptide substrate. Cytosolic extracts were analyzed for kinase activity in vitro in 96 well plates with 5 μg protein each with a final concentration of 83 μM biotinylated peptide substrate (amino acid sequence FARKGSLRQ; SEQ ID NO: 15), 166 μM ATP, 0.5 μl of P³³ ATP (specific activity 3000 Ci/mmol, 10 mCi/ml), 84 ng/μl phophatidylserine, 8.4 ng/μl diacylglycerol in ADBII buffer (20 mM MOPS pH 7.2, 25 mM β-glyceroaldehyde, 1 mM sodium orthovanadate, 1 mM DTT, 1 mM CaCl₂) in a final volume of 30 μl for 30 minutes at room temperature. Kinase assays were stopped with buffer containing EDTA and transferred to streptavidin coated scintiplates for washing and radioactivity detection in a plate reader. Peptide only and kinase only reactions were subtracted from final counts as background.

As shown in FIG. 2B, the kinase dead full length PKC-θ protein had dramatically lower kinase activity at both 24 and 72 hours following transfection into human embryonic kidney 293 cells as compared to wild-type PKC-θ protein. Finally, the ability of wildtype and kinase dead PKC-θ to result in the phosphorylation of an endogenous substrate, IKK (IκBα kinase), was determined. To do this, cells from the duplicate sets were lysed in 1% NP-40 lysis buffer and the detergent insoluble membrane fractions were transferred to nitrocellulose. The nitrocellulose blots were probed first with anti-pIKKα/β, then stripped and reprobed with anti-IKKα, and finally stripped and reprobed with anti-IKKβ (all antibodies from Cell Signaling Technology). As shown in FIG. 2C, wild-type PKC-θ, but not kinase dead PKC-θ, resulted in the phosphorylation of IKK-β.

The results shown in FIGS. 2B and 2C demonstrate that the activation loop autophosphorylation (i.e., at the threonine at position 538) is required for PKC-θ activity and signaling, as shown by in vitro cell lysate kinase activity using a synthetic substrate (FIG. 2B), and phosphorylation of endogenous IKK (FIG. 2C). These results indicate that wild-type kinase induces IKK phosphorylation, whereas the kinase dead version fails to do so. These results identified PKC-θ activation loop autophosphorylation as a unique and novel mechanism for therapeutic modulation.

### EXAMPLE 3

### Mechanism of Catalysis of PKC-θ Kinase Domain

Studies were next performed to elucidate the mechanism of catalysis of the novel phosphorylated PKC-θ kinase domain (PKC-θ KD). To do this, catalytically active PKC-θ KD was expressed and purified for analysis of the phosphorylation sites. For these studies, the kinase domain of PKC-θ (PKC-θ KD; amino acid residues 362 to 706) was first expressed and purified. To do this, PKC-θ KD (amino acid residues 362 to 706) was cloned into a pET16b expression vector, introducing a hexa-histidine tag to the C-terminus. The amino acid sequence of the his-tagged PKC-θ KD is provided in SEQ ID NO: 63 (note that the N-terminal methionine and glycine residues in SEQ ID NO: 63 do not occur in full length PKC-0). The plasmid was used to transform E. coli strain BL21-DE3 for overexpression. A 10-liter cell culture at 37° C. of an optical density of 0.4, was induced with 0.1 mM IPTG at 25° C. for 3 hours before they were harvested and resuspended in buffer (25 mM Tris pH 8.0, 25 mM NaCl, 5 mM 2-mercaptoethanol, 5 mM imidazole, 50 μM ATP and protease inhibitors), and lysed using a microfluidizer.

The lysate was applied to 20 mL of Nickel-NTA resin for 1 hour at 4° C. The resin was subsequently poured as a chromatography column and washed extensively with the same buffer including 25 mM imidazole. Protein bound to the resin was eluted with 200 mM imidazole buffer. The protein was immediately loaded onto an anion exchanger HQ and the column was washed with 25 mM Tris pH 8.0, 25 mM NaCl, 5 mM DTT, 50 μM ATP before being resolved by the application of a linear gradient from 25 mM to 500 mM NaCl. Fractions containing PKC-θ KD were selected by SDS-PAGE, pooled, and diluted two-fold with 25 mM Tris pH 8.0, 5 mM DTT and loaded onto a heparin chromatography column. The flow-through was immediately applied to a hydroxy-apatite column and washed extensively with 25 mM Tris pH 8.0, 50 mM NaCl, 5 mM DTT. A linear gradient of sodium phosphate from 0 to 100 mM eluted the target protein. The protein was then sized as a monomer on a Superdex 200 size exclusion chromatography column, dialyzed overnight at 4° C. against 25 mM Tris pH 8.0, 50 mM NaCl, 5 mM DTT and concentrated.

Next, mass spectrometry analysis was performed. To do this, PKC-θ KD (in 50 mM Hepes pH 7.5, 5 mM MgCl₂, 5 mM DTT, 10% glycerol and 0.0025% Brij-35 at 0.25 μg/μl) was run on 10% Tricine gels (Invitrogen) and Comassie blue stained. The bands were excised and subjected to in-gel digestion with trypsin (Promega, Madison, Wis.) in a ProGest Investigator robot (Genomics Solutions, Ann Arbor, Mich.). The sample volume was reduced by SpeedVac and reconsituted with 0.1 M acetic acid to a final volume of approximately 30 μl. The peptides were then subjected to nanoLC/MS/MS analysis. Briefly, samples were injected onto a 75 μm×10 cm IntegraFrit column (New Objectives, Woburn, Mass.) that was packed with 10 μm C18 beads (YMC, Wilmington, N.C.). The HPLC gradient increased linearly from 4 to 60% solvent B (solvent A, 0.1 M acetic acid/1% ACN; solvent B, 0.1 M acetic acid/90% ACN) over 45 min with a flow-rate at 250 nL/min. Mass spectra were collected using a LCQ DECA XP ion trap mass spectrometer (ThermoFinnigan, San Jose, Calif.). The MS/MS data were searched against PKC-θ for differential phosphorylation modification on serine, threonine and tyrosine, using the Sequest algorithm (ThermoFinnigan, San Jose, Calif.).

To aid in the analysis of the mechanism of catalysis by PKC-θ KD, various mutations were made in the PKC-θ KD expression construct using site directed mutagenesis (using a kit commercially available from Stratagene, La Jolla, Calif.). The sequences of these mutations were confirmed by sequencing. The constructs were expressed as described above for the expression of wild-type PKC-θ KD, and equivalent amounts of E. coli lysates were analyzed by immunoblot and kinase assays after protein estimation by Bradford assay (commercially available from BioRad, Hercules, Calif.). Briefly, lysates were analyzed by 4-20% SDS-PAGE, transferred to nitrocellulose and immunoblotted with either the anti-pT₅₃₈ PKC-θ antibody commercially available from Cell Signaling Technology (Beverly, Mass.) or the anti-His antibody commercially available from Invitrogen (Carlsbad, Calif.) in 5% blotto/TBS-Tween 0.05%.

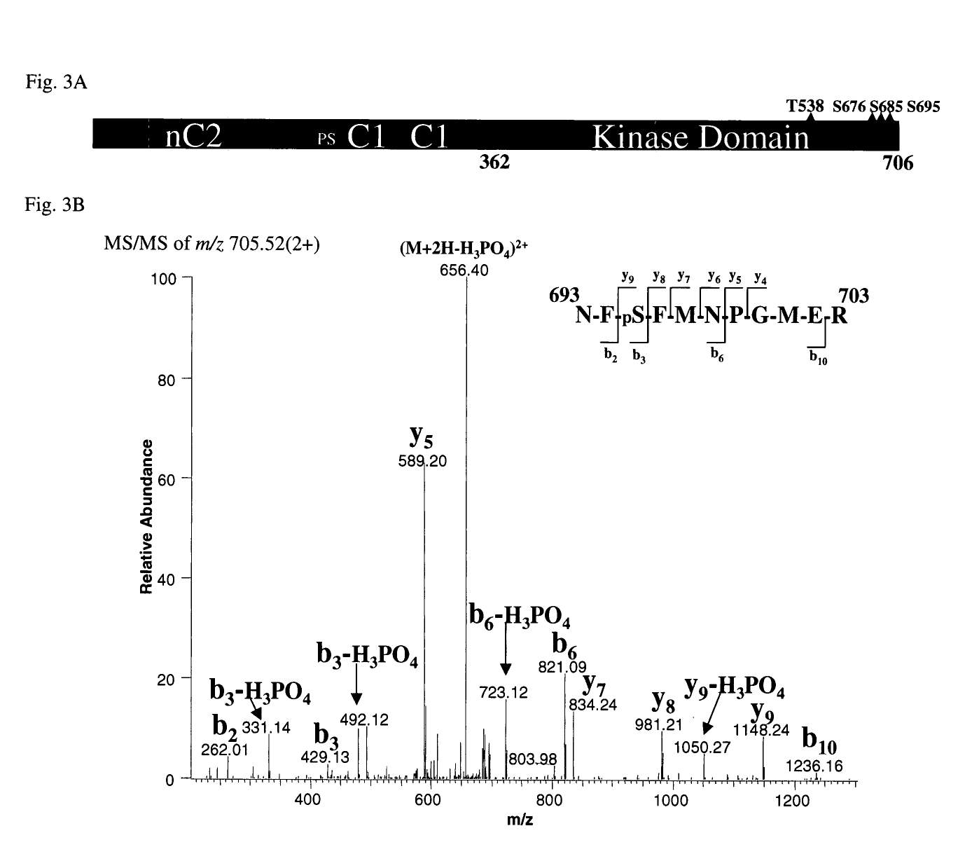

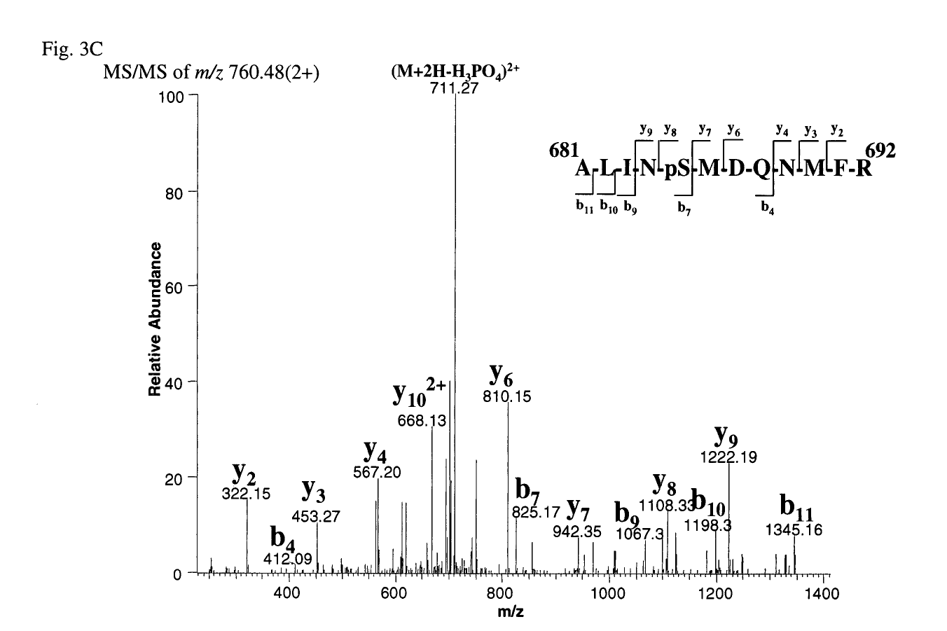

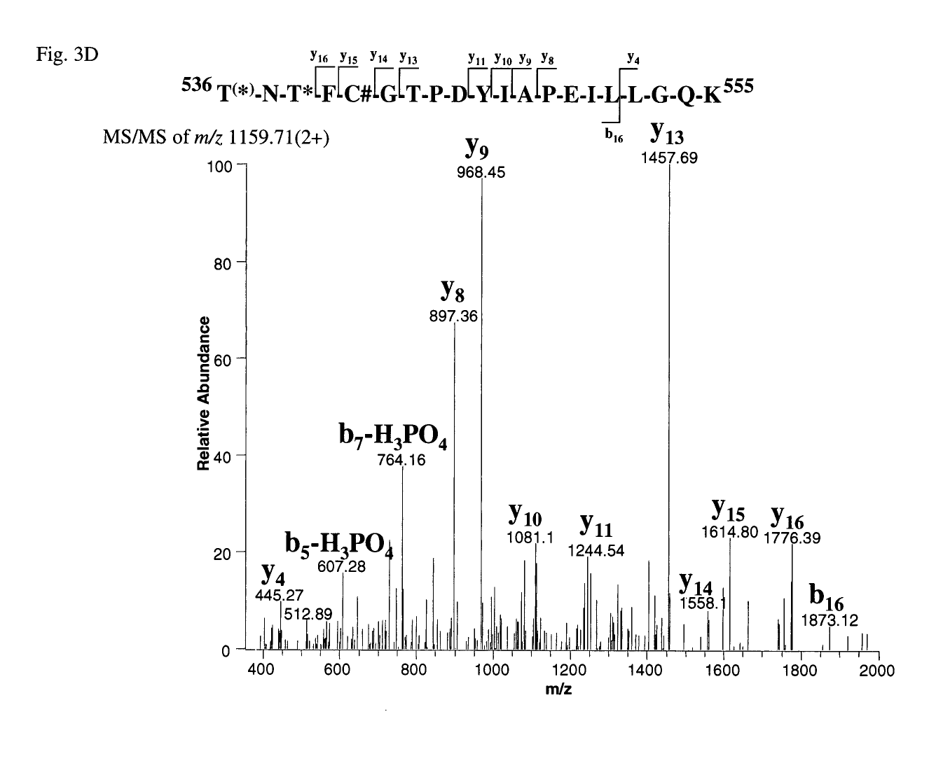

Mass spectrometry studies revealed that PKC-θ KD is phosphorylated. As PKC-θ KD expression was carried out in E. coli, where there are no serine-threonine kinases, the mass spectrometry finding is a consequence of autophosphorylation by the expressed kinase. The predicted mass based on the amino acid sequence is 41,615 Daltons, however, the molecular mass determination by ESI-MS was 42,092 Daltons and 42,173 Daltons (50% each species), which is indicative of autophosphorylation of 5 or 6 amino acids in E. coli. FIGS. 3A-3D are schematic diagrams showing the characterization of PKC-θ KD autophosphorylation. As FIG. 3A shows, the novel C2 domain is located at the protein's amino terminus, followed by two cofactor binding C1 domains, and then the carboxy-terminal kinase domain. The conserved phosphorylation sites (i.e., threonine at position 538, serine at position 676, serine at position 685, and serine at position 695) are indicated above the schematic of FIG. 3A, while the PKC-θ KD N-terminal and C-terminal amino acid residues (at positions 362 and 706, respectively) are indicated below the schematic.

In the mass spectrometry analysis, the m/z ratio is the mass/charge ratio of the peptide, and z (the charge) is 1. Thus, the m/z ratio gives the mass of the peptide fragment. Mass spectrometry product ion spectrum analysis indicated that that Ser₆₉₅ is the phosphorylation site.

Thus, FIG. 3B shows the product ion spectrum of the peptide NFpSFMNPGMER (spanning positions 693-703) at m/z 705.52, which confirmed that Ser₆₉₅ is the phosphorylation site. FIG. 3C shows the product ion spectrum of the peptide ALINpSMDQNMFR (spanning positions 681-692) at m/z 760.48, and indicated that Ser₆₉₅ is the phosphorylation site. FIG. 3D shows the product ion spectrum of the peptide TNTFCGTPDYIAPEILLGQK (spanning positions 536-555) at m/z 1159.71. The product ion spectrum of FIG. 3D indicated one phosphate on this peptide, and also indicated that the phosphorylation site is either Thr₅₃₆ or Thr₅₃₈. Note that the cysteine residue at position 540 (indicated by # in FIG. 3D) is alkylated by iodoacetamide.

Thus, the hydrophobic motif Ser₆₉₅ and the turn motif Ser₆₉₅ were identified as autophosphorylation sites (see FIGS. 3B and 3C, respectively). Mass spectrometry did not detect any phosphorylation at Ser₆₆₂ and Ser₆₅₇ turn motif residues. Based on homologies with other PKC turn motifs, Ser₆₇₆ is likely to be autophosphorylated, but this is not evident in these studies, as Ser₆₇₆ was not detected in a tryptic peptide.

These studies further revealed that either Thr₅₃₆ or Thr₅₃₈ in the activation loop is also autophosphorylated (see FIG. 3D). X-ray structure determination of the bacterially expressed PKC-θ KD confirmed the Thr₅₃₈ residue is phosphorylated (Xu et al., J. Biol. Chem. 279(48): 50401-50409 (2004)). This result is surprising given that it is in contrast to previous proposals that the activation loop is phosphorylated by PDK-1 (Balendran et al., FEBS Lett. 484: 217-223 (2000); LeGood et al., Science 281: 2042-2045 (1998)). Indeed, previous studies of the kinase-dead full length PKC-θ mutant K409W have shown that this molecule is not phosphorylated at Thr₅₃₈ (Liu et al., Biochem. J. 361: 255-265 (2002)). Using the HEK293 cell heterologous expression system as described in Example 2 above, the lack of Thr₅₃₈ phosphorylation of the K409W PKC-θ mutant in the cells was also observed (data not shown). This finding implies lack of Thr₅₃₈ phosphorylation due to the K409W kinase mutant's inability to autophosphorylate. Furthermore, the K409W PKC-θ molecule's abrogated Thr₅₃₈ phosphorylation correlated both with lack of in vitro cell lysate kinase activity, and endogenous IKKα/β phosphorylation (data not shown).

Because it was been previously suggested that the PKC-θ activation loop is phosphorylated by PDK-1 (Balendran et al., FEBS Lett. 484: 217-223 (2000); LeGood et al., Science 281: 2042-2045 (1998)), the results of mass spectrometry analysis indicating that either Thr₅₃₆ or Thr₅₃₈ of the bacterially expressed PKC-θ KD is autophosphorylated are surprising (see FIGS. 3B-3D). This is in part explained by the x-ray structure, that reveals the phosphorylated Thr₅₃₈ in hydrogen bond interactions with the side chain of the preceding Thr₅₃₆ (Xu et al., J. Biol. Chem. 279(48): 50401-50409 (2004)). This interaction likely further stabilizes the interactions within the activation loop and with the αC-helix, both of which have relevance in catalysis (Johnson et al., Cell 85: 149-158 (1996)).

Previous studies have suggested a catalytic competent conformation for PKC-θ wherein the activation loop is constitutively phosphorylated (Newton, A. C., Biochemical Journal. 370: 361-371 (2003)). PDK-1 phosphorylates PKCs and other AGC family kinases at the kinase domain activation loop, as a required modification that precedes autophosphorylation occurring at conserved sites on the hydrophobic and turn motifs (Newton, A. C., Biochemical Journal. 370: 361-371 (2003); Balendran et al., FEBS Lett. 484: 217-223 (2000)). The results presented herein reveal that in contrast to the prevailing hypothesis, PKC-θ is uniquely capable of autophosphorylation. The findings presented herein on the characterization of the PKC-θ KD present evidence to support that in addition to the hydrophobic and turn motifs within the kinase domain, the activation loop is also autophosphorylated (see FIGS. 3B-3D). These studies do not rule out the possibility that in cells PDK-1 can phosphorylate the PKC-θ activation loop. However, in contrast to bacterially expressed PKC-θ (Smith et al., J. Biol. Chem. 277: 45866-45873 (2002)), the findings presented in this Example show that PKC-θ KD is capable of autophosphorylation at the PKC-θ activation loop, and therefore, does not have an obligatory PDK-1 phosphorylation requirement.

The mass spectrometry data shows that the bacterially expressed PKC-θ KD is autophosphorylated at 5 or 6 amino acid residues. The phosphorylation sites identified in these experiments include hydrophobic motif Ser₆₉₅, turn motif Ser₆₈₅, and activation loop Thr₅₃₈ or Thr₅₃₆ Turn motif Ser₆₇₆ was not detected in a tryptic peptide, though is likely also phosphorylated based on sequence homology. Ser₆₉₅ is a newly identified autophosphorylation site in the turn motif. Finally, in addition to the above identified phosphorylation sites, at least 2 additional amino acid residues are autophosphorylated but not detected by these techniques.

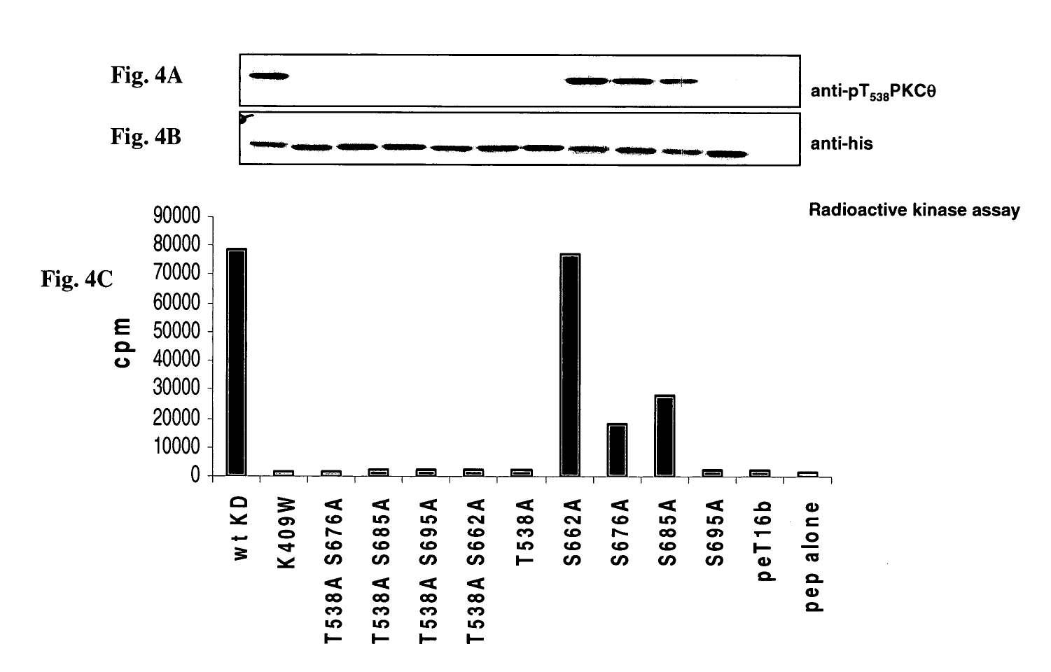

The amino acid residue Thr₅₃₈ in the activation loop is required for kinase activity (Liu et al., Biochem. J. 361: 255-265 (2002)). Accordingly, several phosphorylation site point mutations within the kinase domain were examined for their effects on activation loop Thr₅₃₈ autophosphorylation. To do this, E. coli lysates of PKC-θ KD protein and various mutations were assayed by Western blotting analysis using an anti-pT₅₃₈ PKC-θ antibody. As shown on FIG. 4A, only the wild-type PKC-θ KD protein and three mutant fragments tested were phosphorylated on the tyrosine at position 538. Equal loading of the lanes was determined by stripping the blot and reprobing with staining with an anti-His antibody (see FIG. 4B). Fractions of these E. coli lysates were also subjected to lysate kinase assays. These kinase assays were performed with a final concentration of 83 μM biotinylated peptide substrate (FARKGSLFQ), 166 μM ATP, 0.5 μl of P³³ ATP (specific activity 3000 Ci/mmol, 10 mCi/ml), 84 ng/μl phophatidylserine, 8.4 ng/l diacylglycerol in 20 mM MOPS pH 7.2, 25 mM β-glycerophosphate, 1 mM DTT, 1 mM CaCl₂, in 30 μl for 30 minutes at room temperature. Five to ten μl of the reaction was spotted on phosphocellulose paper, which was then washed three times in 0.75% phosphoric acid and once in acetone. Scintillation cocktail was added to the phosphocellulose paper and bound radioactivity was detected with a scintillation counter. As FIG. 4C shows, of the various PKC-θ KD mutants tested, only the wild-type PKC-θ KD protein and three mutant fragments there were phosphorylated on threonine 538 showed activity in an in vitro lysate kinase activity assay. Indeed, the lysate kinase activity correlates with the extent of phosphorylated threonine 538 (pThr₅₃₈) detected in the lysate for each of the expressed mutants (compare FIGS. 4A and 4C).

The serine at position 695 (Ser₆₉₅) in the C-terminal hydrophobic motif of PKC-θ KD is also required for optimal activation loop autophosphorylation, as evidenced by the significantly reduced signal in the anti-pT₅₃₈ Western blot panel (see the S695A mutant (i.e., serine at position 695 mutated to alanine) in FIG. 4A). Thus, the serine at position 695 is obligatory for PKC-θ KD kinase activity, as demonstrated by the lack of kinase activity of the S695A mutant (see S695A mutant in FIG. 4C), much like the inactive and kinase-dead mutations T538A and K409W, respectively (see FIGS. 4A and 4C). In contrast, turn motif residue Ser₆₆₂ is dispensable for both activity and Thr₅₃₈ autophosphorylation (see the S662A mutant in FIG. 4A), while the turn motif residues Ser₆₇₆ and Ser₆₉₅ have a partial impact (see S676A and S685A mutants in FIG. 4A).

Thus, mutation analysis demonstrated that both Ser₆₇₆ and Ser₆₈₅ in the conserved turn motif partially impact kinase function of the PKC-θ KD (see FIGS. 4A and 4C). It has been reported previously that the S676A mutation in the full length kinase does not affect kinase activity, while the S695A mutation in the full length molecule reduced kinase activity by 80% (Liu et al., Biochem. J. 361: 255-265 (2002)). The residual activity of the S695A reported in full length PKC-θ, is consistent with a modest phospho-Thr538 signal observed here for the S695A in the kinase domain context (see FIG. 4C, the S695A mutant). This suggests that Ser₆₉₅ mutation results in loss of optimal Thr₅₃₈ autophosphorylation, consequently resulting in attenuation of kinase activity. This feature is also unique to PKC-θ among other PKC isoforms. In the case of PKC-θ, it is likely that Ser₆₉₅ and Thr₅₃₈ autophosphorylation are somewhat interdependent. A PKC molecule phosphoryated at the activation loop, is described as a “catalytic competent conformation” that exists prior to cofactor binding, autophosphorylation, and substrate catalysis steps (Newton, A. C., Biochemical Journal. 370: 361-371 (2003)). For optimal PKC-θ KD kinase function it is likely that autophosphorylation of both activation loop and hydrophobic motif contributes to the PKC-θ “catalytic competent conformation”.

Having established the phosphorylation site relationship of the expressed active PKC-θ KD, detailed enzyme mechanism studies were next undertaken to examine the kinase catalytic reaction. The peptide substrates investigated for use in determining the kinetic mechanism of PKC-θ are shown in Table I.

TABLE I
Peptides used in assays with PKC-θ KD

| Peptide | sequence | | source | pI |
|---|---|---|---|---|
| 1 | FARKGSLRQ | substrate | pseudo-substrate | 12.01 |
| | | | PKC alpha | |
| 2 | RFARKGSLRQKNV | substrate | pseudo-substrate | 12.31 |
| | | | PKC alpha | |
| 3 | LKRSLSEM | substrate | Serum Response | 8.75 |
| | | | Factor | |
| 4 | RTPKLARQASIELPSM | substrate | lymphocyte- | 10.84 |
| | | | specific | |
| | | | protein 1 | |
| 5 | FARKGALRQ | inhibitor | pseudo-substrate | 12.01 |
| | | | PKC alpha | |

Peptide 1 and peptide 2 are substrates derived from the pseudosubstrate region of PKC-α. Peptide 3 and peptide 4 are derived from the phosphorylation site in serum response factor (Heidenreich et al., J. Biol. Chem. 274: 14434-14443 (1999)) and the phosphorylation site in lymphocyte-specific protein-1, respectively (Huang et al., J. Biol. Chem. 272: 17-19 (1997)).

For enzyme kinetic assays, ATP, ATPγS, Ficoll-400, sucrose, ATP, ADP, phosphoenolpyruvate (PEP), NADH, pyruvate kinase (PK), lactate dehydrogenase (LDH), AMP-PNP, acetonitrile, and the buffer HEPES were purchased from Sigma Chemical Co. (St. Louis, Mo.). Peptide substrates, inhibitors and phosphorylated substrate peptides were purchased from AnaSpec (San Jose, Calif.), SynPep (Dublin, Calif.) or Open Biosystems (Huntsville, Ala.). The enzymatic activity was determined at 25° C. using the coupled PK/LDH assay, followed spectrophotometrically at 340 nm on a Molecular Devices platereader. The standard reaction, except where indicated, was carried out in 25 mM HEPES pH 7.5, 10 mM MgCl₂, 2 mM DTT, 0.008% TritonX100, 100 mM NaCl, 20 units PK, 30 units LDH, 0.25 mM NADH, and 2 mM PEP, in a final volume of 0.080 mL. The PKC-θ KD concentration varied between 0.156 μg/ml to 0.312 μg/ml.

Next, solvent viscosity studies were performed. Steady state kinetic parameters were determined in the buffer described above for the enzyme kinetics assays containing varied sucrose (0-35%) or Ficoll 400 (0-8%). Relative solvent viscosities (η^{ref}) were determined in triplicate relative to 25 mM HEPES pH 7.5, 10 mM MgCl₂, 2 mM DTT and 100 mM NaCl at 25° C. using an Ostwald viscometer. Buffer with no viscogen is indicated with a superscript of 0. The coupling enzyme system was unaffected by the presence of these viscogens. Thio effect studies with ATPγS and product inhibition studies with ADP were analyzed on a Hewlett Packard series 1100 HPLC using a Phenomenex Auga 5 m C18 124 A⁰ 50 mm×4.60 mM column (00B-4299-E0). Phosphorylated peptide was separated from non-phosphorylated peptide using a gradient of 0% to 100% 20 mM phosphate pH 8.8/acetonitrile (50/50). The fluorescein-labeled peptide was detected by excitation at 485 nm and monitoring the fluorescence emission at 530 nm.

Substrate kinetics were next determined. To do this, data were fit to equation 1 for normal Michaelis-Menten kinetics or equation 2 for substrate inhibition:

$$\begin{matrix}v = \frac{{V}_{max} [ S ]}{{K}_{m} + [ S ]} & ( 1 ) \\ v = \frac{{V}_{max} [ S ]}{Km + [ S ] + \frac{{[ S ]}^{2}}{{K}_{i}}} & ( 2 )\end{matrix}$$

where S is the substrate, Vₘₐₓ is the maximum enzyme velocity, Kₘ is the Michaelis constant and Kᵢ is the inhibition constant for substrate inhibition (Adams, J. A., Biochemistry 42: 601-607 (2003)). The initial rates obtained at various fixed concentration of peptides and ATP and were fitted to the equations listed below:

$$\begin{matrix}v = \frac{{V}_{max} [ A ] [ B ]}{{K}_{ia} {K}_{b} + {K}_{b} [ A ] + {K}_{a} [ B ] + [ A ] [ B ]} & ( 3 ) \\ v = \frac{{V}_{max} [ A ] [ B ]}{{K}_{b} [ A ] + {K}_{a} [ B ] + [ A ] [ B ]} & ( 4 )\end{matrix}$$

In the above equations, [A] and [B] are the concentrations of ATP and peptide, respectively; Kₐ and K_{b} are the Km for ATP and peptide, respectively; and Kᵢₐ is the dissociation constant of A from the EA complex.

The initial reaction rates were obtained either as a function of product inhibition (ADP or phosphopeptide) or as a function of dead-end inhibition (AMP-PNP). In these studies one substrate is held constant while the other is varied against increasing concentrations of inhibitor. In the case of product inhibition, the non-varied substrate is held at saturating or non-saturating levels while in dead-end inhibition the non-varied substrate is held at saturating levels. The data were fit to a competitive inhibition model (equation 5), a noncompetitive inhibition model (equation 6), or an uncompetitive inhibition model (equation 7):

$$\begin{matrix}v = \frac{{V}_{max} [ S ]}{{K}_{m} ( 1 + \frac{[ I ]}{{K}_{is}} ) + [ S ]} & ( 5 ) \\ v = \frac{{V}_{max} [ S ]}{{K}_{m} ( 1 + \frac{[ I ]}{{K}_{is}} ) + [ S ] ( 1 + \frac{[ I ]}{{K}_{ii}} )} & ( 6 ) \\ v = \frac{{V}_{max} [ S ]}{{K}_{m} + [ S ] ( 1 + \frac{[ I ]}{{K}_{ii}} )} & ( 7 )\end{matrix}$$

where Kᵢᵢ and Kᵢₛ are the intercept and slope inhibition constants. The data were analyzed using Sigma Plot 2000 Enzyme Kinetics Module from SPSS Science (Richmond, Calif.).

Table II provides a summary of steady-state kinetic parameters for the peptides 1-4, ATP, and ATP in the absence of peptide.

TABLE II
Summary of Steady-State Kinetic Parameters

| varied | appKₘ | k_{cat} | K_{i peptide} | k_{cat}/Kₘ |
|---|---|---|---|---|
| substrate^{a} | (μM) | (sec⁻¹) | (μM) | (M⁻¹s⁻¹) |
| peptide1 | 6.5 ± 0.8 | 18 ± 1 | >2000 | 2 700 000 |
| peptide2 | 4.3 ± 0.8 | 16 ± 1 | 306 ± 57 | 3 600 000 |
| peptide3 | 420 ± 21 | 21 ± 1 | | 51 000 |
| peptide4 | 240 ± 16 | 14 ± 1 | | 58 000 |
| ATP^{b} | 49 ± 5 | 18 ± 1 | | 360 000 |
| ATP^{c} | 59 ± 8 | 0.16 ± 0.01 | | 2 600 |

^{a}Peptide1 and peptide2 fit to equation (2); peptide3, peptide4, and ATP fit to equation (1)
^{b}Peptide1 is present in this assay
^{c}no peptide present in assay

As shown in Table II, in the absence of peptide substrate, PKC-θ KD hydrolyzed ATP 110 times slower (0.16 sec⁻¹), than when peptide is present (18 sec⁻¹). The Kₘ for ATP of 59 μM (no peptide) and 49 μM (at saturating peptide 1) shows that there is no significant difference in the binding of ATP in the presence of peptide substrate. The steady state kinetic parameters for PKC-θ at saturating ATP are listed in Table II. Peptide 3 and peptide 4 show the highest Kₘ for PKC-θ with values of 420 μM and 240 μM, respectively. In contrast, Peptide 1 and peptide 2 have Kₘ values of 6.5 μM and 4.3 μM, respectively, and cause inhibition of the enzyme at high concentrations (Table II). The lower Kₘ values of the more basic peptides 1 and 2, implies a basic amino acid substrate peptide preference for PKC-θ.

Interestingly, the substrate inhibition observed with the longer more basic peptide 2 was more pronounced than for the shorter peptide 1 (Table II). Therefore, the kinetic parameters for PKC-θ (peptide 1 and ATP) were examined at increasing NaCl concentrations. The results of these studies are shown in Table III.

TABLE III
NaCl Effects on PCK-θ KD Steady-State kinetic parameters

| [NaCl] | appK_{m ATP}^{a,b} | k_{cat}^{b} | appK_{m peptide1}^{c} | K_{i peptide1}^{c} |
|---|---|---|---|---|
| mM | (μM) | (sec⁻¹) | (μM) | (μM) |
| 0 | 25 ± 5 | 7.5 ± 0.8 | 6.4 ± 4.2 | 129 ± 64 |
| 50 | 58 ± 8 | 18 ± 1 | 6.7 ± 2.8 | 201 ± 85 |
| 100 | 76 ± 7 | 22 ± 1 | 3.2 ± 1.0 | >2000 |
| 250 | 121 ± 16 | 24 ± 1 | 9.0 ± 1.2 | — |

^{a}at 0.2 mM peptide1
^{b}fit to equation (1)
^{c}fit to equation (2)

As shown in Table III, as the concentration of NaCl is increased, both the Kₘ for ATP and the turnover of the enzyme increases, while the Kₘ for peptide 1 remains relatively constant. Ionic strength effects on PKC-θ were also investigated by examining the NaCl effect on substrate inhibition that occurs when the substrate combines with the enzyme in a non-productive or dead-end complex. Substrate inhibition was observed for the preferred basic peptides 1 and 2, but not observed for the less optimal peptides 3 and 4 (see Table II). Furthermore, substrate inhibition was also found to be dependent on the ionic strength of the buffer. Substrate inhibition with peptide 1 diminishes as the NaCl concentration is increased to 250 mM (see Table III).

Thus, Table III shows that increases in buffer NaCl concentration increased the PKC-θ KD Kₘ for ATP and the enzyme turnover. An ionic strength effect was also observed on peptide 1 substrate inhibition. As the NaCl concentration increased, the substrate inhibition observed with peptide 1 diminished (see Table III). The nature of the salt (NaCl) and its effect on ion-pair formation can give insight to these observations. According to the Hofineister series of cations and anions, NaCl falls in the midpoint of kosmotrops and chaotrops (Cacace et al., Quarterly Reviews of Biophysics 30: 241-277, 1997). Therefore, NaCl should not salt out the enzyme nor denature the enzyme. However, increases in the ionic strength of the buffer would have an effect on the formation of ion-pairs (Park C. R. R., J. Am. Chem. Soc. 123: 11472-11479 (2001)). With the tyrosine kinase Csk, the addition of 50 mM NaCl had the effect of increasing the Kₘ for the substrate poly(Gly,Tyr), a negatively charged substrate; however, there was no effect on the Kₘ for ATP or the turnover of the enzyme (Cole et al., J. Biol. Chem. 269: 30880-30887, 1994). In the case of PKC-θ KD, the increase in Kₘ for ATP may be a result of two possibilities: 1) at 250 mM NaCl there is more productive binding of peptide 1 to the enzyme-ATP binary complex, and the Kₘ observed is a reflection of the actual Kₘ for ATP; or 2) the increase in ionic-strength effects ATP, a charged substrate, in the same manner as peptide 1. It is possible that the observed increase in Kₘ is a result of a combination of the above two possibilities. With the peptide 1 substrate, ion-pair formation (Columbic interactions) may be important in the binding of this substrate to the enzyme. At pH 7.5, a basic peptide such as peptide 1, would have a net positive charge. Therefore, increasing the NaCl concentration would result in a less favorable environment for ion-pair formation (Park C, R. R., J. Am. Chem. Soc. 123: 11472-11479 (2001)). If ion-pair formation contributes to the inhibition of peptide 1, then decreasing substrate inhibition with increasing NaCl is consistent with weakening of the Columbic interactions.

In determining the kinetic mechanism of PKC-θ, the initial velocity of the reaction with varied ATP was determined against fixed varied concentrations of peptide substrate at 100 mM NaCl. The assay was first done with peptide 1, however the resulting Lineweaver-Burk plot was difficult to interpret due to peptide 1 substrate inhibition (data not shown). Next, the intercept and slope replots of the Lineweaver-Burk plot (not shown) were performed against peptide 1.

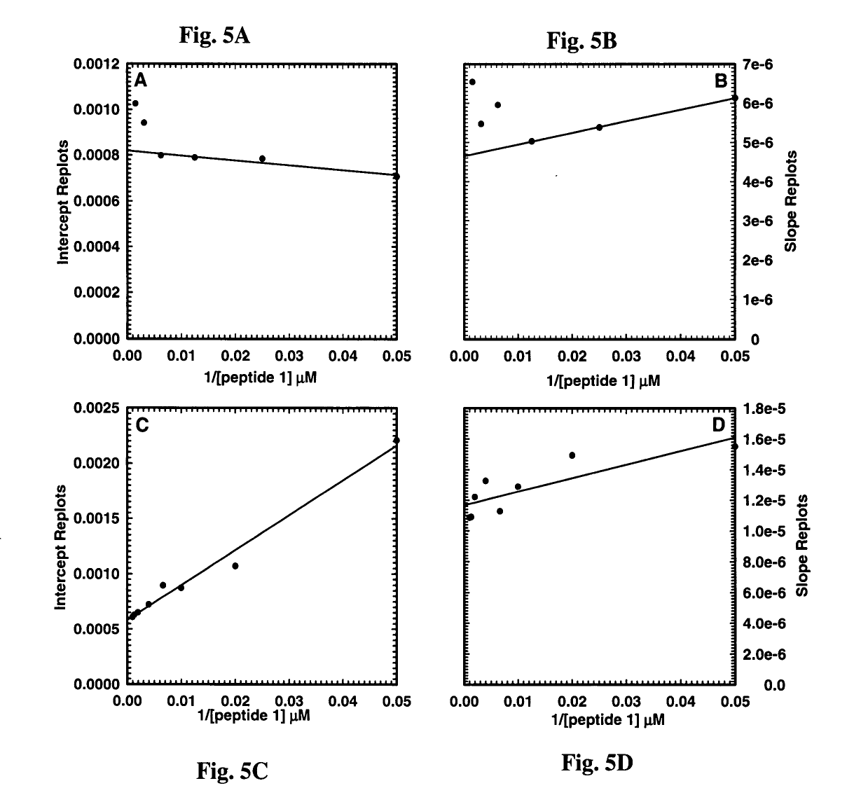

As shown in FIGS. 5A and 5B, the intercept and slope replots, respectively, against peptide 1 at 100 mM NaCl were non-linear. The initial velocity assays were also performed using peptide 3 under identical conditions. Varied ATP concentrations versus fixed varied peptide 3 concentrations resulted in an intersecting pattern on a Lineweaver-Burk plot (data not shown) that indicates a sequential kinetic mechanism. The Kᵢₐ value for ATP was 61±22 μM and the Kₐ for ATP was 118±17 μM. The initial velocity pattern with peptide 1 was then determined at 625 mM NaCl, as the increased salt concentration diminishes the substrate inhibition for peptide 1 (see Table III). The resulting Lineweaver-Burk plot produced an intersecting pattern as well (data not shown), consistent with a sequential kinetic mechanism when peptide 1 is the substrate. At high NaCl concentrations, intercept and slope replots of the Lineweaver-Burk plot (not shown) against peptide 1 were linear (see FIGS. 5C and 5D). The Kᵢₐ value for ATP of 66±32 μM obtained at high NaCl was found to be similar to the Kᵢₐ value for ATP of 61±22 μM with peptide 3 at 100 mM NaCl. This indicates that the increased ionic strength did not affect the dissociation constant of ATP from the enzyme-ATP complex. The Kₐ of ATP obtained at 625 mM NaCl was 321±19 μM, in contrast to the Kₐ of ATP at 100 mM NaCl of 118±17 μM. This is consistent with an increase in Kₘ for ATP as the ionic strength is increased (see Table III).

Dead-end inhibition studies identified ATP as the first substrate to bind PKC-θ KD. Accordingly, the substrate binding order in the sequential catalytic mechanism was next determined. AMP-PNP, a non-hydrolysable analogue of ATP, and peptide 5, with a serine to alanine change from peptide 1 (see Table I), were used for inhibition studies. The results of the inhibition studies are shown in Table IV.

TABLE IV
Inhibition Patterns and Constants^{a}

| | | sub- | pat- | Kᵢₛ | Kᵢᵢ | Equa- |
|---|---|---|---|---|---|---|
| Inhibitor | type | strate | tern^{b} | μM | μM | tion^{c} |
| ADP | product | ATP | c | 291 ± 24 | | 5 |
| ADP | product | pep- | — | | | |
| | | tide1 | | | | |
| ADP | product | pep- | nc | 494 ± 72 | 200 ± 29 | 6 |
| | | tide1^{d} | | | | |
| phospho- | product | ATP^{e} | uc | | 1600 ± 100 | 7 |
| peptide1 | | | | | | |
| phospho- | product | pep- | nc | 1700 ± 1100 | 1200 ± 800 | 6 |
| peptide1 | | tide3^{f} | | | | |
| phospho- | product | pep- | uc | | 2000 ± 400 | 7 |
| peptide1 | | tide3^{d,f} | | | | |
| AMP-PNP | dead- | ATP | c | 228 ± 29 | | 5 |
| | end | | | | | |
| AMP-PNP | dead- | pep- | — | | | |
| | end | tide1 | | | | |
| peptide5 | dead- | pep- | c | 10 ± 3 | | 5 |
| | end | tide1 | | | | |
| peptide5 | dead- | ATP | uc | | 1100 ± 100 | 7 |
| | end | | | | | |
| peptide5 | dead- | pep- | c | 4.4 ± 0.3 | | 5 |
| | end | tide3 | | | | |

^{a}NaCl concentration held at 100 mM
^{b}c, competitive; nc, noncompetitive; uc, uncompetitive; —, no inhibition observed
^{c}Data fit to equation number
^{d}ATP held at 0.1 mM
^{e}peptide3 held at 0.5 mM, peptide3 used due to low Kₘ of peptide1
^{f}peptide3 used due to substrate inhibition observed with peptide1

As shown in Table IV, AMP-PNP was found to be a competitive inhibitor of ATP with a Kᵢ value of 228 μM. There was no observed inhibition with AMP-PNP versus peptide, at saturating ATP. The peptide inhibitor, peptide 5, was shown to be a competitive inhibitor to peptide 1 as well as peptide 3 with Kᵢₛ values of 10 μM and 4.4 μM, respectively (Table IV). Peptide 5 was further shown to be an uncompetitive inhibitor against ATP with Kᵢᵢ values of 1100 μM (see Table IV). These results are consistent with an ordered sequential addition of substrates for PKC-θ where ATP associates first with enzyme followed by peptide.

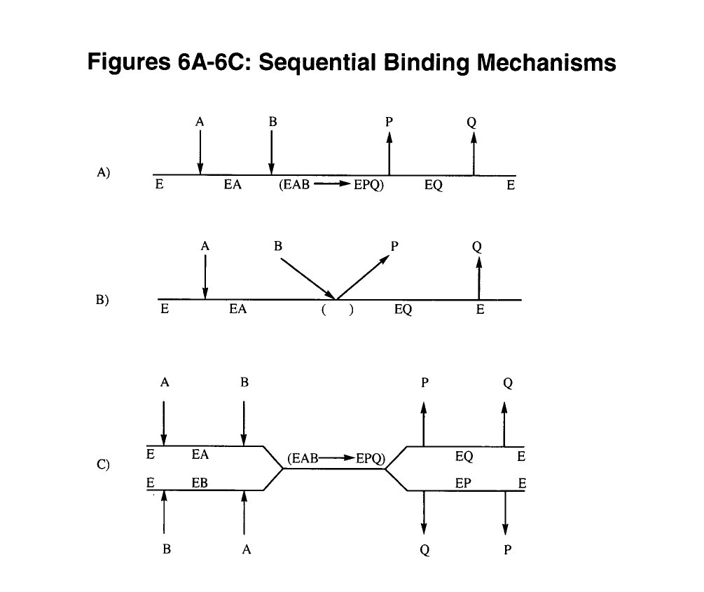

Because the PK/LDH coupled kinase assay consumes the catalytic product ADP, an HPLC assay was used to determine the inhibition patterns with ADP (see Table IV). ADP was found to be a competitive inhibitor against ATP at saturating peptide 1 with a Kᵢₛ of 291 μM. No inhibition was observed when ADP was assayed against peptide 1, at saturating ATP. When the assay was performed at non-saturating ATP (0.1 mM), a non-competitive pattern was observed with a Kᵢₛ of 494 μM and a Kᵢᵢ of 200 μM (see Table IV). These results with ADP rule out a random mechanism, as is depicted schematically in FIG. 6C, but are consistent with either a sequential ordered (depicted schematically as FIG. 6A) or Theorell-Chance (depicted schematically as FIG. 6B) kinetic mechanism, wherein ADP is the final product released. To further elucidate the kinetic mechanism, product inhibition assays with phosphopeptide 1 were performed. Due to the substrate inhibition observed with peptide 1, the product inhibition assays were done with peptide 3. Phosphopeptide 1 was a non-competitive inhibitor of peptide 3, with a Kᵢₛ of 1700 μM and a kᵢᵢ of 1200 μM, at a saturating ATP concentration (Table IV). At non-saturating ATP, an uncompetitive pattern was observed, with a Kᵢᵢ of 2000 μM. Phosphopeptide 1 was an uncompetitive inhibitor against ATP, at non-saturating peptide 3 (0.5 mM), with a kᵢᵢ of 1600 μM. The above results are more consistent with a sequential ordered Bi-Bi mechanism, with ADP as the last product released (see FIG. 6A).

The rate of the phospho-transfer step in the catalysis was investigated using the thio analog of ATP, ATPγS, and the results of these studies are shown in Table V.

TABLE V
Thio effect upon PKC-θ KD

| Kinetic | | | | |
|---|---|---|---|---|
| Parameter | [NaCl] mM | ATP^{a} | ATPγS^{a} | ratio ATP/ATPγS |
| appKₘ (μM) | 100 | 251 ± 24 | 120 ± 9 | 2.1 |
| appKₘ (μM) | 250 | 234 ± 19 | 130 ± 5 | 1.8 |
| rate^{b} | 100 | 179 ± 5 | 1.6 ± 0.1 | 112 |
| rate^{b} | 250 | 190 ± 4 | 1.3 ± 0.1 | 146 |

^{a}peptide1 used in assay
^{b}rate: peak area/(reaction time in minutes)([enzyme] nM)

As shown in Table V, substitution of ATPγS for ATP resulted in a large change in the k_{cat} of the reaction. The ATPγS reaction compared to the reaction with ATP is 112-fold and 146-fold slower in 100 mM NaCl and 250 mM NaCl, respectively. However, the Kₘ for ATP and ATPγS obtained using HPLC only differed by two-fold (Table V).

To determine if the chemical step is the lone contributor to the rate of the reaction, the effect of solvent viscosity on the steady state kinetic parameters for PKC-θ was determined. Two types of viscogens were employed in this study, the microviscogen sucrose and the macroviscogen Ficoll-400. Microviscogens directly affect the diffusion of small molecules while at the same time cause the viscosity effect observed with a viscometer (Blacklow et al., Biochemistry 27: 1158-1167 (1988)). Macroviscogens cause viscosity effects seen with a viscometer, but do not significantly affect the diffusion rate of the small molecules, thereby serving as a control for the microviscosity effect observed in the assay (Cole et al., J. Biol. Chem. 269: 30880-30887 (1994)). The steady state kinetic parameters of peptide 1, peptide 3, and ATP were determined in increasing solvent viscosity and at two different ionic strengths. The relative effect of solvent viscosity on the kinetic parameters, k_{cat} and k_{cat}/Kₘ, were plotted against relative viscosity of the buffer and fit to a linear regression.

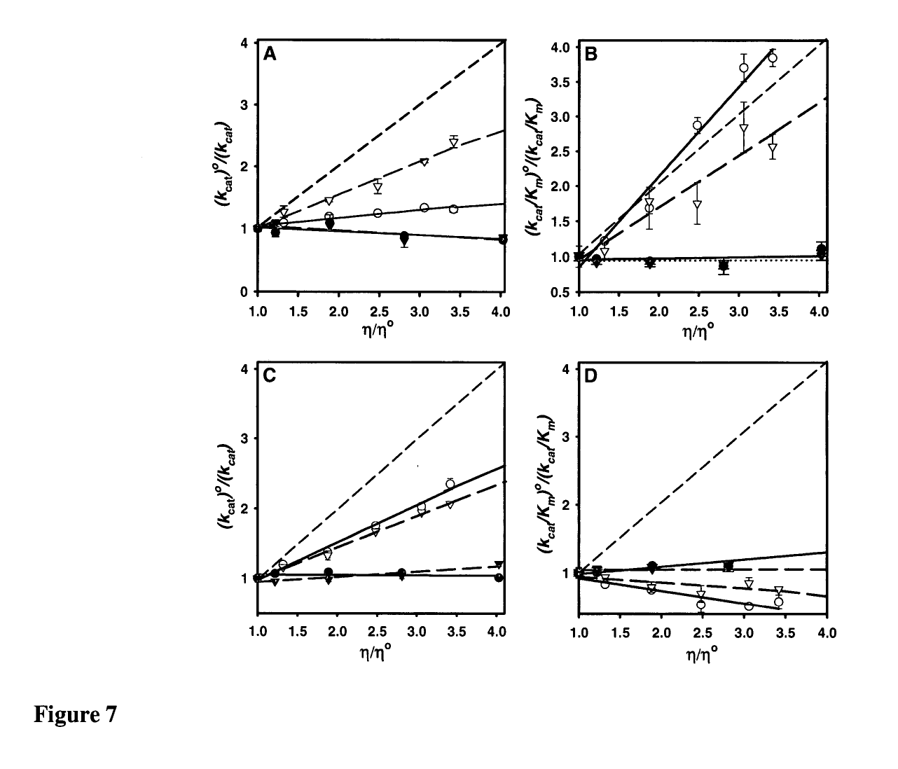

FIGS. 7A-7D show the solvent viscosity effects on k_{cat} and k_{cat}/Kₘ for PKC-θ KD. FIG. 7A shows the k_{cat} effect with varied peptide 1 with ATP held at 2.0 mM. FIG. 7B shows k_{cat}/Kₘ for ATP at 0.125 mM peptide 1. FIG. 7C shows the k_{cat} effect with varied peptide 3 with ATP held at 2.0 mM. FIG. 7D shows the k_{cat}/Kₘ for peptide 3 at 2.0 mM ATP. For FIGS. 7A-7D, the open circle symbol (∘) indicates 100 mM NaCl in increasing sucrose; the open inverted triangle symbol (∇) indicates 250 mM NaCl in increasing sucrose; the closed circle symbol (●) indicates 100 mM NaCl in increasing Ficoll 400; and the closed inverted triangle symbol (▾) indicates 250 mM NaCl in increasing Ficoll 400. The dashed line in FIGS. 7A-7D indicates a slope of 1. A slope of 1 indicates maximal effect of the microviscogen on the kinetic parameter. There was little effect on the enzymatic rate in the presence of the macroviscogen. As the microviscosity of the solvent increased there was a moderate effect seen on the (k_{cat})^{η} value. This was seen as a linear decrease in the observed rate of the enzyme with all three substrates studied at 100 mM NaCl and 250 mM NaCl. The slope [(k_{cat})^{η}] obtained under all the conditions varied from 0.38 to 0.54, implying that product release is partially rate-limiting (FIGS. 7A and 7C). A value of 0.8 to 1 indicates that product release is the catalytic rate-limiting step (Adams, J. A., Biochemistry 42: 601-607 (2003)).

The stickiness of a substrate can be determined by viscosity analysis. Briefly, for a sticky substrate the rate of product formation is faster than the rate of substrate dissociation, while a non-sticky substrate will dissociate from the enzyme faster than the rate of product formation (Cleland, W. W. (1986) Investigations of Rates and Mechanisms of Reactions, Vol. 6, Wiley-Interscience Publications, John Wiley & Sons, New York, N.Y.). The relative effect of increased solvent microviscosity on k_{cat}/Kₘ is plotted against relative solvent viscosity (FIGS. 7B and 7D). When the effect of microviscosity was plotted against relative solvent viscosity for peptide 1, the slope had a (k_{cat}/Kₘ)^{η} value of 0.86 in 250 mM NaCl (data not shown). Data were not obtained at 100 mM NaCl for peptide 1 due to the substrate inhibition observed at low ionic strength. Peptide 3, on the other hand, showed no solvent viscosity effect at either ionic strength (FIG. 7D). These studies imply that peptide 1 is a sticky substrate while peptide 3 is not sticky.

The kinetic mechanisms for several kinases have been reported (see, e.g., Wu et al., Biochemistry 41: 1129-1139 (2003); Trauger et al., Biochemistry 41: 8948-8953 (2003); Chen et al., Biochemistry 39: 2079-2087 (2000)). PKC-θ KD initial velocity plots, with both peptide substrates at high and low ionic strength resulted in graphs with lines intersecting left and below the ordinate. The pattern is a clear indication of a sequential mechanism that remained unaffected by the buffer ionic strength. In addition, there was no difference in the ATP Kᵢₛ values, 61 μM for peptide 3 at 100 mM NaCl and 66 μM for peptide 1 at 625 mM NaCl, further indicating that dissociation of ATP from the enzyme-ATP complex was not affected by the ionic strength. Under both conditions the Kᵢₛ value was found to be less than the Kₐ value of 118 μM and 321 μM, respectively, ruling out a rapid-equilibrium mechanism.

Dead-end inhibition and product inhibition studies (see Table IV) are consistent with a sequential ordered mechanism, wherein ATP is the first substrate to bind. The peptide inhibitor (peptide 5 in Table I) is competitive against both peptide substrates and un-competitive against ATP. While the ATP analog AMP-PNP was found to be competitive against ATP, there was no observed inhibition against peptide 1 up to 2.0 mM AMP-PNP at saturating ATP. A competitive pattern is observed when ATP is varied against ADP and there is no inhibition observed when peptide 1 is varied against ADP at saturating ATP. A non-competitive pattern is observed when peptide 1 is varied against ADP at non-saturating ATP. These inhibition studies rule out a random mechanism for PKC-θ KD and demonstrate that ADP is the last product released as shown in FIG. 6A.

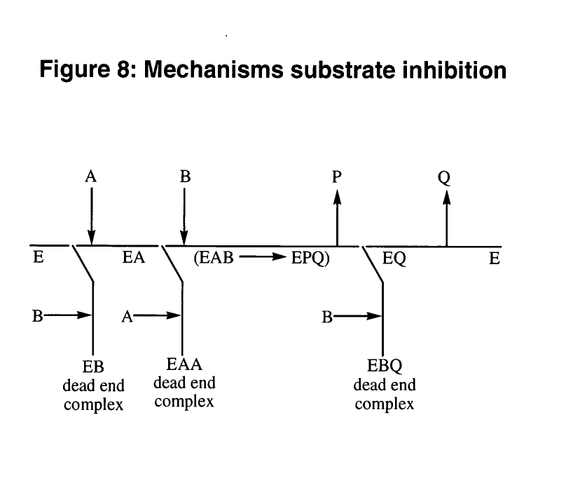

The initial velocity experiments at 100 mM NaCl with peptide 1 give some insight into the type of substrate inhibition observed. Briefly, there are three types of substrate inhibition observed in an ordered bireactant system (Segel, I. H. Enzyme Kinetics: Behavior and Analysis of Rapid Equilibrium and Steady-State Enzyme Systems, Whiely-Interscience, 1975) shown in FIG. 8. Two are substrate inhibition in which substrate B forms a dead-end EB complex or substrate A forms a EAA dead-end complex. The third is substrate inhibition in which B forms an EBQ dead-end complex. The formation of the EAA dead-end complex is ruled out because the first substrate to associate is ATP and no substrate inhibition is observed with ATP. FIGS. 5A-5D show the replots of the initial velocity data at 100 mM NaCl and 625 mM NaCl for peptide 1. As shown in FIGS. 5A and 5B, the effect of the inhibition is seen on both the slope and intercept replots at 100 mM NaCl (i.e. replots are not linear). However, at 625 mM NaCl (as shown in FIGS. 5C and 5D), the replots become linear indicating that the inhibition is abolished at high ionic strength up to 0.5 mM peptide 1. The effect of substrate inhibition on both the slope and intercept replots is consistent with non-competitive substrate inhibition (Cleland, W. W., Methods Enzymol. 63: 500-513 (1979)). Non-competitive substrate inhibition in an ordered sequential mechanism suggests the formation of the following types of non-productive enzyme complexes. Two are represented in FIG. 8, the EB and EBQ complex. A third possibility would be a non-productive EAB complex. This is a distinct possibility because at 0 mM NaCl the substrate inhibition is potent (0.129 mM) and the ATP concentration is ˜80×Kₘ (0.025 mM at 0 mM NaCl). In a sequential ordered mechanism in which ATP binds first there would be little free enzyme present to form the EB dead-end complex.

Phosphorothioates are used to study different types of enzymatic phosphotransfer reactions. The ATPγS reaction rate occurred 15 to 20 fold slower than the ATP reaction in the case of Csk kinase (Cole et al., J. Biol. Chem. 269: 30880-30887 (1994)). Similarly, in the enzymatic mechanism studies of phosphatidylinositol-specific phospholipase C (PLC), the reaction was slowed 10⁵ fold when the non-bridging oxygen was substituted with sulfur (Kravchuk et al., Biochemistry 40: 5433-5439 (2001)). Regardless of which ATP analog is used, ATP or ATPγS, the product, ADP, is identical. Thus, the catalytic rate will remain unaffected by product release. The thio effect observed for PKC-θ KD implies a contribution of the phospho-transfer chemistry to the overall rate of the enzymatic reaction. In addition, the large thio effect observed with PKC-θ argues against a Theorell-Chance ordered sequential kinetic mechanism depicted in FIG. 6B (McKay et al., Biochemistry 35: 8680-8685 (1996)). With a Theorell-Chance kinetic mechanism, the ternary complex is short-lived, therefore implying the chemical step is very fast.

The effect of solvent viscosity is a valuable tool in the determination of the rate-limiting step in an enzymatic reaction. The effect of increased solvent viscosity is seen on the non-chemical steps such as diffusion of the product from the enzyme and the diffusion of substrates to the enzyme active site (Blacklow et al., Biochemistry 27, 1158-1167 (1988)), and not on a unimolecular step such as the phosphotransfer step (Adams, J. A., Biochemistry 42: 601-607 (2003)). The PKC-θ solvent viscosity effect studies reported here indicate product release is a partly rate-limiting step in the catalysis.

The studies presented here illustrate PKC enzyme catalysis characteristics and kinetic mechanism, thereby providing insights into PKC isoforms and/or AGC family kinases that are highly similar to PKC. The results presented are consistent with a sequential ordered mechanism in which ATP binds first and ADP releases last. Phosphopeptide release and phosphotransfer contribute to the rate-limiting steps. Importantly, features potentially unique to PKC-θ are revealed in the phosphorylation studies of the kinase domain presented here. Taken together with the structural characteristics of PKC-θ (see Xu et al., J. Biol. Chem. 279(48): 50401-50409 (2004)), these findings have significant implications in advancing the selective targeting of this kinase for disease therapies.

### EXAMPLE 4

### Identification of PKC-θ Substrates

A peptide scanning array was next performed to identify peptide substrates for PKC-θ. To do this, as described in Example 3, the catalytic kinase domain of PKC-θ from residue 362 to residue 706 was cloned in the pET-16b expression vector. This vector introduced in frame a C-terminal hexa-Histidine tag to the expression clone. The plasmid was transformed in BL21-DE3 E. coli strain for over-expression. A 10-liter cell culture was initially grown at 37° C. up to an O.D. of 0.4 then the temperature was dropped to 25° C. before inducing the expression with 0.1 mM IPTG. The cells were grown for an additional 3 hours before harvest.

The cells were resuspended and lysed using a microfluidizer in Tris 25 mM pH 8.0, NaCl 25 mM, 2-mercaptoethanol 5 mM, imidazole 5 mM, ATP 50 μM and protease inhibitors. The lysate was applied for 1 hour at 4° C. by batch method to 20 ml (bed) of Nickel-NTA resin. The resin was subsequently poured as a chromatography column and washed extensively with the same buffer with imidazole increased to 25 mM. The step elution was realized with a 200 mM imidazole buffer. The protein was then immediately loaded onto an anion exchanger HQ and the column was washed with Tris 25 mM pH 8.0, NaCl 25 mM, DTT 5 mM, ATP 50 μM before being resolved by the application of a NaCl linear gradient up to 500 mM. The SDS-PAGE selected fractions were pooled and diluted two-fold with Tris 25 mM pH 8.0, DTT 5 mM and loaded onto a Heparin chromatography column. The protein fraction flowing through was immediately applied onto a hydroxyapatite column and washed extensively with Tris 25 mM pH 8.0, NaCl 50 mM, DTT 5 mM. A linear gradient of sodium phosphate from 0 to 100 mM eluted the target protein. The protein was then sized as a monomer on a superdex 200 size exclusion chromatography, dialyzed overnight against Tris 25 mM pH 8.0, NaCl 50 mM, DTT 5 mM and concentrated.

For peptide spot synthesis, Cellulose membranes modified with polyethylene glycol and Fmoc-protected amino acids were purchased from Intavis. Fmoc-protected-alanine was purchased from Chem-Impex (Wood Dale, Ill.). The arrays were defined on the membranes by coupling a β-alanine spacer and peptides were synthesized using standard DIC/HOBt (diisopropylcarbodiimide/hydroxybenzotriazole) coupling chemistry as described previously (see, e.g., Molina et al., Peptide Research 9: 151-155 (1996); and Frank, R., Tetrahedron 48: 9217-9232 (1992)). Activated amino acids were spotted using an Abimed ASP 222 robot. Washing and deprotection steps were done manually and the peptides were N-terminally acetylated after the final synthesis cycle.

Following peptide synthesis and side chain deprotection, kinase assays were performed. For these assays, the membranes were washed in methanol for 10 minutes and assay buffer (20 mM HEPES pH=7.5, 10 mM MgCl₂, 2 mM DTT, 100 mM NaCl and 20 μM ATP) for 10 minutes. The membranes were then incubated with 50 nM PKC-θ (kinase domain amino acid residues 362-706) C-terminal His-tagged expressed in E. coli and purified) in assay buffer containing 0.33 Ci/mMol γ-32P-ATP for 1 hour. The membranes were then washed 5 times with 200 mM sodium phosphate containing 0.1% Triton-X and 100 μM cold ATP and 3 times with ethanol. Next, the membranes were dried and imaged using a Biorad Fx.

Using these methods, 384 peptide sequences were tested. The phosphorylation of these peptides is shown in FIG. 9A. Of these 384 peptide sequences, the following were shown to be substrates for PKC-θ.

| | |
|---|---|
| FARKGSLRQKN | (SEQ ID NO: 6) |
| KKRESFKKSFK | (SEQ ID NO: 16) |
| QKRPSQRSKYL | (SEQ ID NO: 17) |
| KIQASFRGHMA | (SEQ ID NO: 18) |
| LSRTLSVAAKK | (SEQ ID NO: 19) |
| AKIQASFRGHM | (SEQ ID NO: 20) |
| VAKRESRGLKS | (SEQ ID NO: 21) |
| KAFRDTFRLLL | (SEQ ID NO: 22) |
| PKRPGSVHRTP | (SEQ ID NO: 23) |
| ATFKKTFKHLL | (SEQ ID NO: 24) |
| SPLRHSFQKQQ | (SEQ ID NO: 25) |
| KFRTPSFLKKS | (SEQ ID NO: 26) |
| IYRASYYRKGG | (SEQ ID NO: 27) |
| KTRRLSAFQQG | (SEQ ID NO: 28) |
| RGRSRSAPPNL | (SEQ ID NO: 29) |
| MYRRSYVFQT | (SEQ ID NO: 30) |
| QAWSKTTPRRI | (SEQ ID NO: 31) |
| RGFLRSASLGR | (SEQ ID NO: 32) |
| ETKKQSFKQTG | (SEQ ID NO: 33) |
| DIKRLTPRFTL | (SEQ ID NO: 34) |
| APKRGSILSKP | (SEQ ID NO: 35) |
| MYHNSSQKRH | (SEQ ID NO: 36) |
| MRRSKSPADSA | (SEQ ID NO: 37) |
| TRSKGTLRYMS | (SEQ ID NO: 38) |
| LMRRNSVTPLA | (SEQ ID NO: 39) |
| ITRKRSGEAAV | (SEQ ID NO: 40) |
| EEPVLTLVDEA | (SEQ ID NO: 41) |
| SQKRPSQRHGS | (SEQ ID NO: 42) |
| KPFKLSGLSFK | (SEQ ID NO: 43) |
| AFRRTSLAGGG | (SEQ ID NO: 44) |
| ALGKRTAKYRW | (SEQ ID NO: 45) |
| VVRTDSLKGRR | (SEQ ID NO: 46) |
| KRRQISIRGIV | (SEQ ID NO: 47) |
| WPWQVSLRTRF | (SEQ ID NO: 48) |
| GTFRSSIRRLS | (SEQ ID NO: 49) |
| RVVGGSLRGAQ | (SEQ ID NO: 50) |
| LRQLRSPRRTQ | (SEQ ID NO: 51) |
| KTRKISQSAQT | (SEQ ID NO: 52) |
| NKRRATLPHPG | (SEQ ID NO: 53) |
| SYTRESLARQV | (SEQ ID NO: 54) |
| NSRRPSRATWL | (SEQ ID NO: 55) |
| RLRRLTAREAA | (SEQ ID NO: 56) |
| NKRRGSVPILR | (SEQ ID NO: 57) |
| GKRRPSRLVAL | (SEQ ID NO: 58) |
| QKKRVSMILQS | (SEQ ID NO: 59) |
| RLRRLTAREAA | (SEQ ID NO: 60) |

Some of these peptides are shown in FIG. 9B, with the serine phosphorylated by PKC-θ indicated in bold-face type.

These peptide sequences phosphorylated by PKC-θ may be contained within the physiological substrate(s) of PKC-θ and as such may be a method to test physiological activity of inhibitors by testing inhibition of substrate phosphorylation in cells or in vivo. Furthermore, the physiological substrate(s) containing any of these amino acid residues may be a potential therapeutic target for inhibition or modulation in treatment of asthma by virtue of being a mechanism in the PKC-θ signaling pathway.

### EXAMPLE 5

### PKC-θ Activation Loop is Inducibly Phosphorylated and PKC-θ Membrane Translocation Occurs Upon IgE Receptor Crosslinking on BMMC

To look at the effect of the autophosphorylation of PKC-θ in the activation loop (i.e., on threonine 538) in asthma and allergic responses, the autophosphorylation of PKC-θ in the activation loop was determined following IgE receptor crosslinking in BMMC. For these studies, BMMC were isolated. To do this, bone marrow was extracted from the bones (femurs and tibias) of C57 Bl/6J mice (commercially available from The Jackson Laboratory, Bar Harbor, Me.), then plated at 5×10⁵ cells/ml in 10% HI FCS in DMEM+PS/gln and 50 μM βME+20 ng/ml recombinant murine IL-3 and 50 ng/ml recombinant murine SCF (commercially available from R&D Systems, Minneapolis, Minn.). Cells were passaged every 3-7 days. After 4 weeks, cultures were >95% mast cells (as determined by IgE receptor expression and c-kit expression). At this point, cells were cultured in the above media with murine IL-3 only at 50 ng/ml.

Isolated BMMC were treated with anti-DNP (Dinitrophenyl) IgE overnight in culture (approximately 16 hours). The following day, IgE receptor cross-linking was triggered with the addition of DNP-BSA to the cultures for 0, 2, 5, 30, and 90 minutes. Next, the treated BMMC were lysed in 1% NP-40 lysis buffer and cytosolic extracts (prepared as described in Example 1) were run on SDS-PAGE and transferred to nitrocellulose membranes. The nitrocellulose blots were probed first with anti-phosphoT₅₃₈ PKC-θ specific antibody (Cell Signaling Technology), then stripped and reprobed with anti-PKC-θ (commercially available from Santa Cruz).

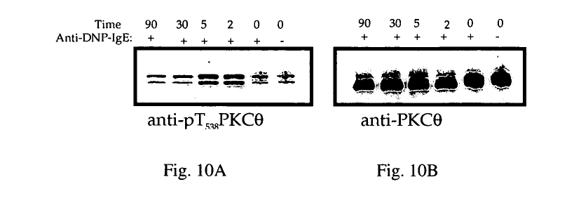

As shown in FIG. 10A, in the context of mast cell effector function in allergy and asthma, PKC-θ was found to be rapidly phosphorylated on threonine 538 in bone marrow derived mucosal mast cells upon IgE receptor cross-linking (FIG. 9A). Note that all BMMC expressed approximately equivalent amounts of PKC-θ, regardless of treatment regimen (see FIG. 10B). Unlike the sustained phosphorylation observed in T-cells (see FIGS. 1A-1C), phosphorylation at this site in mast cells was found to be rapid and transient (FIG. 10A). As shown in FIG. 10A, activation loop phosphorylation was found to occur as early as 2 minutes following IgE-receptor cross-linking, and returned to baseline levels after 30 minutes of cross-linking.

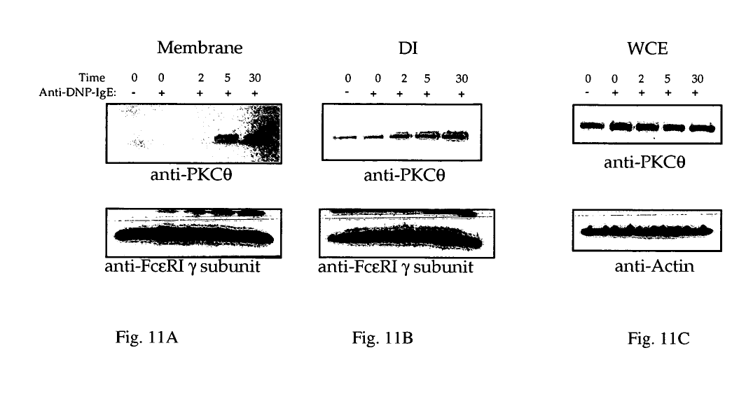

To determine whether PKC-θ membrane translocation occurred upon IgE receptor crosslinking in BMMC, BMMC (isolated as described above) were treated with anti-DNP IgE overnight in culture and stimulated with the addition of DNP-BSA for 0 minutes, 2 minutes, 5 minutes, or 30 minutes. Cells were then lysed and fractionated as described above in Example 1. The membrane fraction, the detergent-insoluble fraction (DI), and whole cell extracts (WCE) were resolved by SDS-PAGE, and then transferred to nitrocellulose. The nitrocellulose blots were then probed first with anti-PKC-θ (Santa Cruz), then stripped and reprobed with anti-FcεR1γ subunit for the membrane and DI fractions, and with anti-actin (Santa Cruz) for WCE to confirm equivalent amounts of expression of the IgE receptor (i.e., the FceRly subunit) on the membrane and DI fractions, and to confirm equivalent amounts of the cellular protein, actin, in the WCE. As shown in FIG. 11A, PKC-θ can be found in the membrane fraction of BMMC after 2 minutes of crosslinking the IgE receptor, and is clearly evident after 30 minutes crosslinking. Similarly, as shown in FIG. 11B, although PKC-θ can be observed at low levels in the DI fraction in unstimulated BMMC (i.e., 0 minutes in FIG. 11B), the amount of protein present increases following crosslinking of the IgE receptor. Note that equivalent amounts of the IgE receptor were present in all of the lanes of cells shown in FIGS. 11A and 11B.

Thus, similar to signaling in T cells, PKC-θ was found to rapidly translocate to the detergent insoluble fraction of the membrane upon IgE receptor crosslinking in mast cells. FIG. 11C confirms that this result was not simply due to a difference in the amount of PKC-θ in the different lanes, because all lanes of BMMC had equivalent amounts of PKC-θ in their whole cell extracts.

### EXAMPLE 6

### PKC-δ and PKC-β Distribution is Not Significantly Altered upon IgE Receptor Crosslinking on BMMCs

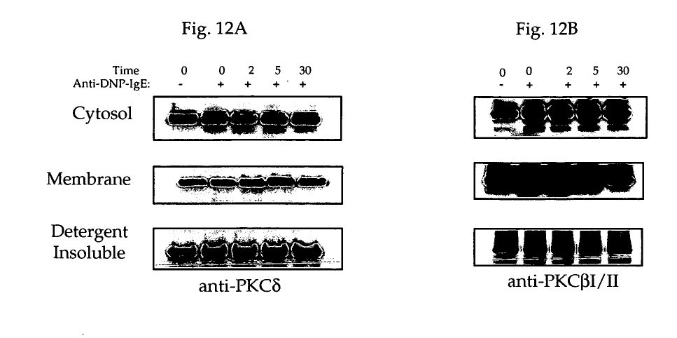

Two additional PKC family members, PKC-δ and PKC-β, have been implicated in mediating mast cell function following IgE receptor crosslinking (Nechushtan et al., Blood 95: 1752-1757 (2000); Kalesnikoff et al., J. Immunol. 168: 4737-4746 (2002). To determine whether other PKC family members were translocated to the membrane in BMMC following IgE receptor crosslinking, the fractions from the experiment described in Example 5 whose results are presented in FIGS. 11A-11C (i.e., the membrane, DI, and WCE fractions) were subjected to Western blotting analysis with the blots being probed for PKC-δ and PKC-β (instead of PKC-θ) using anti-PKC-δ (FIG. 12A) and anti-PKC-βI/βII (FIG. 12B) (both from Santa Cruz Biotechnology Inc.). As shown in FIGS. 12A and 12B, the inducible membrane translocation was not detected for PKC-β (FIG. 12A) and PKC-6 (FIG. 12B), as both are present in the cytosol, membrane, and detergent insoluble fractions in equivalent amounts before and after stimulation (i.e., crosslinking of the IgE receptor). These results demonstrate an important difference in the regulation of PKC-θ from both PKC-β and PKC-8 in IgE receptor signaling in mast cells.

### EXAMPLE 7

### Mast Cells from PKC-θ Knockout Mice are Different than Mast Cells from Wild-Type Mice

Studies of PKC-θ knockout mice have shown that PKC-θ is necessary for TCR-mediated T cell activation (Sun et al., Nature 404: 402-407 (2000)). A determination was made as to whether BMMC from PKC-θ knockout mice were different from BMMC from wild-type mice. To do this, PKC-θ knockout mice were obtained and T cell proliferation defects were confirmed according to the methods described in Sun et al., Nature 404: 402-407 (2000) (data not shown). Effects of PKC-θ deficiency were examined both in mucosal mast cells (MMC) and connective tissue mast cells (CTMC). These distinct mast cell phenotypes, differ in the composition and mediator content of the granules, and in their anatomical distribution (see Beil et al., Histol Histopathol. 15: 937-946 (2000)). MMC are found in lung and intestinal mucosa, and contain high levels of the protease tryptase. Their granules are rich in the proteoglycan chondroitin sulfate, allowing the cells to be stained with alcian blue. In contrast, CTMC, found in the gut, skin, and peritoneal cavity, express high levels of both tryptase and chymase, and release relatively higher levels of histamine than MMC. Their granules contain the proteoglycan heparan sulfate, allowing them to be stained with toluidine blue but not alcian blue. These two phenotypically distinct mast cell subsets are likely to differ in their in vivo function and regulation (see, e.g., Miller and Pemberton, Immunology 105: 375-90 (2002)), but the exact nature of these differences is still under investigation. In order to fully investigate the effects of PKC-θ on mast cells, each mast cell subset was examined in PKC-θ knock-out mice. MMC were derived in vitro from bone marrow progenitors. In contrast, CTMC could be recovered in mature form from the peritoneal cavity of mice.

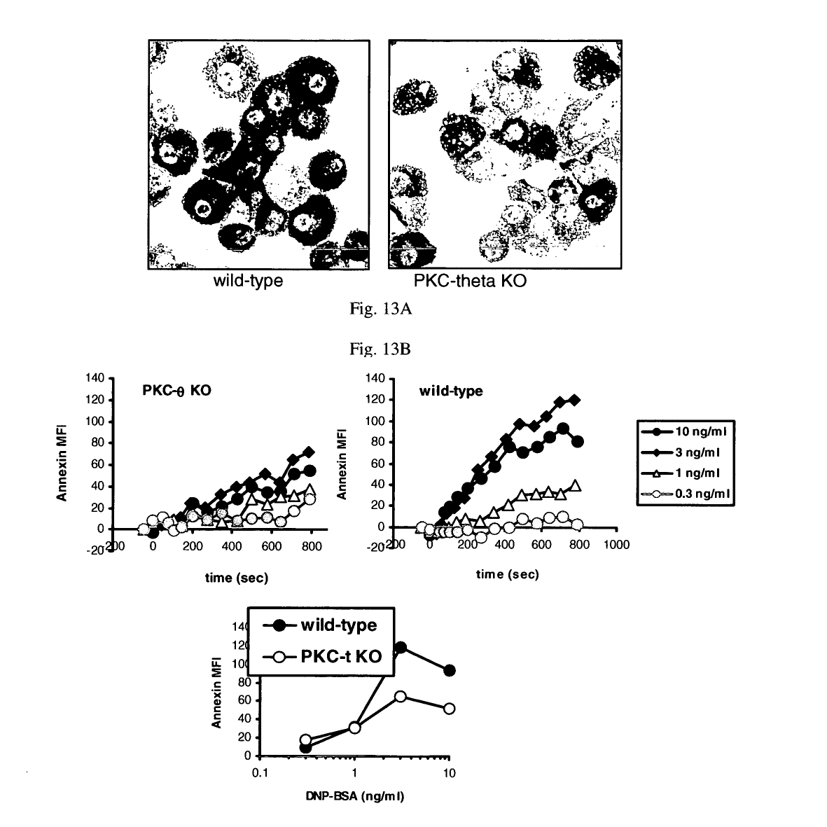

First, CTMC and BMMC from wild-type and PKC-θ knockout mice were compared phenotypically. To do this, CTMC were isolated by peritoneal lavage, spun onto microscope slides using a cytospin, and stained with toluidine blue, then counter-stained with safranin. Alternatively, the cells were stained with Wright's-Geimsa. Either staining protocol will identify the mast cell granules. No differences were apparent between wild-type and PKC-θ knock-out mice in number or percentage of peritoneal mast cells, or in granule density or distribution per cell (data not shown). BMMC of wild-type and PKC-θ knockout mice were isolated as described in Example 5 and spun onto microscope slides using a Cytospin, then stained with 1% alcian blue in 3% acetic acid for 5 minutes to stain the granules. Cells were counterstained with safranin. As shown in FIG. 13A, the BMMC from wild-type mice showed more granulation than BMMC from PKC-θ knockout mice.

Next, to quantitate the differences in granulation in BMMC following IgE receptor crosslinking, cell surface annexin staining was employed. Cell surface annexin staining increases with degranulation, in accordance with granule membrane fusion and phosphatidylserine exposure on the plasma membrane. For analysis of cell surface annexin expression, BMMC derived from wild-type or PKC-θ knockout mice were loaded with IgE anti-DNP by being treated overnight with 0.2 μg/ml IgE anti-DNP. The next day, cells were harvested and washed into PACM buffer. FITC-annexin was added and incubated with the cells for 3 minutes at 37° C. Time-based data acquisition was initiated on a FACScan equipped with 37° C. sample chamber, interrupted for addition of the indicated concentration of DNP-BSA to induce degranulation, and then resumed for 10 minutes per sample. Thus, the cells were induced to degranulate in the presence of FITC-labeled annexin. Expression of annexin at the cell surface is indicative of granule membrane fusion with the cell membrane upon degranulation. Mean fluorescence intensity was plotted as a function of time (FIG. 13B).

As shown in FIG. 13B, compared to wild-type, BMMC from PKC-θ knockout mice showed less cell surface annexin staining upon degranulation, which is consistent with their lower granule content by alcian blue staining (see FIG. 13A).

### EXAMPLE 8

### PKC-θ Knockout Mice have Reduced IgE Levels

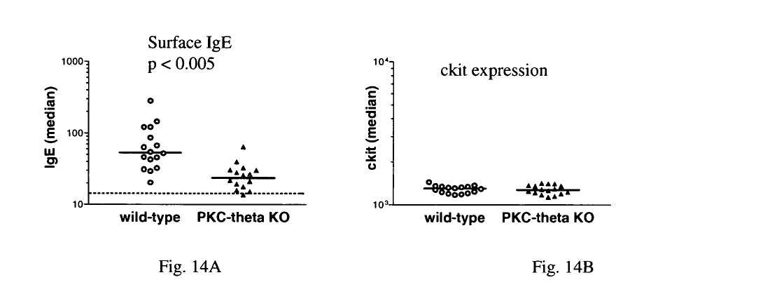

The levels of IgE receptor on CTMC from wild-type and PKC-θ knockout mice were next compared. To do this, peritoneal cavities of wild-type and PKC-θ knockout mice were lavaged with PIPES-EDTA buffer. Cells of the unfractionated peritoneal lavage from individual mice were washed into PBS containing 1% BSA (PBS-BSA) and incubated for 30 minutes on ice with 5 μg/ml IgE anti-DNP or no antibody. Cells were washed in PBS-BSA, then stained with FITC-labeled anti-mouse IgE and PE-labeled anti-ckit (BD-Pharmingen). Mean fluorescence intensity was quantitated by flow cytometry. As shown in FIG. 14A, compared to wild-type, PKC-θ knockout mice had significantly reduced levels of IgE bound to the CTMC surface. In contrast, there was no difference in level of expression of ckit (FIG. 14B). The level of IgE bound to surface IgE receptors of CTMC is related to circulating IgE levels in the animal. The reduced mast cell-bound IgE on CTMC suggests that PKC-θ knockout mice may have low levels of serum IgE.

Moreover, significant differences were observed between the serum antibody levels of wild-type mice and PKC-θ knockout mice. For these studies, the serum samples from wild-type and PKC-θ knockout mice was assayed for content of IgE, IgG1, and IgA. To do this, Maxi-Sorp ELISA plates (commercially available from Nunc, Rochester, N.Y.) were coated with anti-mouse IgE (commercially available from Pharmingen, San Diego, Calif.), anti-mouse kappa light chain (commercially available from Sigma, St. Louis, Mo.) for IgA, or anti-mouse IgG (Fab-specific; Sigma) for IgG1. Plates were washed with PBS containing 0.05% Tween-20 (PBS-Tween), then blocked with 0.5% gelatin in PBS for 2 hours at room temperature. Serum dilutions were added in PBS-Tween and incubated for 2 to 6 hours at room temperature. Binding was detected using specific biotinylated antibodies directed against mouse IgE or IgA (commercially available from Southern Biotechnology Associates, Inc., Birmingham, Ala.), or IgG1 (Pharmingen), followed by HRP-Streptavidin (Southern Biotechnology Associates) and Sure-Blue peroxidase substrate (commercially available from Kirkegaard and Perry Labs). Ig levels were quantitated using purified standards of the appropriate isotype (Pharmingen).

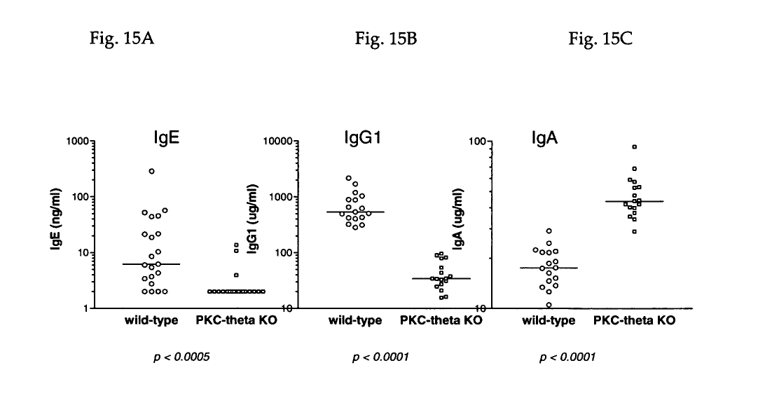

Thus, serum samples from individual mice were assayed for levels of the appropriate antibody isotype using specific ELISAs and quantitated by comparison to purified standards. As shown in FIG. 15A, IgE levels were significantly reduced in PKC-θ knockout mice. IgG1, which is often regulated coordinately with IgE, was also reduced (FIG. 15B). In contrast, IgA levels were higher in PKC-θ knockout mice as compared to wild-type (FIG. 15C).

Indeed, the CTMCs from PKC-θ knockout mice were found to have less degranulation in vitro with anti-IgE (data not shown), consistent with the decreased levels of circulating IgE and decreased mast cell-bound IgE (see FIGS. 14A and 15A).

Unlike the T cell activation defects reported in the PKC-θ knockout mice, no significant in vitro mast cell functional defects were observed in BMMC from PKC-θ knockout mice (data not shown). Because these cells are derived in vitro from progenitors in the bone marrow, they are not affected by in vivo levels of IgE, and can be loaded with exogenous IgE anti-DNP in vitro. Studies were next performed to determine if, upon IgE receptor cross-linking with DNP-BSA, the BMMC from PKC-θ knockout mice degranulated, generated leukotrienes, and produced cytokines at levels similar to those produced by BMMC from wild-type mice. For these studies, the following methods, mast cells were plated overnight in 10% HI FCS in DMEM+PS/gln and 50 μM βME+50 ng/ml recombinant murine IL-3+0.1 μg/ml anti-DNP-IgE (Sigma). Cells were washed in PACM (25 mM PIPES, pH 7.2 containing 110 mM NaCl, 5 mM KCl, 5 mM CaCl2, 2 mM MgCl2, and 0.05% BSA) and plated at a final concentration of 2.5×10⁵ cells/ml onto a titration of DNP-BSA (commercially available from Calbiochem, San Diego, Calif.) to crosslink the IgE receptor or Ionomycin.

For histamine, B-hexosiminidase and leukotriene production studies, cells were cultured for 30 min at 37° C. in the presence of DNP-BSA or Ionomycin, and then supernatants were harvested and either tested immediately or frozen. Results of degranuation and leukotriene production experiments showed that the BMMC of PKC-θ knockout mice had normal levels of degranulation and leukotriene production (data not shown). Maximum degranulation was determined by lysing an aliquot of cells with 0.1% Triton X-100. For beta-hexosaminidase, supernatants were incubated overnight at 37° C. with p-nitrophenyl N-acetyl B-D glucosaminide (Sigma) in 0.08 M sodium citrate pH 4.5. After 12-18 hours, reactions were stopped by addition of 1N NaOH, and beta-hexosaminidase was quantitated by reading absorption at 405 nm in a spectrophotometer. No significant differences were observed upon maximum degranulation (data not shown).

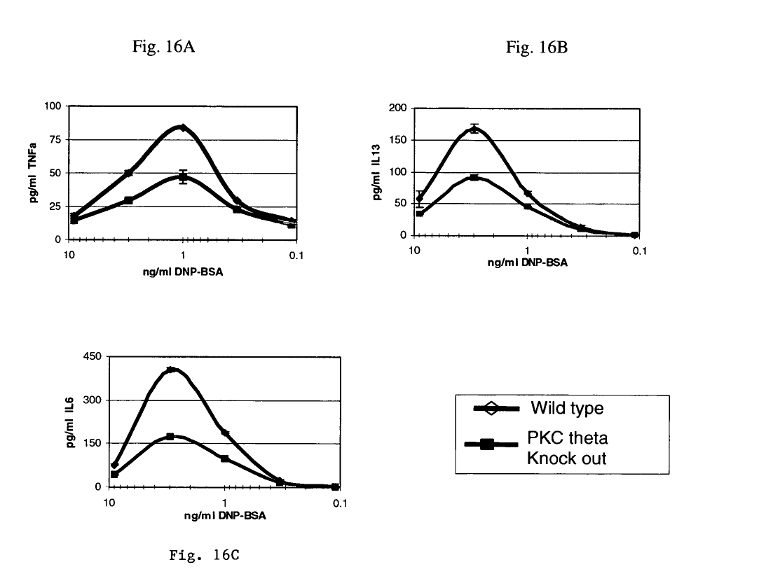

For cytokine production assays, cells cultured overnight in anti-DNP-IgE were incubated with DNP-BSA to trigger IgE receptor crosslinking for 6 hours before harvesting supernatants. Supernatants were assayed for leukotriene using an ELISA specific for LT(C4/D4/E4) (commercially available from ALPCO (Windham, N.H.), or for IL-6, IL-13 or GM-CSF using specific ELISA assays (R&D Systems, Minneapolis, Minn.). As shown in FIGS. 16A-16C, BMMC from PKC-θ knockout mice consistently produced lower levels of the cytokines TNF-α (FIG. 16A), IL-13 (FIG. 16B), and IL-6 (FIG. 16C) than BMMC from wild-type mice.

Next, spleens from C57BL/6 mice (The Jackson Laboratory, Bar Harbor, Me.) were made into a single cell suspension and CD4+ cells were isolated by anti-CD4 magnetic beads, followed by Detach-A-bead (Dynal Biotech) per manufacturer's instruction. The cells were either assayed as resting T cells, or activated to generate effector cells. Effector cells were generated by plating 6×10⁵ CD4+ cells/ml in DMEM medium supplemented with 10% FCS, 2 mM L-glutamine, 5×10⁻⁵M 2-mercapthoethanol, penicillin, streptomycin, sodium pyruvate and non-essential amino acids (all from Gibco Life Technologies, a subsidiary of Invitrogen, Carlsbad, Calif.) into 24-well plates that had been coated with 1 μg/ml anti-CD3 and 4 μg/ml anti-CD28 antibodies. Th1-skewed T cells were cultured in the presence of 30 ng/ml rmIL-12 (Wyeth, Cambridge, Mass.), 10 U/ml rhIL-2 (Invitrogen, Carlsbad, Calif.), and 5 μg/ml anti-mouse IL-4 antibodies. TH-2 skewed T cells were cultured in the presence of 40 ng/ml rmIL-4 (R&D Systems, Minneapolis, Minn.), 10 U/ml rhIL-2 (Invitrogen), and 5 μg/ml anti-mouse IFN-γ antibodies. After three days of stimulation, cells were expanded in the absence of IL-2 (5 U/ml) for an additional 3-4 days. Resting CD4+ T cells or Th1 or Th2 effector cells were plated at 1×10⁵ cells/well into 96-well flat-bottom plates that had been coated with 0.5 μg/ml of anti-CD3 (2C11). All antibodies were from B-D PharMingen, San Jose, Calif. After 3 days cell culture supernatant was harvested and assayed for IL-4 and IL-5 by cytokine bead assay (FACS).

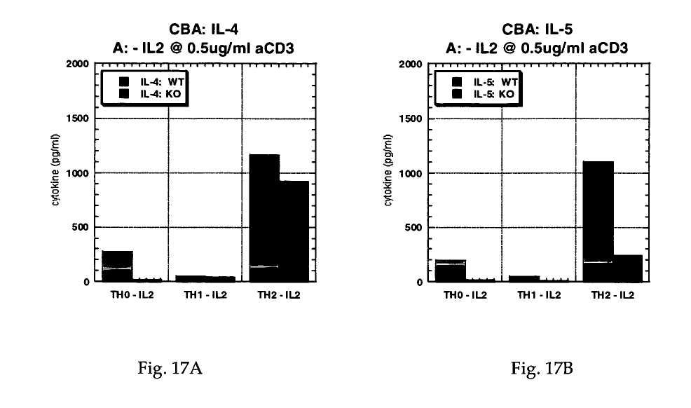

As shown in FIGS. 17A and 17B, T cell cytokine data from PKC-θ knockout mice showed that these mice produced reduced levels of both of these cytokines.

These results showing that that naïve PKC-θ knockout mice are deficient in circulating IgE and IgG1 levels are consistent with a role for PKC-θ in maintaining homeostatic levels of IL-4 in vivo. IL-4 is a Th2 cytokine which has a role in Ig (immunoglobulin) gene switching resulting in IgE and IgG1 synthesis (see Bacharier and Geha, J. Allergy Clin. Immunol. 105(2 Pt 2): S547-58 (2000); and Bergstedt-Lindqvist et al., Eur. J. Immunol. 18: 1073-1077 (1988)). The T cell expression of dominant negative gene constructs demonstrated that PKC-θ activates IL-4 gene transcription, in synergy with the GDP/GTP exhange factor Vav (see Hehner et al., J. Immunol. 164: 3829-3836 (2000)).

### EXAMPLE 9

### PKC-θ Knockout Mice do not have Increase in Ear Swelling in Response to anti-IgE or in the PCA Model in the Presence of Exogenous IgE

In order to determine whether PKC-θ is involved in IgE mediated mast cell activation, PKC-θ knockout mice were evaluated in a respiratory disease mouse model looking at passive cutaneous anaphylaxis (PCA). Thus, to determine whether PKC-θ is involved in IgE mediated mast cell activation, PKC-θ −/− (i.e., PKC-θ knockout) mice and C57BL/6 wild-type controls were challenged intradermally in the left ear with anti IgE (Pharmingen; 0.5 μg/kg in 20 μl of PBS). As a control, animals received 20 μl of PBS in the contralateral right ear. Prior to anti-IgE challenge, baseline ear thicknesses were determined using an engineer's micrometer, Upright Dial Gauge (commercially available from Mitutoyo (Japan)) measuring down to 0.0001″. Following challenge, ear thickness measurements were collected at 1 hour, 2 hours, 4 hours, and 6 hours and expressed as increase above baseline readings.

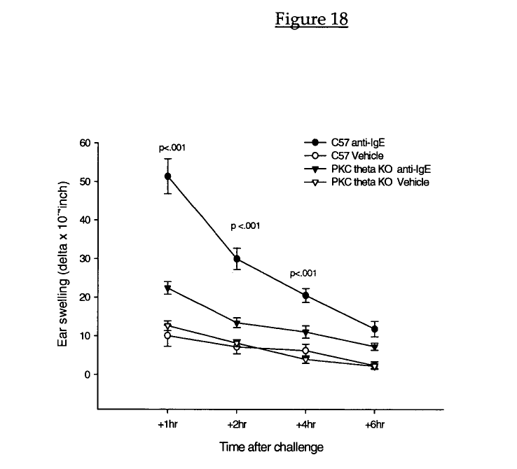

As shown in FIG. 18, PKC-θ knockout mice did not have increase in ear swelling in response to anti-IgE. Indeed, following anti-IgE challenge, ear swelling was approximately 2.5 fold greater in wild-type animals at the 1 hour time point compared to PKC-θ knockout animals (FIG. 18). The decreased ear swelling response in these mice is consistent with cell surface and circulating IgE levels. As described above, PKC-θ deficiency results in fewer mast cell granules (FIGS. 13A-13B) and lower IgE levels (FIG. 14A). These effects are likely in part due to the attenuated T cell dependent effects on modifying mast cell functions (Boyce, J. Allergy Clin. Immunol. 111: 24-32 (2003)).

To address whether PKC-θ is involved in IgE mediated mast cell signaling, mast cell-deficient Kit^{W}/Kit^{W-o} mice (commercially available from the Jackson Laboratory) were selectively repaired of their mast cell deficiency with either mast cells derived from PKC-θ knockout or wild-type mice. In other words, mast cells from PKC-0 knockout mice or normal, wild-type mice were transferred into the Kit^{W}/Kit^{W-o} mice (this adoptive transfer technique is reviewed in Galli and Lantz, Allergy. In Fundamental Immunology, W. E. Paul (ed.), pp. 1137-1184, Lippincott-Raven Press, Philadelphia Pa. 1999; and William and Galli, J. Allergy Clin. Immunol. 105(5): 847-859 (2000)). Briefly, 1×10⁶ BMMC from PKC-θ knockout or wild-type mice were resuspended in 20 μl of DMEM and injected into both the left and right ears (1×10⁶ BMCMC/ear) of 7 week old mast cell-deficient Kit^{W}/Kit^{W-o} mice (10 animals/group). After twelve weeks (an appropriate period of time to enable the adoptively transferred mast cells to mature within the connective tissue), mice were sensitized by intradermal injection into the left ear with IgE anti-DNP (5 μg/kg). As a control, animals received 0.9% saline into the right ear. Twenty-four hours later animals were challenged intravenously with DNP—HSA (10 mg/kg). Baseline ear measurements were collected prior to challenge and at 1 hour, 2 hours, 4 hours and 6 hours post challenge.

The results showed that Kit^{W}/Kit^{W-o} mice which were reconstituted with mast cells lacking PKC-θ showed no differences in terms of ear swelling compared to identically treated Kit^{W}/Kit^{W-o} mice that were reconstituted with wild-type mast cells (data not shown). These results suggest that the ear swelling deficiency in the PKC-θ mice is most likely due to T cell dependent effector function directly and/or indirectly on other immune cell types. Some T cell cytokines that are inhibited and impact immune cell function include IL-4, IL-5, TNF-a (FIGS. 17A, 17B, and data not shown).

However, the mast cell data suggests that inhibition of PKC-θ may directly modulate mast cell responses. As discussed above, PKC-θ was found to become rapidly phosphorylated on the activation loop upon IgE receptor crosslinking (see FIGS. 10A-10B). The subcellular distribution of PKC-θ, and not PKC-δ or PKC-βI/βII, is altered upon IgE receptor crosslinking (see FIGS. 11A-12B). Most importantly, there is attenuated cytokine production by PKC-θ knockout BMMC in response to IgE (FIGS. 16A-16C). These cells are derived in vitro from progenitors in the bone marrow and are not affected by in vivo levels of IgE.

In another experiment, PKC-θ knockout (i.e., PKC-θ −/−) mice and C57BL/6 wild-type controls were passively sensitized by intradermal injection into the left ear with monoclonal IgE anti-DNP (Sigma; 5 μg/kg in 20 μl of 0.9% saline) 24 hours prior to challenge. As a control animals received 20 μl of 0.9% saline into contralateral right ears. Twenty-four hours later, baseline ear measurements were collected, and then the animals were subjected to i.v. challenge with DNP—HSA (10 mg/kg in 100 μl of 0.9% saline). Over the following 6 hour period (i.e., readings at 1 hour, 2 hours, 4 hours, and 6 hour post-challenge) ear thickness measurements were collected as described above.

As shown in FIG. 19, PKC-θ knockout mice had significantly less ear swelling compared to identically treated wild-type counterparts. The results of these PCA studies (FIGS. 18 and 19) support the use of a PKC-θ small molecule antagonist in allergy and asthma in an animal disease model.

### EXAMPLE 10

### TH1 and TH2 T Cells from PKC-θ Knockout Mice Show Reduced Proliferation in Response to Stimuli as Compared to PKC-θ Wild-Type Mice

In order to determine whether differentiated T cells from PKC-θ knockout mice were normal in their ability to respond to stimuli, spleen cells from wild-type and PKC-θ knockout mice were differentiated in vitro to TH1 or TH2 populations to ascertain the proliferation defects of the T helper subsets of cells in the absence of PKC-θ expression. For these experiments, naïve T cells were isolated, and TH1 and TH2 effector cells were generated. To do this, spleens from C57/B6 mice (commercially available from Taconic, Germantown, N.Y.) were made into a single cell suspension. Red blood cells (RBC) were lysed with RBC lysing buffer (0.3 g/L ammonium chloride in 0.0M Tris-HCl buffer pH 7.5) and washed twice. CD4+ cells were isolated by anti-CD4 magnetic particles, followed by Detach-A-Bead (Dynal) per manufacturer's instruction (Dynal Biotech, Oslo, Norway). The cells were either assayed as naïve T cells, or activated to generate effector cells.

Effector cells were generated by activating naïve T cells by plating 6×10⁵ CD4+ isolates/ml in DMEM medium supplemented with 10% FCS, 2 mM L-glutamine, 5×10⁵ M 2-mercapthoethanol, penicillin, streptomycin, sodium pyruvate and non-essential amino acids (all from Gibco Life Technologies) into 24-well plates that had been coated with 1 μg/ml anti-CD3 and 4 μg/ml anti-CD28. TH1-skewed T cells (i.e., a population of T cells, the majority of which are TH1 cells) were generated by culturing the cells in the presence of 30 ng/ml recombinant murine IL-12 (Wyeth, Cambridge, Mass.), 10 U/ml recombinant human IL-2 (Invitrogen, Carlsbad, Calif.), and 5 μg/ml anti-mouse IL4 for 3 days. TH2-skewed T cells were generated by culturing the cells in the presence of 40 ng/ml recombinant murine IL-4 (R&D Systems, Minneapolis, Minn.), 10 U/ml recombinant human IL-2 (Invitrogen), and 5 μg/ml anti-mouse IFN-gamma for 3 days. All antibodies were from B-D PharMingen, San Jose, Calif.

For proliferation assays, TH1- or TH2-effector cells were plated at 1×10⁵ cells/0.2 ml/well into 96-well flat-bottom plates that had been coated with various concentrations of anti-CD3 (2C11) and in the presence or absence of soluble 5 μg/ml of anti-CD28 (clone 37.51) and/or 10 U/ml recombinant human IL-2 as indicated. On day 2, cultures were pulsed with 0.5 μCi of [³H]thymidine (Amersham Bioscience, Piscataway, N.J.) and harvested 6-8 hours later onto filters using a 96-well plate harvester. Incorporated radioactivity was measured using a liquid scintillation counter (Wallac, Gaithersburg, Md.). All antibodies were from B-D PharMingen, San Jose, Calif.

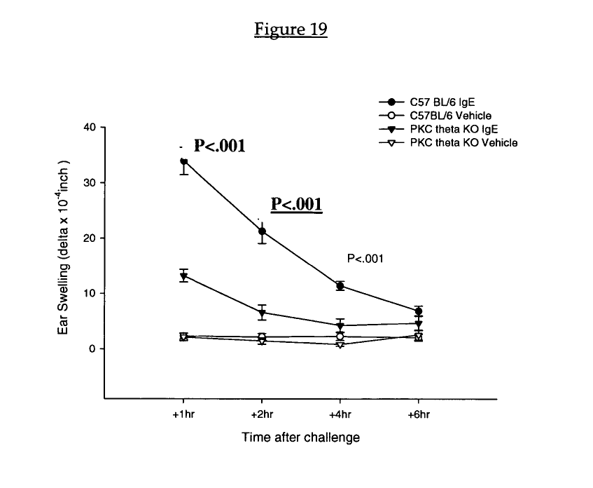

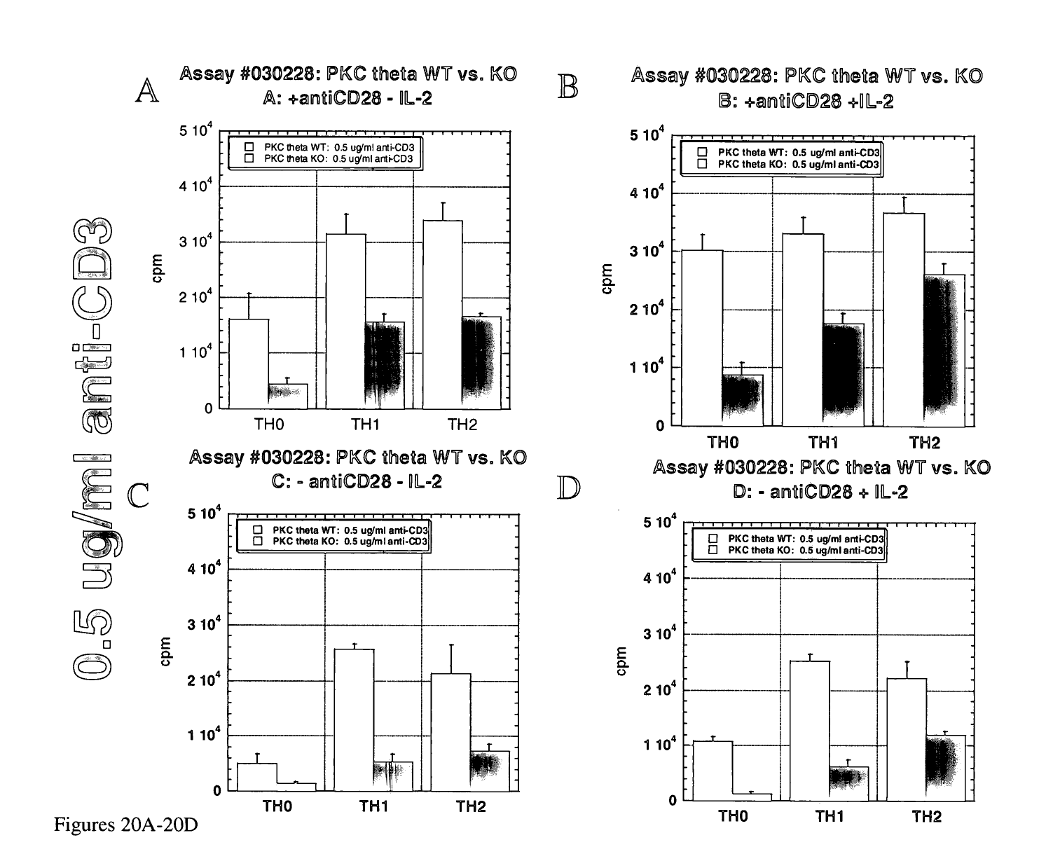

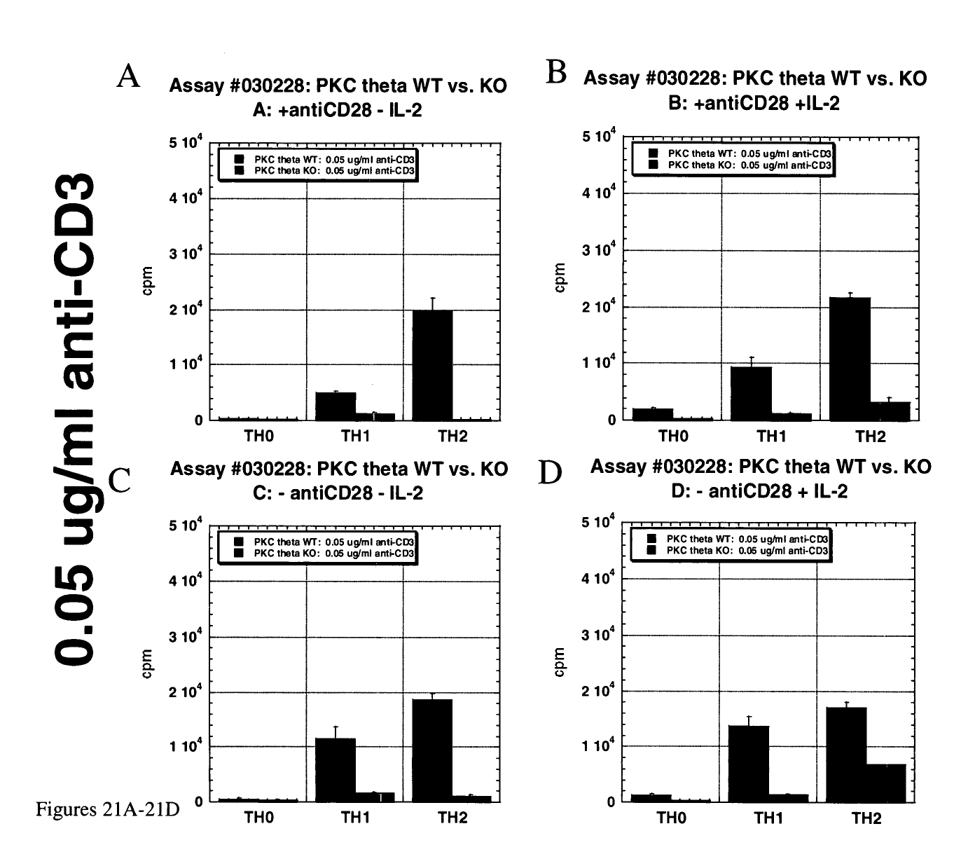

As shown in FIGS. 20 and 21, TH1 and TH2 cells from PKC-θ knockout mice were significantly reduced in the proliferation response to TCR stimulation at both optimal (0.5 μg/ml) and suboptimal (0.05 μg/ml) anti-CD3 signal strengths (FIG. 20C and FIG. 21C, respectively), as well as, upon TCR/CD28 co-stimulation (FIG. 20A and FIG. 21A, respectively). Addition of exogenous IL-2 to support T cell proliferation by a PKC-θ independent pathway partially overcame the reduced proliferation responses of naïve TH0, TH1, and TH2 cells from PKC-θ knockout mice, but only in conjunction with TCR/CD28 co-stimulations at the optimal 0.5 μg/ml anti-CD3 signal (FIG. 20B with anti-CD28 and FIG. 20D without anti-CD28). At suboptimal 0.05 μg/ml anti-CD3, CD28 co-stimulation failed to overcome the almost complete lack of proliferation of cells from PKC-θ knockout mice (FIG. 21A). In these conditions there is a very modest effect of exogenous IL-2 (FIGS. 21B and 21D), with proliferation of TH2 cells from PKC-θ knockout mice remaining approximately 30% of the proliferation of TH2 cells from wild-type mice.

These results, together with the inhibition of IL-2 production by PKC-θ knockout T cells (see, e.g., Sun et al., Nature 404: 402-407 (2000)), suggest that TCR stimulation-induced proliferation cannot be sustained by TH0, TH1, and TH2 cells if PKC-θ activity is inhibited in these cells. In conjunction with reduced TH2 cytokine production, these T helper cells, therefore, will not function as optimal effector cells mediating T cell dependent pathways in asthma and allergic disease physiology. From these findings, a further aspect of the invention is to target PKC-θ in TH2 T cells. Thus, the invention further provides a therapeutic intervention for preventing and/or alleviating the symptoms of asthma by targeting PKC-θ in TH2 T cells.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

1. A method for identifying a modulator of a PKC-θ protein, comprising:
(a) contacting a PKC-θ protein, or a functional fragment thereof, with a test agent; and
(b) determining if the test agent modulates the kinase activity of the PKC-θ protein, or the functional fragment thereof,
wherein a change in the kinase activity of the PKC-θ protein, or the functional fragment thereof, in the presence of the test agent is indicative of a modulator of a PKC-θ protein.

2-19. (canceled)

20. The method of claim 1, wherein step (a) further comprises contacting PKC-θ protein, or the functional fragment thereof, with a test agent and a PKC-θ substrate.

21. The method of claim 20, wherein the kinase activity is the phosphorylation of the PKC-θ substrate.

22. The method of claim 20, wherein the PKC-θ substrate comprises an R-X-X-S motif or an R-X-X-T motif, wherein R is arginine, X is either an unknown or any known amino acid, S is serine, and T is threonine.

23. The method of claim 22, wherein the PKC-θ substrate has an amino acid sequence selected from the group consisting of KKRFSFKKSFK (SEQ ID NO: 5), FARKGSLRQKN (SEQ ID NO: 6), FARKGSLRQ (SEQ ID NO: 15), KKRFSFKKSFK (SEQ ID NO: 16), QKRPSQRSKYL (SEQ ID NO: 17), KIQASFRGHMA (SEQ ID NO: 18), LSRTLSVAAKK (SEQ ID NO: 19), AKIQASFRGHM (SEQ ID NO: 20), VAKRESRGLKS (SEQ ID NO: 21), KAFRDTFRLLL (SEQ ID NO: 22), PKRPGSVHRTP (SEQ ID NO: 23), ATFKKTFKHLL (SEQ ID NO: 24), SPLRHSFQKQQ (SEQ ID NO: 25), KFRTPSFLKKS (SEQ ID NO: 26), IYRASYYRKGG (SEQ ID NO: 27), KTRRLSAFQQG (SEQ ID NO: 28), RGRSRSAPPNL (SEQ ID NO: 29), MYRRSYVFQT (SEQ ID NO: 30), QAWSKTTPRR1 (SEQ ID NO: 31), RGFLRSASLGR (SEQ ID NO: 32), ETKKQSFKQTG (SEQ ID NO: 33), DIKRLTPRFTL (SEQ ID NO: 34), APKRGSILSKP (SEQ ID NO: 35), MYHNSSQKRH (SEQ ID NO: 36), MRRSKSPADSA (SEQ ID NO: 37), TRSKGTLRYMS (SEQ ID NO: 38), LMRRNSVTPLA (SEQ ID NO: 39), ITRKRSGEAAV (SEQ ID NO: 40), EEPVLTLVDEA (SEQ ID NO: 41), SQKRPSQRHGS (SEQ ID NO: 42), KPFKLSGLSFK (SEQ ID NO: 43), AFRRTSLAGGG (SEQ ID NO: 44), ALGKRTAKYRW (SEQ ID NO: 45), VVRTDSLKGRR (SEQ ID NO: 46), KRRQISIRGIV (SEQ ID NO: 47), WPWQVSLRTRF (SEQ ID NO: 48), GTFRSSIRRLS (SEQ ID NO: 49), RVVGGSLRGAQ (SEQ ID NO: 50), LRQLRSPRRTQ (SEQ ID NO: 51), KTRKISQSAQT (SEQ ID NO: 52), NKRRATLPHPG (SEQ ID NO: 53), SYTRFSLARQV (SEQ ID NO: 54), NSRRPSRATWL (SEQ ID NO: 55), RLRRLTAREAA (SEQ ID NO: 56), NKRRGSVPILR (SEQ ID NO: 57), GKRRPSRLVAL (SEQ ID NO: 58), QKKRVSMILQS (SEQ ID NO: 59), and RLRRLTAREAA (SEQ ID NO: 60).

24-27. (canceled)

28. A method for identifying a modulator of a PKC-θ protein, comprising:
(a) contacting a cell expressing a PKC-θ protein, or a functional fragment thereof, with a test agent; and
(b) determining if the test agent reduces the autophosphorylation of the PKC-θ protein, or the functional fragment thereof, in the cell,
wherein a change in the autophosphorylation of the PKC-θ protein, or the functional fragment thereof, in the presence of the test agent is indicative of a modulator of a PKC-θ protein.

29. The method of claim 28, wherein the modulator of a PKC-θ protein that reduces the kinase activity is an inhibitor of the PKC-θ protein, or the functional fragment thereof.

30. The method of claim 28, wherein the modulator of a PKC-θ protein that increases the kinase activity is an activator of the PKC-θ protein, or the functional fragment thereof.

31. The method of claim 28, wherein the PKC-θ protein is a full-length PKC-θ protein.

32. The method of claim 28, wherein the PKC-θ protein is a functional variant of a full-length PKC-θ protein.

33. The method of claim 28, wherein the functional fragment is a PKC-O kinase domain.

34. The method of claim 28, wherein the determining step comprises comparing the kinase activity of the test agent relative to the absence of the test agent.

35. The method of claim 28, wherein the modulator of a PKC-θ protein is useful for treating asthma.

36. The method of claim 35, further comprising assessing the efficacy of the test agent identified in step (b) in an in vitro or in vivo asthma model, wherein a test agent that shows an increased efficacy in the in vitro or in vivo asthma model as compared to a control agent is identified as being useful for treating asthma.

37. The method of claim 28, wherein the cell is a prokaryotic cell.

38. The method of claim 37, wherein the prokaryotic cell is E. coli.

39. The method of claim 28, wherein the autophosphorylation of the PKC-θ protein, or the functional fragment thereof, occurs on an amino acid residue of SEQ ID NO:1 selected from the group consisting of the serine residue at position 695, the serine residue at position 685, the threonine residue at position 538, and the threonine residue at position 536.

40. The method of claim 39, wherein the autophosphorylation occurs on the threonine residues at position 538 of SEQ ID NO:1.

41. A method for treating asthma, comprising administering to a mammal suffering from asthma or suffering from an asthma symptom a therapeutically effective amount of an agent that reduces the kinase activity of a PKC-θ protein, or a functional fragment thereof, or reduces the production of a functional PKC-θ protein.

42-60. (canceled)

60. An isolated mast cell lacking expression of endogenous PKC-θ protein.

61. (canceled)


SEQUENCE LISTING (72 sequences)

SEQ ID NO: 1 - protein, 706 aa, Homo sapiens
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 2 - protein, 707 aa, Mus sp.
MSPFLRIGLSNFDCGTCQACQGEAVNPYCAVLVKEYVESENGQMYIQKKPTMYPPWDSTFDAHINKGRVMQIIVKGKNVDLISETTVELYSLAERCRKNNGRTEIWLELKPQGRMLMNARYFLEMSDTKDMSEFENEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARSLRDSEHIFREGPVEIGLPCSTKNETRPPCVPTPGKREPQGISWDSPLDGSNKSAGPPEPEVSMRRTSLQLKLKIDDFILHKMLGKGSFGKVFLAEFKRTNQFFAIKALKKDVVLMDDDVECTMVEKRVLSLAWEHPFLTHMFCTFQTKENLFFVMEYLNGGDLMYHIQSCHKFDLSRATFYAAEVILGLQFLHSKGIVYRDLKLDNILLDRDGHIKIADFGMCKENMLGDAKTNTFCGTPDYIAPEILLGQKYNHSVDWWSFGVLVYEMLIGQSPFHGQDEEELFHSIRMDNPFYPRWLEREAKDLLVKLFVREPEKRLGVRGDIRQHPLFREINWEELERKEIDPPFRPKVKSPYDCSNFDKEFLSEKPRLSFADRALINSMDQNMFSNFSFINPGMETLICS

SEQ ID NO: 3 - DNA, 2705 bp, Homo sapiens
tgctcgctccagggcgcaaccatgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctgaatcttgcccctccagagacaggaaagaatttgccttctccctgggaactggttcaagagacactgcttgggttcctttttcaacttggaaaaagaaagaaacactcaacaataaagactgagacccgttcgcccccatgtgactttatctgtagcagaaaccaagtctacttcactaatgacgatgccgtgtgtctcgtctcctgacatgtctcacagacgctcctgaagttaggtcattactaaccatagttatttacttgaaagatgggtctccgcacttggaaaggtttcaagacttgatactgcaataaattatggctcttcacctgggcgccaactgctgatcaacgaaatgcttgttgaatcaggggcaaacggagtacagacgtctcaagactgaaacggccccattgcctggtctagtagcggatctcactcagccgcagacaagtaatcactaacccgttttattctatcctatctgtggatgtataaatgctgggggccagccctggataggtttttatgggaattctttacaataaacatagcttgtacttg

SEQ ID NO: 4 - DNA, 3313 bp, Mus sp.
cttgggtcgccaggcccgcgccagtccccgccatccgagcaacagcggcgctgctctgggaccgcggccgcgacaccagggaacaaccatgtcaccgtttcttcgaatcggtttatccaactttgactgtgggacctgccaagcttgtcagggagaggcagtgaacccctactgcgctgtgcttgtcaaagagtatgtggaatcagaaaatgggcagatgtacatccagaaaaagccaaccatgtacccaccttgggacagcacctttgacgcccacattaacaagggaagggtgatgcagatcatcgtgaagggcaagaatgtagacctcatctcagaaacaaccgtggaactctactccctggcggagagatgccgcaagaacaatgggcggacagaaatatggttagagctgaaacctcaaggccgaatgctaatgaatgcaagatactttctggaaatgagtgacacaaaggacatgagtgagtttgagaatgaaggattctttgcactgcatcagcgccgaggagccatcaaacaggccaaagtccaccatgtcaagtgtcacgagttcacggccacctttttccctcaacccacattttgctctgtctgccatgaatttgtctggggcctgaacaagcagggttaccagtgccgacagtgtaatgcagcgattcacaagaagtgcattgataaagtgatagccaagtgcacaggatccgcaatcaatagccgagaaaccatgttccataaggagagattcaagatcgacatgccacacagattcaaagtctacaactacaagagtccaaccttctgtgagcactgtggtaccctgctctgggggctggcgaggcaaggactcaaatgtgatgcatgtggcatgaacgtccaccaccgatgccagacaaaggttgccaatctttgtggtataaaccagaagctaatggctgaagcactagcgatgattgaaagcacccaacaggctcgctccttacgagattcagaacacatcttccgagaaggcccagttgaaattggtctcccatgctccaccaaaaacgaaaccaggccaccatgcgtaccaacacctgggaaaagagaaccccagggcatttcctgggattcccctttggatgggtcaaataaatcggccggtcctcctgaacccgaagtgagcatgcgcaggacttcactgcagctgaaactgaagatcgatgacttcatcctgcacaagatgttgggaaaaggaagttttggcaaggtcttcctggcagagttcaagagaaccaatcagtttttcgcaataaaagccttaaagaaagatgtggtgttgatggatgatgacgtcgagtgtacaatggtggaaaagagggttctgtccttggcatgggagcatccatttctaacacacatgttctgcacattccagaccaaggaaaatctctttttcgtgatggagtatctcaatggaggcgacttaatgtaccacatccaaagttgccacaaatttgatctttccagagccacgttttatgctgctgaggtcatccttggtctgcagttccttcattccaaaggaattgtctacagggacctgaagcttgataatatcctgttagacagagatggacatatcaaaatagcagactttgggatgtgcaaagagaacatgctaggagatgcgaagacaaatactttctgtggaactcctgactacattgctccggagatcttgctgggtcagaagtacaaccattccgtcgactggtggtccttcggggtgctcgtttatgagatgctgattggccagtcccccttccacgggcaggacgaggaggagctgttccactccatccgcatggacaaccccttctacccgaggtggctcgaaagggaggccaaggaccttctagtgaagctttttgtgagagaacctgagaagaggctgggagtgagaggagacatccgccagcatcctttgtttcgagagatcaactgggaagagcttgaaaggaaagagattgacccacccttcagaccaaaagtgaaatcaccatatgactgtagcaatttcgacaaggaattcctaagtgagaaaccccggctatcattcgccgacagagcactcatcaacagcatggaccagaacatgttcagcaacttttccttcattaacccagggatggagactctcatttgctcctgaacctcatctgtctccagactggaagggatttgccttctctctgggaactggttcaagtaacacttctgggggtgggggtctctttttcacgttagagaagaaaagaaacactgcaaaggcagggaggactcctgagctccttgtgtgacttgttacctacagcacaaaccacgcctacttcactaatgacatcatccctaatgacatcatcccgttatatctcctggaatctctcacagcagcccttgaagttagatcattattaactctagtcatttacttgaaagatggttcccgatgctgtgaaagattcgaaatgcagttctgctcttgccctagacaacagctgctggttggtgatgaaccaaggcgcaagtggaacagatttctcaagactggagcagtgatcgcctgttatagaagtcaattccactcaaccacagagaaggaaccactaagccacgttgatgtgtgcatgtctgtggaaatgtcgatgacagaagggagggaaaggggaagctctgagcagattgtaatgggaagctctccaataaacatagcatgaaacttgaaatttacaaatctgttcattctggctagccccaaaattcccaaggcagaggaaagtaaagggcagtgagcttagcagagccctttgtcgccaacagggaagggtaaggatgtcgcctacgtggaacaacttatacacacagaaggaaagtataaccaacaagggcagggtggtttacagctgccaatcaaacctgccctcccccctctgttctcagttgatctctctgtcagcgtaggtaggcactcattaccatcctcccatcatacaagaaataaaatgcatgactcttctaagataaagaaaaccaatcccttatcacgttgttcccagtgatttgatggcaaataagtccctccttaggcatcctgcaagacaacccaacccatgcatgctatttgcagtagtcagtcctgttgagttagagtcctaactatacacaatatcgtgcgatgtttatatatgttgatgagatgttgtgatgataacgtggatatgtaaaagggaataaaagaagaaagaaagatgcc

SEQ ID NO: 5 - protein, 11 aa, Artificial Sequence
  Description of Artificial Sequence Synthetic peptide
KKRFSFKKSFK

SEQ ID NO: 6 - protein, 11 aa, Artificial Sequence
  Description of Artificial Sequence Synthetic peptide
FARKGSLRQKN

SEQ ID NO: 7 - DNA, 37 bp, Artificial Sequence
  Description of Artificial Sequence Synthetic oligonucleotide
cagaatatgttcaggaacttttccttcatgaaccccg

SEQ ID NO: 8 - protein, 12 aa, Artificial Sequence
  Description of Artificial Sequence Synthetic peptide
QNMFRNFSFMNP

SEQ ID NO: 9 - DNA, 36 bp, Artificial Sequence
  Description of Artificial Sequence Synthetic oligonucleotide
ggagatgccaagacgaataccttctgtgggacacct

SEQ ID NO: 10 - protein, 12 aa, Artificial Sequence
  Description of Artificial Sequence Synthetic peptide
GDAKTNTFCGTP

SEQ ID NO: 11 - DNA, 2148 bp, Artificial Sequence
  Description of Artificial Sequence Synthetic nucleotide sequence
atgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttgagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctacccatacgatgttccagattacgcttag

SEQ ID NO: 12 - protein, 715 aa, Artificial Sequence
  Description of Artificial Sequence Synthetic amino acid sequence
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISYPYDVPDYA

SEQ ID NO: 13 - DNA, 2148 bp, Artificial Sequence
  Description of Artificial Sequence Synthetic nucleotide sequence
atgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttgagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaatatgggccttaaagaaagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctacccatacgatgttccagattacgcttag

SEQ ID NO: 14 - protein, 715 aa, Artificial Sequence
  Description of Artificial Sequence Synthetic amino acid sequence
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIWALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISYPYDVPDYA

SEQ ID NO: 15 - protein, 9 aa, Artificial Sequence
  Description of Artificial Sequence Synthetic peptide
FARKGSLRQ

SEQ ID NO: 16 - protein, 11 aa, Artificial Sequence
  Description of Artificial Sequence Synthetic peptide
KKRFSFKKSFK

SEQ ID NO: 17 - protein, 11 aa, Artificial Sequence
  Description of Artificial Sequence Synthetic peptide
QKRPSQRSKYL

SEQ ID NO: 18 - protein, 11 aa, Artificial Sequence
  Description of Artificial Sequence Synthetic peptide
KIQASFRGHMA

SEQ ID NO: 19 - protein, 11 aa, Artificial Sequence
  Description of Artificial Sequence Synthetic peptide
LSRTLSVAAKK

SEQ ID NO: 20 - protein, 11 aa, Artificial Sequence
  Description of Artificial Sequence Synthetic peptide
AKIQASFRGHM

SEQ ID NO: 21 - protein, 11 aa, Artificial Sequence
  Description of Artificial Sequence Synthetic peptide
VAKRESRGLKS

SEQ ID NO: 22 - protein, 11 aa, Artificial Sequence
  Description of Artificial Sequence Synthetic peptide
KAFRDTFRLLL

SEQ ID NO: 23 - protein, 11 aa, Artificial Sequence
  Description of Artificial Sequence Synthetic peptide
PKRPGSVHRTP

SEQ ID NO: 24 - protein, 11 aa, Artificial Sequence
  Description of Artificial Sequence Synthetic peptide
ATFKKTFKHLL

SEQ ID NO: 25 - protein, 11 aa, Artificial Sequence
  Description of Artificial Sequence Synthetic peptide
SPLRHSFQKQQ

SEQ ID NO: 26 - protein, 11 aa, Artificial Sequence
  Description of Artificial Sequence Synthetic peptide
KFRTPSFLKKS

SEQ ID NO: 27 - protein, 11 aa, Artificial Sequence
  Description of Artificial Sequence Synthetic peptide
IYRASYYRKGG

SEQ ID NO: 28 - protein, 11 aa, Artificial Sequence
  Description of Artificial Sequence Synthetic peptide
KTRRLSAFQQG

SEQ ID NO: 29 - protein, 11 aa, Artificial Sequence
  Description of Artificial Sequence Synthetic peptide
RGRSRSAPPNL

SEQ ID NO: 30 - protein, 10 aa, Artificial Sequence
  Description of Artificial Sequence Synthetic peptide
MYRRSYVFQT

SEQ ID NO: 31 - protein, 11 aa, Artificial Sequence
  Description of Artificial Sequence Synthetic peptide
QAWSKTTPRRI

SEQ ID NO: 32 - protein, 11 aa, Artificial Sequence
  Description of Artificial Sequence Synthetic peptide
RGFLRSASLGR

SEQ ID NO: 33 - protein, 11 aa, Artificial Sequence
  Description of Artificial Sequence Synthetic peptide
ETKKQSFKQTG

SEQ ID NO: 34 - protein, 11 aa, Artificial Sequence
  Description of Artificial Sequence Synthetic peptide
DIKRLTPRFTL

SEQ ID NO: 35 - protein, 11 aa, Artificial Sequence
  Description of Artificial Sequence Synthetic peptide
APKRGSILSKP

SEQ ID NO: 36 - protein, 10 aa, Artificial Sequence
  Description of Artificial Sequence Synthetic peptide
MYHNSSQKRH

SEQ ID NO: 37 - protein, 11 aa, Artificial Sequence
  Description of Artificial Sequence Synthetic peptide
MRRSKSPADSA

SEQ ID NO: 38 - protein, 11 aa, Artificial Sequence
  Description of Artificial Sequence Synthetic peptide
TRSKGTLRYMS

SEQ ID NO: 39 - protein, 11 aa, Artificial Sequence
  Description of Artificial Sequence Synthetic peptide
LMRRNSVTPLA

SEQ ID NO: 40 - protein, 11 aa, Artificial Sequence
  Description of Artificial Sequence Synthetic peptide
ITRKRSGEAAV

SEQ ID NO: 41 - protein, 11 aa, Artificial Sequence
  Description of Artificial Sequence Synthetic peptide
EEPVLTLVDEA

SEQ ID NO: 42 - protein, 11 aa, Artificial Sequence
  Description of Artificial Sequence Synthetic peptide
SQKRPSQRHGS

SEQ ID NO: 43 - protein, 11 aa, Artificial Sequence
  Description of Artificial Sequence Synthetic peptide
KPFKLSGLSFK

SEQ ID NO: 44 - protein, 11 aa, Artificial Sequence
  Description of Artificial Sequence Synthetic peptide
AFRRTSLAGGG

SEQ ID NO: 45 - protein, 11 aa, Artificial Sequence
  Description of Artificial Sequence Synthetic peptide
ALGKRTAKYRW

SEQ ID NO: 46 - protein, 11 aa, Artificial Sequence
  Description of Artificial Sequence Synthetic peptide
VVRTDSLKGRR

SEQ ID NO: 47 - protein, 11 aa, Artificial Sequence
  Description of Artificial Sequence Synthetic peptide
KRRQISIRGIV

SEQ ID NO: 48 - protein, 11 aa, Artificial Sequence
  Description of Artificial Sequence Synthetic peptide
WPWQVSLRTRF

SEQ ID NO: 49 - protein, 11 aa, Artificial Sequence
  Description of Artificial Sequence Synthetic peptide
GTFRSSIRRLS

SEQ ID NO: 50 - protein, 11 aa, Artificial Sequence
  Description of Artificial Sequence Synthetic peptide
RVVGGSLRGAQ

SEQ ID NO: 51 - protein, 11 aa, Artificial Sequence
  Description of Artificial Sequence Synthetic peptide
LRQLRSPRRTQ

SEQ ID NO: 52 - protein, 11 aa, Artificial Sequence
  Description of Artificial Sequence Synthetic peptide
KTRKISQSAQT

SEQ ID NO: 53 - protein, 11 aa, Artificial Sequence
  Description of Artificial Sequence Synthetic peptide
NKRRATLPHPG

SEQ ID NO: 54 - protein, 11 aa, Artificial Sequence
  Description of Artificial Sequence Synthetic peptide
SYTRFSLARQV

SEQ ID NO: 55 - protein, 11 aa, Artificial Sequence
  Description of Artificial Sequence Synthetic peptide
NSRRPSRATWL

SEQ ID NO: 56 - protein, 11 aa, Artificial Sequence
  Description of Artificial Sequence Synthetic peptide
RLRRLTAREAA

SEQ ID NO: 57 - protein, 11 aa, Artificial Sequence
  Description of Artificial Sequence Synthetic peptide
NKRRGSVPILR

SEQ ID NO: 58 - protein, 11 aa, Artificial Sequence
  Description of Artificial Sequence Synthetic peptide
GKRRPSRLVAL

SEQ ID NO: 59 - protein, 11 aa, Artificial Sequence
  Description of Artificial Sequence Synthetic peptide
QKKRVSMILQS

SEQ ID NO: 60 - protein, 11 aa, Artificial Sequence
  Description of Artificial Sequence Synthetic peptide
RLRRLTAREAA

SEQ ID NO: 61 - protein, 345 aa, Artificial Sequence
  Description of Artificial Sequence Synthetic PKC-O sequence
PELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 62 - protein, 347 aa, Artificial Sequence
  Description of Artificial Sequence Synthetic PKC-O sequence
MGPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 63 - protein, 353 aa, Artificial Sequence
  Description of Artificial Sequence Synthetic PKC-O sequence
MGPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLISHHHHHH

SEQ ID NO: 64 - protein, 11 aa, Artificial Sequence
  Description of Artificial Sequence Synthetic peptide
NFSFMNPGMER

SEQ ID NO: 65 - protein, 12 aa, Artificial Sequence
  Description of Artificial Sequence Synthetic peptide
ALINSMDQNMFR

SEQ ID NO: 66 - protein, 20 aa, Artificial Sequence
  Description of Artificial Sequence Synthetic peptide
TNTFCGTPDYIAPEILLGQK

SEQ ID NO: 67 - protein, 11 aa, Artificial Sequence
  Description of Artificial Sequence Synthetic peptide
YLRRASVAQLT

SEQ ID NO: 68 - protein, 11 aa, Artificial Sequence
  Description of Artificial Sequence Synthetic peptide
PTSPGSLRKHK

SEQ ID NO: 69 - protein, 13 aa, Artificial Sequence
  Description of Artificial Sequence Synthetic peptide
RFARKGSLRQKNV

SEQ ID NO: 70 - protein, 8 aa, Artificial Sequence
  Description of Artificial Sequence Synthetic peptide
LKRSLSEM

SEQ ID NO: 71 - protein, 16 aa, Artificial Sequence
  Description of Artificial Sequence Synthetic peptide
RTPKLARQASIELPSM

SEQ ID NO: 72 - protein, 9 aa, Artificial Sequence
  Description of Artificial Sequence Synthetic peptide
FARKGALRQ